(19) United States
(12) Patent Application Publication
(10) Pub. No.: US 2011/0268818 A1
(43) Pub. Date: Nov. 3, 2011

(54) NGAL-BINDING SIDEROPHORES AND USE THEREOF TO TREAT IRON DEFICIENCY AND IRON OVERLOAD

(76) Inventors: Jonathan Barasch, New York, NY (US); Shixian Deng, White Plains, NY (US); Guanhu Bao, New York, NY (US); Donald W. Landry, New York, NY (US)

(21) Appl. No.: 13/119,682

(22) PCT Filed: Sep. 18, 2009
(86) PCT No.: PCT/US09/57543

§ 371 Date: Jul. 7, 2011

Related U.S. Application Data
(60) Provisional application No. 61/097,909, filed on Sep. 18, 2008.
(60) Provisional application No. 61/180,674, filed on May 22, 2009.

(51) Int. Cl. A61K 33/26; A61P 3/02; C07K 14/47; A61P 3/00; A61K 38/17; G01N 33/68

(52) U.S. Cl. 424/646; 514/5.4; 436/501; 530/410

(57) ABSTRACT

The invention provides compositions comprising a lipocalin, such as NGAL, and a mammalian siderophore that are useful as iron chelators and iron donors. The invention also provides mammalian siderophore compounds of Formula (I): The invention further provides, methods of treatment and methods of diagnosis.

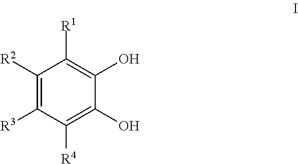

This invention was made with government support under grants DK-55388 and DK-58872 awarded by the NIH. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

The transport of iron inside and among cells poses a significant problem because free ferric iron is insoluble (<10⁻¹⁸M) in aerobic solutions at physiologic pH. Solubilization of iron is also problematic because, when bound to some chelators, iron remains capable of catalyzing reactions that produce toxic oxygen radicals. Consequently, specialized mechanisms are required to sequester iron in order to control its chemical reactivity, while enhancing its solubility for transport. These specialized mechanisms are found in proteins such as transferrin and ferritin, which utilize conserved motifs to bind iron. Other proteins, rather than directly binding iron, utilize common cofactors to chelate iron such as sulfides or heme groups which are embedded within the protein.

In mammals, transport of iron among cells has been considered to be largely mediated by transferrin, but recent studies in transferrin receptor 1 deleted mice, where organogenesis still continues, indicated that both transferrin and non-transferrin mechanisms must coexist to solubilize and transport iron in a non-reactive form. Hypo-transferrinemic mice (hpx) and humans demonstrate normal organogenesis and knockouts of the transferrin receptor TfR1 survive to mid-gestation. Recent studies have shown that TfR1^{-/-} embryonic stem cells populate most organs of the embryo. These findings require non-transferrin-based iron transport pathways as alternative mechanisms of iron acquisition, though few candidates have been proposed.

The mechanisms of iron transport in damaged cells are also largely unknown. Many authors discuss the presence of catalytically active iron in damaged cells and it is well known that after organ damage, catalytic iron is found in the blood and in the urine and accumulates abnormally in cells, but the molecular nature of this iron and its disposition are currently speculative.

Because free ferric iron is nearly insoluble at physiologic pH and must be chelated in order to be transported, there is a need for iron chelators and donors which can transport iron with improved solubility, bioavailability and safety.

### SUMMARY

The present invention is based, in part, on the discovery of a family of catechol-related iron-binding compounds that bind with high affinity to lipocalin proteins, such as neutrophil gelatinase-associated lipocalin (“NGAL”), and the discovery that complexes comprising these catecholate compounds and a lipocalin are able to bind to, transport, and release iron in vivo. Thus, the catechol-related compounds of the invention, and complexes containing such catechol-related compounds and a lipocalin, may be used as iron chelators and/or iron donors and may be useful in the treatment of various conditions, diseases and disorders associated with excessive iron levels and/or iron deficiency.

In one aspect, the invention provides a composition comprising, consisting of, or consisting essentially of a compound of Formula I, I(a), I(b), II, or III. In a preferred embodiment, the present invention provides a composition comprising, consisting of, or consisting essentially of a compound of Formula I, I(a), I(b), II, or III and a lipocalin. In a further preferred embodiment, the invention provides a composition comprising, consisting of, or consisting essentially of a compound of Formula I, I(a), I(b), II, or III, and a lipocalin and iron. In preferred embodiments, the iron in such compositions is bound to the compound of Formula I, I(a), I(b), II, or III. The chemical structures of Formulae I, I(a), I(b), II, and III are provided in the Detailed Description section of this application.

In preferred embodiments, the catecholate compounds of the invention are selected from the group consisting of: catechol, 3-methylcatechol, 4-methylcatechol, rosmarinic acid, myricetin, epigallocatechin gallate, pyrogallol, 2,3-dihydroxybenzoic acid and ellagic acid.

In further preferred embodiments, the lipocalin is NGAL, or a homolog, variant, derivative, fragment, or mutant thereof that has the ability to bind to the catecholate compounds of the invention.

In one aspect, the invention provides a method for treating iron deficiency in a subject, the method comprising administering to a subject in need thereof a composition provided by the invention, wherein the composition comprises iron.

In one embodiment of the invention, the iron deficiency to be treated is associated with anemia, cancer, HIV/AIDS, hepatitis, autoimmune diseases, cardiovascular disease, bleeding, a dietary deficiency, an effect of a drug, a malabsorption syndrome, fever, or any combination thereof.

In another aspect, the invention provides a method for treating iron overload in a subject, the method comprising administering to a subject in need thereof a composition provided by the invention.

In one embodiment of the invention, the iron overload to be treated is associated with sickle cell disease, thalassemia, hemochromatosis, aceruloplasminemia, atransferrinemia, blood transfusion, diet, hemodialysis, chronic liver disease, porphyria cutanea tarda, post-portacaval shunting, dysmetabolic iron overload syndrome, or any combination thereof.

In another embodiment of the invention, the iron overload to be treated is associated with a condition or disease that affects the kidney selected from the group consisting of acute or chronic kidney disease, contrast induced nephropathy, acute glomerulonephritis, acute tubular nephropathy (ATN) and diabetes mellitus.

In another embodiment, the present invention provides crystals comprising a compound of the invention, the lipocalin NGAL, and iron. The present invention also provides methods for the use of such crystals, for example in structural modeling studies and in rational drug design

The present invention also provides methods for detecting the presence of a lipocalin, such as NGAL in a sample. In one embodiment, such methods comprise (a) contacting with iron a compound capable of binding the lipocalin (e.g. NGAL) and iron (e.g. a compound of the invention), thereby forming a complex between the compound and the iron; (b) contacting the sample with the complex of step (a); and (c) determining the presence of the lipocalin in the sample of step (b) as compared to a sample that does not contain the lipocalin.

In one embodiment, the sample is a biological sample. In a preferred embodiment, the biological sample is urine.

In one aspect of the methods provided by the invention, the compound used in step (a) comprises a bacterial siderophore. In another aspect, the compound comprises a mammalian siderophore. In yet another aspect, the compound comprises a compound of the invention. In one embodiment of the methods of the invention, the compound is conjugated to a detectable label. In another embodiment, the detectable label is a chromophore or a fluorophore.

In one embodiment of the invention, the “determining” performed in step (c) comprises measuring the pH stability of the complex of step (a). In another embodiment, the determining comprises measuring the redox stability of the complex of step (a). In a further embodiment, the determining comprises measuring absorbance of a chromophore or a fluorophore.

### BRIEF DESCRIPTION OF THE FIGURES

FIGS. 1A-1F. Screening of compounds reported in human urine. (A) ⁵⁵Fe binding is detected by mobilization of iron to the front of the paper chromatogram. ⁵⁵FeCl₃ and compounds were spotted together at 10 pmoles and the chromatogram was then developed with water. Various candidate chelators are shown, some with positive result such as catechol and isocitrate and some with negative results, such as myricetin and allantoin. (B) Complexes of NGAL (10 μM), candidate iron chelators (0.5-100 μM) and ⁵⁵Fe (10 μM: 24 nM ⁵⁵Fe+9.76 μM cold Fe) were detected after repetitive washes on a 10 kDa-cutoff filter. ⁵⁵Fe retention was dependent on the addition of candidate chelator in a dose dependent fashion. 2,3-dihydroxybenzoic acid (2,3 DHBA) served as a positive control. (C) Competition for NGAL binding. All samples contained ⁵⁵Fe, apo-NGAL and candidate chelators, and in half the assays a 50 fold molar excess of Fe-Ent (presaturated with cold iron) was present. Apo-NGAL is the negative control, and Ent and 2,3-dihydroxybenzoic acid are positive controls. (D) Fluorescence quenching binding analysis of NGAL and catechols (free ligand, L) or (F) ferric catechols (ferric ligand, FeL₃). Symbols give the fluorescence data at 340 nm and the lines give the calculated fits using a model constructed with two dissociation constants. Note that the presence of Fe^{3|} dramatically changed the affinity for different catechols, (E). Calculated binding constants. Free catechol (L); ferric catechol (FeL₃).

FIGS. 2A-2C. (A) UV-visible spectra of apo-NGAL, Ent:iron and NGAL:Ent:iron (left) and apo-NGAL, catechol:iron, and NGAL:catechol:iron (right). While ligand-metal charge-transfer (LMCT) of Ent:Fe (λₘₐₓ=498 nm), a tris-catecholate compound, was not modified by the addition of NGAL protein (note red coloration (B), 2 left tubes), catechol:iron converted from FeL (blue, λₘₐₓ=575 nm) to the FeL₃ (red, λₘₐₓ=498 nm) species when bound to NGAL, (B, right tubes). (C) Speciation diagram of catechol:iron (10:1). FeL₂ is the predominant complex present at pH 7.4. FeL₃ may be observed in more basic conditions. The speciation diagram was calculated in HySS (Hyperquad Simulation and Speciation) based on the catechol thermodynamic values (Avdeef, A., Sofen, S. R., Bregante, T. L., Raymond, K. N. (1978). Coordination chemistry of microbial iron transport compounds. Stability constants for catechol models of enterobactin. J. Am. Chem. Soc. 100, 5362-5370; Martell, A. E., Smith, R. M. (1976). Critical Stability Constants, Vol 4: Inorganic Ligands (Plenum Press, New York)).

FIG. 3. NGAL binds to both catechol:iron and 4-methylcatechol:iron. The upper two panels show electrostatic surface representations for molecule C, demonstrating positive (blue), neutral (white), and negative (red) charges in the calyx. Individual structures were aligned using pair-wise alignment on all Cα's. Ligands from molecule A (gray) and molecule C (yellow) are shown bound in pocket #1 of the calyx. The middle two panels show a side view of each of the ligands comparing molecule A and molecule C. Catechol (Middle left) shows a rotation of 55 degrees towards the outside of the protein. 4-methylcatechol (Middle right) has a rotation of 10 degrees. Hydroxyl groups facing out of the calyx are potentially protonated or have been oxidized to form a semi-quinone species. Iron is shown in orange for molecule A and yellow for molecule C in both Top and Middle Panels. 2Fo-Fc electron density map (Bottom) of molecule A for catechol (Bottom left) and 4-methylcatechol (Bottom right) contoured at 1 sigma. Waters are shown in red, chloride in green, iron in orange, and the molecule in gray.

FIGS. 4A-4F. Formation and trafficking of the NGAL complex in vivo. (A) NGAL and ¹⁴C-catechol:Fe were introduced separately, and 5 minutes later, serum was harvested to determine whether a complex had formed in vivo. When both components were introduced, gel filtration demonstrated a NGAL:catechol complex, whereas the introduction of catechol alone showed a different pattern of elution. The NGAL:catechol complex migrated with an authentic standard and with immunoreactive NGAL (immunoblot of column fractions in B). Molecular weight standards are indicated. (C) Recovery of ¹⁴C-catechol or NGAL:¹⁴C-catechol complexes in different organs. Whereas free ¹⁴C-catechol was rapidly cleared (>10 min) by different organs, NGAL:¹⁴C-catechol complex was captured by the kidney (NGAL:¹⁴C-Catechol vs ¹⁴C-catechol at 20 min, p=0.0036; at 3 hours, p=0.0217) in excess of the liver at 3 hours (p=0.044). (D) Recovery of citrate:⁵⁵Fe or NGAL:catechol:⁵⁵Fe in different organs. Whereas citrate:⁵⁵Fe locates predominantly to liver, NGAL:catechol:⁵⁵Fe locates predominately to the kidney at 3 hours. The data were presented as mean±Std. (E, F) The distribution of ⁵⁵Fe was visualized by radioautography. ⁵⁵Fe was captured by the proximal tubule when complexed with NGAL:catechol, but much less iron was found in the kidney when it was introduced as a citrate complex. Note the black silver grains particularly at the apical surface of the proximal tubules (*). Both experimental samples were processed together and exposed for 1 month to Ilford emulsion.

FIGS. 5A-5B. NGAL effectively chelates iron. (A). Various catechols (45 μM) reduced ferric iron (15 μM) to ferrous iron (Fe³⁺=>Fe²⁺), which we detected with phenanthroline. The addition of stoichiometric quantities of NGAL (15 μM) however blocked the reaction. (B) Conversion of HPF to fluorescein occurs in the presence of catechol, ferric iron and H₂O₂, but the addition of NGAL blocked the reaction. 0-sulfonation inactivated the participation of catechol in redox cycling. Fluorescein (F) was not affected by the addition of NGAL.

FIGS. 6A-6C. Release of ligands from NGAL by acidification. (A) Fluorescence titration of NGAL with ferric catechol complexes. Fluorescence was quenched by ligand binding. Upon acidification, the ligands were released and fluorescence returned to baseline. Subsequent basification, where relevant, caused rebinding. Note that NGAL:pyrogallol and 2,3-dihydroxybenzoic acid complexes required much lower pH for dissociation. (B) Release of ⁵⁵Fe from different NGAL:catecholate complexes by low pH washes on a 10 KDa microcon. For this comparison, the retention of iron at pH7.0 was defined as 100%. (C) Capture of ⁵⁵Fe from the NGAL:catechol:⁵⁵Fe complex by kidney stromal cells in vitro. ⁵⁵Fe uptake was inhibited at 4° C. or by bafilomycin, an inhibitor of the vacuolar H⁺ATPase. Data were presented as mean±Std.

FIG. 7. Low Molecular Weight Urine (<3K) Mobilizes ⁵⁵Fe³⁺ in Paper Chromatography. Urine contains small molecules that bind iron. Low molecular weight urine samples (<3 KDa) and Fe³⁺ were spotted on a paper chromatogram. The chromatogram was then developed in water. Fe³⁺ was mobilized by the urine sample in a dose dependent fashion. AKI-acute kidney injury; CKD-chronic kidney disease.

FIG. 8. Low Molecular Weight Urine (<3K) Substitutes for Ent and Permits the Retention of ⁵⁵Fe³⁻ by NGAL. Urine contains compounds which enhance the association of iron and NGAL. ⁵⁵Fe³⁺ was combined with apo NGAL, or apo NGAL with either Ent or low molecular weight human or mouse urine (100 μg; <3 KDa), or ethylacetate or aqueous extracts (100 μg) of urine at pH7.0. The NGAL:urine:iron complexes were then washed on a 10K microcon. ⁵⁵Fe³⁺ retention depended on the cofactors supplied by bacteria or by urine. Iron retention by apo NGAL differed significantly from apo NGAL combined with Ent (p=0.021), human urine (p=0.034), mouse urine (p=0.0008), or an ethylacetate extract of human urine (p=0.0007).

FIG. 9. Stable Association of Catechol:⁵⁵Fe with NGAL in Repetitive Washes. NGAL:catechol:⁵⁵Fe complex was repetitively washed on a 10K filter, but the catechol complex retained iron to the same extent as the proven NGAL ligands, Ent and 2,3-dihydroxybenzoic acid (not shown). A representative experiment is shown.

FIG. 10. Stable Association of Catechol: ⁵⁵Fe with NGAL in Gel Filtration. Rapid gel-filtration assay demonstrated that ⁵⁵Fe associates with NGAL in the presence of catechol. Apo-NGAL is a negative control and apo-NGAL Ent serves as a positive control. Note that some free enterochelin elutes with the protein fraction due to its molecular weight (719 Da), whereas free catechol (110 Da) is excluded.

FIG. 11. Relative Position of Catechol and 4-methylcatechol in Crystal Molecule A and C. Relative position of catechol and 4-methylcatechol in molecule A (left) and molecule C (right). In molecule A, there is a rotation of 120 degrees, and 4-methylcatechol shifts up approximately 1.4 Å towards the outside of the calyx. In molecule C, the rings rotate 80 degrees and 4-methylcatechol shifts up approximately 1 Å towards the outside of the calyx. In both molecules A and C, iron is shifted more towards the center of the calyx for 4-methyl-catechol (iron=orange) as opposed to catechol (iron=yellow).

FIG. 12. Superimposition of Catechol and Bacterial Siderophores Ent and Carboxymycobactin. Superposition of ligands from previous NGAL structures: catechol (left) and 4-methylcatechol (right). Catechol:iron is shown in light yellow (molecule A) and dark yellow (molecule C), catechol rings from Ent are shown in light green (molecule A) and dark green (molecule C), 4-methylcatechol:iron is shown in light gray (molecule A) and dark gray (molecule C), phenol oxazoline groups from Cmb (1X89) are shown in light blue (molecule A) and dark blue (molecule C). For clarity only the iron binding portion of the ligands are shown from each structure.

FIG. 13. Presence of Chloride Atoms in the NGAL Pocket. Example of chloride atoms in the NGAL calyx. Shown is an electrostatic surface representation of NGAL bound to Fe-catechol. The electrostatic surface shows positive (blue), neutral (white), and negative (red) charges of the calyx. Chloride atoms (green) are found bound in the calyx of several of the structures, likely compensating for the positive charge of the Fe(III) atom (orange).

FIG. 14. Conversion of tyrosine to catechol. Incubation with intestine overnight resulted in conversion of ³H-tyrosine into a compound migrating with unlabeled catechol (black line, *fraction 25-27, C-18 HPLC analysis) as well as a second metabolite (fraction 9-11); methylation of the organ extract (bright blue) abolished these peaks. Intestine and lung tissue were able to convert tyrosine. The authenticity of the peak was established by its mobility (TLC), and its mobility after dimethylation (TLC), compared with authentic catechol.

FIG. 15. Identification of Catechol in Human Urine; Addition of Standards. Urine contains small molecules that bind iron and NGAL. The most active fraction (urine EtOAc extract) contained catechol (retention time: 25.5-26 min) as demonstrated by the addition of authentic catechol. HPLC-UV 216 nm, 274 nm; C-18 column.

FIG. 16. Demonstration of catechol in ethylacetate extract of urine using ESI Mass. Note catechol mass [M-H]⁻, presumptive dimer [2M-H]⁻ and solvent complexes.

FIG. 17 is a graph of a UV spectrum of the active compounds-Iron-Ngal complex.

FIG. 18 is a dose-response curve of catechol (GB1-56-3) and 2, 3-Dihydroxybenzoic acid (DHBA, GB1-49-1).

FIG. 19 is a bar graph showing the amount of 55Fe retained by a 10 kDa filter after four times washing of catechol and enterochelin.

FIG. 20 is a bar graph showing the amount of 55Fe retained by a 10 kDa filter after three times washing of catechol and DHBA.

FIG. 21 is a UV spectrum of different forms DHBA, showing there are Ngal-DHBA-iron complexes formed (500-700 nm wavelength).

FIG. 22 is a UV spectrum of different forms of Catechol, showing there are Ngal-catechol-iron complexes formed (500-700 nm wavelength).

FIG. 23 is a bar graph depicting Ngal:sideophore:Fe binding activity, wherein catechol and DHBA derivatives' Ngal-55Fe binding activity is demonstrated. The methyl or sulfate substitute on the hydroxyl group makse catechol and DHBA derivatives lose their activity.

FIG. 24 is a scatchard analysis (FIG. 11A) on equilibrium binding of 14C catechol (0.3-30 μM, in a tris solution, pH 7.4). FIG. 11B depicts a different Catechol form bound 55Fe counts at various catechol concentrations.

FIG. 25 is a scatchard analysis (FIG. 12A) on equilibrium binding of 14C catechol (0.3-30 μM, in a tris solution, pH 5.5). FIG. 12B depicts a different Catechol form bound 55Fe counts at various catechol concentrations.

FIG. 26 is a standard curve of catechol HPLC quantification (FIG. 13A). FIG. 13B is a table of monitored UV wavelength, mass, and retention times of catechol.

FIG. 27 is graphs of HPLC curves of urine EtOAc extracts with different concentrations of standard catechol added. FIG. 27A, GB1-96-2 20 μl; FIG. 27B, GB1-96-8 20 μl CONCENTRATED GB1-96-2 2 TIMES; FIG. 27C, GB1-96-3: 30 μl (INCLUDING 0.07 5 μg STANDARD CATECHOL)-0.02 μg; FIG. 27D, GB1-96-4 20 μl 0.05 μg; FIG. 27E, GB 1-96-9 20 μl-0.2 μg.

FIG. 28 is a diagram of the synthesis of catechol sulfate.

FIG. 29 is a reproduction of Silical gel TLC developed by Dichlorlmethane: MeOH:H₂O=5:1:0.1, and colored by 12. Catechol sulfate dissolved in methanol can decompose in pH≦6 when incubated for 1 hour, while kept stable in different urine even incubated for 48 hours.

FIG. 30 is a bar graph of silica chromatography showing 55Fe binding to mouse urine fractions. Fr 1, 100% ethyl acetate; Fr 2, 77% ethyl acetate/23% methanol; Fr 3, 62% ethyl acetate/38% methanol; Fr 4, 50% ethyl acetate/50% methanol; Fr 5, 100% methanol.

FIG. 31 is a bar graph of silica chromatography showing 55Fe binding to dog urine fractions. Fr 1, 100% ethyl acetate; Fr 2, 77% ethyl acetate/23% methanol; Fr 3, 62% ethyl acetate/38% methanol; Fr 4, 50% ethyl acetate/50% methanol; Fr 5, 100% methanol.

FIG. 32 is a bar graph of silica chromatography showing 55Fe binding to human urine fractions. Fr 1, 100% ethyl acetate; Fr 2, 77% ethyl acetate/23% methanol; Fr 3, 62% ethyl acetate/38% methanol; Fr 4, 50% ethyl acetate/50% methanol; Fr 5, 100% methanol.

FIG. 33 is a reproduction of non-limiting examples of siderophores in solution (NGLA:ENT, NGAL, and NGAL-CA; left panel) and in crystal form (right panel). The red crystals, NGAL: rosmarinic acid, a type of catechol, is depicted in the bottom image of the right panel.

FIG. 34 is a diagram of a metabolic flow chart depicting potential synthetic pathways of catechol and catechol derivatives.

FIG. 35 is a mass spectrometry chromatogram of catechol and DHBA in the urine. LCMS detected catechol and DHBA from Ether extract of urine from patients.

FIG. 36 is a reproduction of ESI MS spectrum of Catechol (FIG. 23A) and DHBA (FIG. 23B).

FIG. 37 is a reproduction of LCMS spectrums of Catechol (FIG. 24A), DHBA (FIG. 24B), and Catechol+DHBA (FIG. 24C) detected in the ether exract of urine.

FIG. 38 is a bar graph depicting Ngal-iron binding activity of urine fractions.

FIG. 39. General form of a dipstick is shown on the left: nitrocellulose membrane with immobilized secondary capture layers, and attached conjugate pad (with gold nanoparticles conjugated to a primary capture moiety). Exemplary embodiments: (C) competitive: NGAL in urine binds to gold-nanoparticle conjugated to siderophore analog; excess conjugates bind to NGAL immobilized on a capture line; optionally, a control line with anti-NGAL antibody can be added. Situations with low, high and very high concentrations of NGAL in urine are shown. (NC) non-competitive: NGAL in urine binds to gold-nanoparticles conjugated to siderophore analogs; this complex is bound on capture layer by anti-NGAL antibodies; optionally, a control strip with anti-siderophore analog can be used to capture excess gold-siderophore analog conjugates. Situations with low, high and very high concentrations of NGAL in urine are shown, with two capture lines.

### DETAILED DESCRIPTION

Iron (Fe) is an essential element for almost all life forms, including humans, where iron is present in all cells and carries out vital functions for example as a carrier of oxygen (in the form of hemoglobin) from the lungs to the tissues, and in enzymatic reactions in various tissues. Humans are equipped with proteins, enzymes and metabolic processes which function to maintain iron concentrations at appropriate levels. If iron levels are not properly regulated, iron can become toxic by catalyzing redox reactions, resulting in the formation of free radicals which cause cell death. Due to the potentially toxic nature of free iron in cells, iron is transported in the body in the form of complexes where it is bound or chelated to proteins (such as transferrin) or other molecules which reduce the toxic potential of iron.

Iron is present in the environment in forms that are largely insoluble (ferric iron or Fe³⁺), thus limiting its biologic availability (or bioavailability). To be useful in biological processes, iron must be in a soluble form (ferrous iron or Fe²⁺) that can be efficiently absorbed by the body. Chelation of iron by proteins helps keep dietary iron soluble, however, while total dietary iron in humans usually exceeds requirements, the bioavailability of iron in the diet is limited.

Iron deficiency anemia is the most common form of anemia. About 20% of women, 50% of pregnant women, and 3% of men are iron deficient. The causes of iron deficiency are too little iron in the diet, poor absorption of iron by the body, and loss of blood. Moreover, examples of diseases associated with anemia include chronic kidney disease, cancer, HIV/AIDS, hepatitis, autoimmune diseases, and cardiovascular disease. Oral iron supplements (ferrous sulfate) and intravenous (IV) or intra-muscular iron injections are available. However, these are associated with toxicities.

The present invention is based, in part, on the discovery of a family of mammalian catechol-related iron-binding compounds or “siderophores” that bind with high affinity to lipocalin proteins, and that are able to bind to, transport, and release iron in vivo. These catechol-related compounds, and compositions containing such a catechol-related compound and a lipocalin, may be used as iron chelators and/or iron donors and may be useful in the treatment of various conditions, diseases and disorders associated with excessive iron levels and/or iron deficiency.

### Iron-Binding Siderophores

Siderophores are high affinity iron (e.g. Fe³⁺) binding compounds.

The vast majority of siderophores known are produced by bacteria. Bacteria release siderophores into the surrounding environment for the purpose of scavenging or chelating iron and transporting the iron to the bacteria—a process necessary for survival of bacteria. Siderophores that are known in the art include, but are not limited to enterochelin, TRENCAM, MECAM, TRENCAM-3,2-HOPO, parabactin, carboxymycobactin, fusigen, triacetylfusarinine, feriichrome, coprogen, rhodotorulic acid, ornibactin, exochelin, ferrioxamine, desferrioxamine B, aerobactin, ferrichrome, rhizoferrin, pyochelin, pyoverdin. The structures of these compounds are disclosed in Holmes et al. 2005 and Flo et al., 2004, the contents of which are hereby incorporated by reference.

Several of the above siderophores are known to bind to lipocalins, including NGAL, and complexes of these siderophores and lipocalins are known to be able to sequester iron (see for example, Holmes et al. 2005 and Flo et al., 2004, Goetz et al, 2002, and Mori, et al., (2005), “Endocytic delivery of lipocalin-siderophore-iron complex rescues the kidney from ischemia-reperfusion injury.” J. Clin Invest. 115, 610-621).

The present invention is based, in part, on the discovery of a new family of mammalian catecholate iron-binding “siderophore” compounds. These compounds may be referred to interchangeably herein as “the compounds of the invention,” the “catcehol-related compounds of the invention,” the “catceholate compounds of the invention” or the “sideropores of the invention.”

The compounds of the invention bind with high affinity to lipocalin proteins, such as neutrophil gelatinase-associated lipocalin (“NGAL”), and complexes containing the compounds of the invention and a lipocalin are able to bind to, transport, and release iron in vivo. These catechol-related compounds, and complexes containing such a catechol-related compound and a lipocalin, may be used as iron chelators and/or iron donors and may be useful in the treatment of various conditions, diseases and disorders associated with excessive iron levels and/or iron deficiency.

Compounds of the invention include those dscribed by Formula I, Formula I(a), Formula 1(b), Formula II, and Formula III, the structures of which are provided below.

Accordingly, in one embodiment, the invention provides compounds of Formula I:

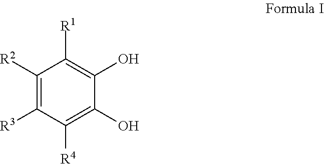

an pharmaceutically acceptable salts or hydrates thereof, wherein:
R¹ is H, halogen, OR⁵, N(R⁵)₂, NO₂, N₃, CN, CO₂R⁵, —C(═O)N(R⁵)₂, S(R⁵), SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl, or R⁶;
R² is H, halogen, OR⁵, N(R⁵)₂, NO₂, N₃, CN, CO₂R⁵, —C(═O)N(R⁵)₂, S(R⁵), SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl, a carbonyl forming an ester with a hydroxyl at the 3-position of a catechol, or R⁶;
R³ is H, halogen, OR⁵, N(R⁵)₂, NO₂, N₃, CN, CO₂R⁵, —C(═O)N(R⁵)₂, S(R⁵), SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl, catechol-4-yl,

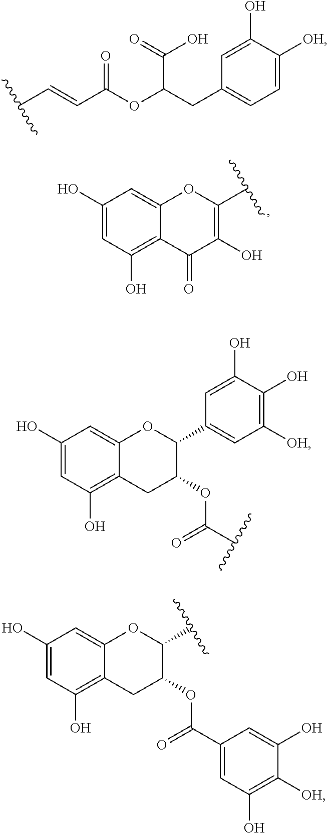

or R⁶, wherein the C₁₋₆ alkyl is optionally substituted with

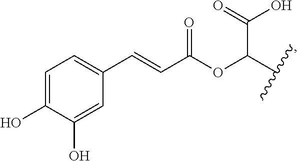

and the catechol-4-yl is optionally substituted with a 5-CO₂R⁵, a 3-OR⁵, or both, or two compounds of formula I are bonded together at the R³ positions, or two compounds of formula I are bonded together at the R³ positions where R² is —CO₂R⁵ and R⁴ is —OR⁵, or two compounds of formula I are bonded together at the R³ positions where R² is —CO₂R⁵ and R⁴ is —OR⁵ and the R² acyl groups form esters with the R⁴ hydroxyl group of the other compound;
R⁴ is H, halogen, OR⁵, N(R⁵)₂, NO₂, N₃, CN, CO₂R⁵, —C(=O)N(R⁵)₂, S(R⁵), SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl, a hydroxyl forming an ester with a carbonyl at the 5-position of a catechol, or R⁶;
each R⁵ is independently H or C₁₋₆ alkyl;
R⁶ is

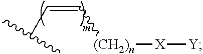

X is —NR⁵—, —O—, —C(═O)O—, or —C(O)NR⁵—;
Y is H, —C(═O)R⁵, C₁₋₆-alkyl, C₃₋₁₀ aryl, C₃₋₁₀ cycloalkyl, or C₁₋₆ heterocyclyl;
m is an integer ranging from 0 to 2; and
n is an integer ranging from 0 to 4.

In one embodiment, the compound of Formula I is not dihydroxybenzoic acid or N-dihydroxybenzoyl-serine.

In one embodiment, the invention provides compounds of Formula Ia:

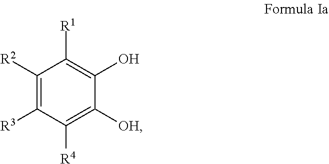

and pharmaceutically acceptable salts or hydrates thereof, wherein:
R¹ is H or OR⁵;
R² is H or carbonyl forming an ester with a hydroxyl at the 3-position of a catechol;
R³ is H, C₁₋₆ alkyl, catechol-4-yl,

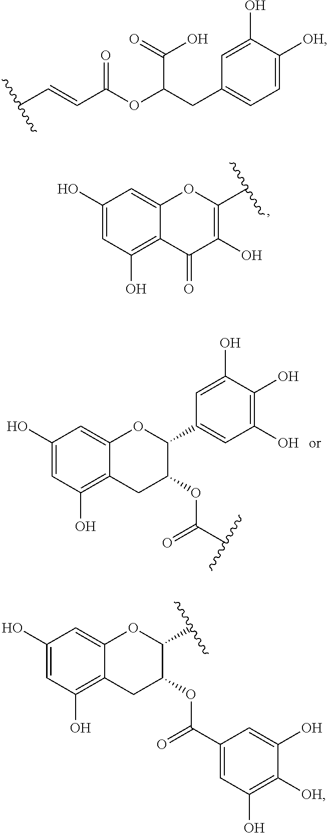

wherein the C₁₋₆ alkyl is optionally substituted with

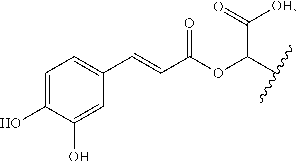

and the catechol-4-yl is optionally substituted with a 5-CO₂R⁵, a 3-OR⁵, or both;
R⁴ is H, C₁₋₆ alkyl, OR⁵, CO₂R⁵, or hydroxyl forming an ester with a carbonyl at the 5-position of a catechol; and
each R⁵ is independently H or C₁₋₆ alkyl.

In one embodiment, C₁₋₆ alkyl is methyl.

In one embodiment, R¹ is H.

In another embodiment, R¹ is OH.

In one embodiment, R² is H.

In one embodiment, R³ is H.

In another embodiment, R³ is methyl.

In one embodiment, R⁴ is H.

In another embodiment, R⁴ is methyl.

In another embodiment, R⁴ is OH.

In one embodiment, R⁵ is H.

In another embodiment, R⁵ is methyl.

In one embodiment, C₁₋₆ heterocyclyl is shikimic acid.

In one embodiment, the compound of Formula Ia is not dihydroxybenzoic acid or N-dihydroxybenzoyl-serine.

In another one embodiment, the invention provides compounds of Formula Ib:

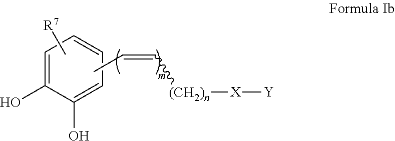

and pharmaceutically acceptable salts or hydrates thereof, wherein:

R⁷ is H, halogen, OH, —O—C₁₋₆ alkyl, NH₂, —NH—C₁₋₆-alkyl, —N(C₁₋₆-alkyl)₂, NO₂, N₃, CN, CO₂H, —C(═O)NH₂, SH, —S—C₁₋₆ alkyl, SO₃H, SO₂NH₂, C₁₋₆-alkyl, C₃₋₁₀ aryl, —O—C₃₋₁₀ aryl, —NH—C₃₋₁₀ aryl, —N(C₁₋₆-alkyl)-C₃₋₁₀ aryl, or —S—C₃₋₁₀ aryl;
X is —NH—, —O—, —C(═O)O—, or —C(═O)NH—;
Y is H, —C(═O)-C₁₋₆ alkyl, C₁₋₆ alkyl, C₃₋₁₀ aryl, C₃₋₁₀ cycloalkyl, or C₁₋₆ heterocyclyl;
m is an integer ranging from 0 to 2; and
n is an integer ranging from 0 to 4.

In one embodiment, the compound of Formula Ib is not dihydroxybenzoic acid or N-dihydroxybenzoyl-serine.

In another embodiment, the invention provides compounds of Formula II:

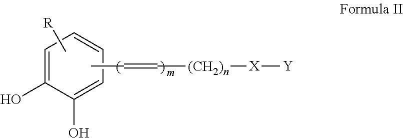

and pharmaceutically acceptable salts and hydrates thereof, wherein
R is H, halogen, OH, NH₂, NO₂, N₃, CN, CO₂H, CONH₂, SH, SO₂OH, SO₂NH₂, alkyl, alkyloxy, alkylamino, alkylthio, aryl, aryloxy, arylamino, or arylthio;
X is N, O, C(O)O, or C(O)NH;
Y is H, acyl, alkyl, aryl, cycloalkyl, or heterocyclyl;
m is an integer ranging from 0 to 2; and
n is an integer ranging from 0 to 4.

In one embodiment, the compound of Formula II is not dihydroxybenzoic acid or N-dihydroxybenzoyl-serine.

In another embodiment, the invention provides compounds of Formula III:

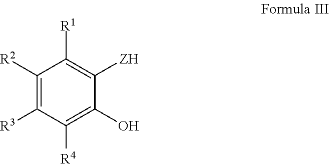

and pharmaceutically acceptable salts or hydrates thereof, wherein:
Z is NH, NMe, O, or CO_{2;}
R¹ is H, halogen, OR⁵, N(R⁵)₂, NO₂, N₃, CN, CO₂R⁵, —C(═O)N(R⁵)₂, S(R⁵), SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-SR⁵, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl, or R⁶, or —CO₂—C₁₋₆-alkyl wherein the C₁₋₆ alkyl is substituted with OR⁵, N(R⁵)₂, NO₂, N₃, CN, CO₂R⁵, —C(═O)N(R⁵)₂, S(R⁵), SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl,;
R² is H, halogen, OR⁵, N(R⁵)₂, NO₂, N₃, CN, CO₂R⁵, —C(═O)N(R⁵)₂, S(R⁵), SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-SR⁵, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl, a carbonyl forming an ester with a hydroxyl at the 3-position of a catechol, or R⁶;

R³ is H, halogen, OR⁵, N(R⁵)₂, NO₂, N₃, CN, CO₂R⁵, —C(═O)N(R⁵)₂, S(R⁵), SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-SR⁵, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl, catechol-4-yl, C₂₋₆ alkenyl

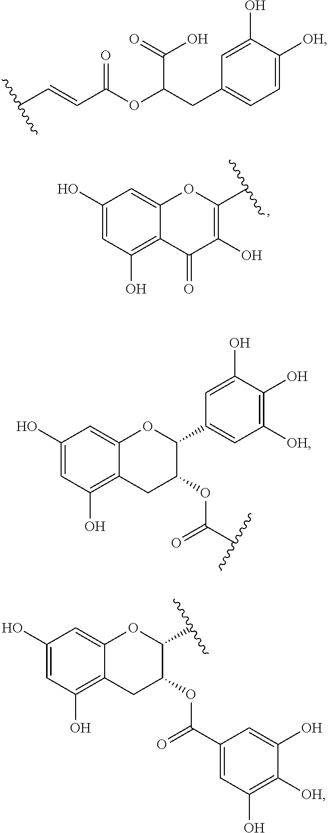

or R⁶, wherein the C ₁₋₆ alkyl is optionally substituted with OR⁵, N(R⁵)₂, NO₂, N₃, CN, CO₂R⁵, —C(═O)N(R⁵)₂, S(R⁵), SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-CO₂R⁵, C₃₋₁₀ aryl, —O—C₃₄₀ aryl, —NR⁵—C₃₋₁₀ aryl, —S—C₃₋₁₀ aryl or

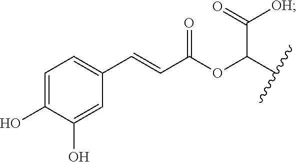

wherein the C₂₋₆ alkenyl is optionally substituted with OR⁵, N(R⁵)₂, NO₂, N₃, CN, CO₂R⁵, —C(═O)N(R⁵)₂, S(R⁵), SO₃(R⁵)₂, C₁₋₆-alkyl, C₁₋₆-alkyl-OR⁵, C₁₋₆-alkyl-N(R⁵)₂, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl, or quinic acid; and wherein the catechol-4-yl is optionally substituted with a 5-CO₂R⁵, a 3-OR⁵, or both, or two compounds of formula I are bonded together at the R³ positions, or two compounds of formula I are bonded together at the R³ positions where R² is —CO₂R⁵ and R⁴ is —OR⁵, or two compounds of formula I are bonded together at the R³ positions where R² is -CO₂R⁵ and R⁴ is —OR⁵ and the R² acyl groups form esters with the R⁴ hydroxyl group of the other compound;
R⁴ is H, halogen, OR⁵, N(R⁵)₂, NO₂, N₃, CN, CO₂R⁵, —C(═O)N(R⁵)₂, SR⁵, SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-SR⁵, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl, a hydroxyl forming an ester with a carbonyl at the 5-position of a catechol, or R⁶;
each R⁵ is independently H or C₁₋₆ alkyl;
R⁶ is

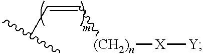

X is —NR⁵—, —O—, —C(═O)O—, or —C(O)NR⁵—;
Y is H, —C(═O)R⁵, C₁₋₆-alkyl, C₃₋₁₀ aryl, C₃₋₁₀ cycloalkyl, or C₁₋₆ heterocyclyl;
m is an integer ranging from 0 to 2; and
n is an integer ranging from 0 to 4.

In one embodiment, Z is O.

In another embodiment, Z is NH.

In another embodiment, Z is CO₂.

In one embodiment, R¹-R⁴ are H.

In another embodiment, at least one of R¹-R⁴ is not H.

In one embodiment, R⁵ is H.

In one embodiment, the compound of Formula III is not dihydroxybenzoic acid or N-dihydroxybenzoyl-serine.

In some embodiments, the compounds of the invention include catehcol, 3-methylcatechol, 4-methylcatechol, rosmarinic acid, myricetin, epigallocatechin gallate, pyrogallol, 2,3-dihydroxybenzoic acid, 3,4-dihydroxybenzoic acid and ellagic acid. The structures of these compounds are provided in Table A.

In preferred embodiments, the compounds of the invention include catehcol, 3-methylcatechol, 4-methylcatechol, rosmarinic acid, myricetin, epigallocatechin gallate, pyrogallol, and ellagic acid. The structures of these compounds are provided in Table A.

TABLE A
High-affinity NGAL binding catechols

| Compound | Structure |
|---|---|
| Catechol | 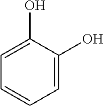 |
| 3-methylcatechol | 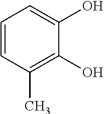 |
| 4-methylcatechol | 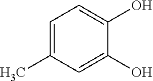 |
| Rosmarinic acid | 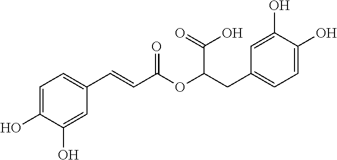 |
| Myricetin | 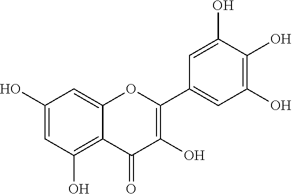 |
| (−) Epigallo- catechin gallate | 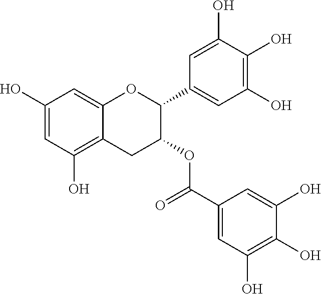 |
| Benzene 1,2,3 Trial (Pyrogallol) | 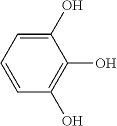 |
| 2,3-Dihydroxy- benzoic acid | 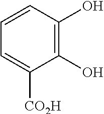 |
| Ellagic acid | 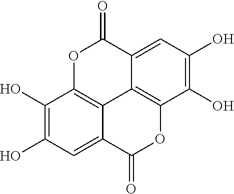 |

In another embodiment, the invention provides a compound selected from catechol, guaiacol, 1,2-dimethoxybenzene, catechol cyclic sulfonate, catechol sulfonate sodium, 3-methylcatechol, 4-methylcatechol, 3,4-dihydroxy-DL-phenylalanine, dihydroxyphenyl alanine (L-DOPA), DL-norepinephrine.HCl, caffeic acid, ferulic acid, caffeic acid phenethyl ester, rosmarinic acid, chlorogenic acid, 5-hydroxydopamine, 6-hydroxydopamine, myricetin, (−)epigallocatechin gallate, benzene 1,2,3 triol (pyrogallol), 2,3-dihydroxybenzoic acid, 2,3-dimethoxybenzoic acid, 3-hydroxyanthranilic acid, 3,4-dihydroxybenzoic acid, salicylic acid, ellagic acid, homogentisic acid, gentistic acid, 3-hydroxy-DL-kynurenine, L-phenylalanine, N-acetyl-DL-A-phenylalanine, L-tryptophan, 5-hydroxytryptophan, 5-hydroxy-indoleacetic acid, uracil, orotic acid, DL-dihdroorotic acid, nicotinic acid, 2,3-pyridinedicarboxylic acid, pyridoxal, 4-pyridoxic acid, 2-furoylglycine, porphobilinogen, glycyl-L-proline, allantoin, bilirubin, biliverdin HCl, urobilin HCl, porphyrin, protoporphyrin IX, flavin adenine dinucleotide (FAD), FADH, nicotinamide adenine dinucleotide phosphate (NADP), NADPH, NAD, NADH, folic acid, maleic acid, citric acid sodium, succinic acid, 5-aminolevulinic acid, cis-aconitic acid, and isocitric acid, or a pharmaceutically acceptable salt or hydrate thereof. The structures of such compounds are provided in the Examples section of this application.

In another embodiment, the invention provides a compound selected from catechol, 3-methylcatechol, 4-methylcatechol, rosmarinic acid, myricetin, epigallocatechin gallate, pyrogallol, 2,3-dihydroxybenzoic acid and ellagic acid or a pharmaceutically acceptable salt or hydrate thereof.

In another embodiment, the invention provides a compound selected from catechol, 3-methylcatechol, 4-methylcatechol, rosmarinic acid, myricetin, epigallocatechin gallate, pyrogallol, and ellagic acid or a pharmaceutically acceptable salt or hydrate thereof.

In yet another embodiment, the invention provides a compound selected from catechol, 3-methylcatechol, 4-methylcatechol, pyrogallol, 2,3-dihydroxybenzoic acid (2,3-DHBA) or 3,4-dihydroxybenzoic acid (3,4-DHBA).

In yet another embodiment, the invention provides a compound selected from catechol, 3-methylcatechol, 4-methylcatechol, or pyrogallol.

In another embodiment, the invention provides a composition comprising a lipocalin and a compound of Formula I or a pharmaceutically acceptable salt or hydrate thereof, and optionally, iron.

In another embodiment, the invention provides a composition comprising a lipocalin and a compound of Formula Ia or a pharmaceutically acceptable salt or hydrate thereof, and optionally, iron.

In another embodiment, the invention provides a composition comprising a lipocalin and a compound of Formula Ib or a pharmaceutically acceptable salt or hydrate thereof, and optionally, iron.

In another embodiment, the invention provides a composition comprising a lipocalin and a compound of Formula II or a pharmaceutically acceptable salt or hydrate thereof, and optionally, iron.

In another embodiment, the invention provides a composition comprising a lipocalin and a compound of Formula III or a pharmaceutically acceptable salt or hydrate thereof, and optionally, iron.

In another embodiment, the invention provides a composition comprising lipocalin and a compound selected from catechol, guaiacol, 1,2-dimethoxybenzene, catechol cyclic sulfonate, catechol sulfonate sodium, 3-methylcatechol, 4-methylcatechol, 3,4-dihydroxy-DL-phenylalanine, dihydroxyphenyl alanine (L-DOPA), DL-norepinephrine.HCl, caffeic acid, ferulic acid, caffeic acid phenethyl ester, rosmarinic acid, chlorogenic acid, 5-hydroxydopamine, 6-hydroxydopamine, myricetin, (-)epigallocatechin gallate, benzene 1,2,3 triol (pyrogallol), 2,3-dihydroxybenzoic acid, 2,3-dimethoxybenzoic acid, 3-hydroxyanthranilic acid, 3,4-dihydroxybenzoic acid, salicylic acid, ellagic acid, homogentisic acid, gentistic acid, 3-hydroxy-DL-kynurenine, L-phenylalanine, N-acetyl-DL-A-phenylalanine, L-tryptophan, 5-hydroxytryptophan, 5-hydroxy-indoleacetic acid, uracil, orotic acid, DL-dihdroorotic acid, nicotinic acid, 2,3-pyridinedicarboxylic acid, pyridoxal, 4-pyridoxic acid, 2-furoylglycine, porphobilinogen, glycyl-L-proline, allantoin, bilirubin, biliverdin HCl, urobilin HCl, protoporphyrin IX, flavin adenine dinucleotide (FAD), nicotinamide adenine dinucleotide phosphate (NADP), NADPH, NAD, folic acid, maleic acid, citric acid sodium, succinic acid, 5-aminolevulinic acid, cis-aconitic acid, and isocitric acid, or a pharmaceutically acceptable salt or hydrate thereof, and optionally, iron.

In another embodiment, the invention provides a composition comprising a lipocalin and a compound selected from catechol, 3-methylcatechol, 4-methylcatechol, rosmarinic acid, myricetin, epigallocatechin gallate, pyrogallol, 2,3-dihydroxybenzoic acid and ellagic acid or a pharmaceutically acceptable salt or hydrate thereof.

In another embodiment, the invention provides a composition comprising a lipocalin and a compound selected from catechol, 3-methylcatechol, 4-methylcatechol, rosmarinic acid, myricetin, epigallocatechin gallate, pyrogallol, and ellagic acid, or a pharmaceutically acceptable salt or hydrate thereof.

The compounds of the invention, as described above, can be obtained, manufactured or synthesized using any suitable means known in the art. As described in the examples, the compounds of the invention can be obtained from a commercial source. For example, all each of the nine compounds referred to in Table A is commercially available from Sigma-Aldrich. Alternatively, one of skill in the art can synthesize the compounds of the invention, for example using published synthetic protocols. Alternatively, one of skill in the art can isolate the compounds of the invention from a suitable natural source, for example from urine—as described in the Examples, or from a cell type or cell culture that normally produces the compounds of the invention.

### Lipocalins

The present invention provides mammalian iron-binding catecholate compounds that bind with high affinity to lipocalin proteins, and also provides compositions that contain a compound of the invention and a lipocalin protein. Complexes containing the catechol-related compounds of the invention and a lipocalin, may be used as iron chelators and/or iron donors and may be useful in the treatment of various conditions, diseases and disorders associated with excessive iron levels and/or iron deficiency.

Lipocalins are proteins that generally transport small organic molecules. There are about 20 known proteins in the lipocalin family. While the ligands for many members of this family have been determined (retinal binding protein, purpurin, and rat epididymal RBP bind retinoids, the major urinary binding proteins bind pheromones, astaxanthin binds colorants, and nitrophorins and α1-microglobulin bind heme; Akerstrom B, Flower D R, Salier J P. (2000) Lipocalins: unity in diversity. Biochim Biophys Acta. 1482, 1-8), the identification of ligands for other family members is still ongoing.

Any lipocalin protein, or homolog, variant, derivative, fragment, or mutant thereof, that binds to a compound of the invention and/or is able to form a complex containing iron, a compound of the invention, and a lipocalin protein, homolog, variant, derivative, fragment, or mutant, may be used in accordance with the present invention. One of skill in the art can readily determine whether a given homolog, variant, derivative, fragment, or mutant has the ability to bind to a compound of the invention and/or is able to form a complex containing iron, a compound of the invention, for example using the methods described herein.

In a preferred embodiment, the lipocalin protein is a mammalian NGAL protein, or a homolog, variant, derivative, fragment, or mutant thereof, that has the ability to bind to a compound of the invention, and/or is able to form a complex containing iron, a compound of the invention, and the NGAL protein, homolog, variant, derivative, fragment, or mutant. One of skill in the art can readily determine whether a given homolog, variant, derivative, fragment, or mutant has the ability to bind to a compound of the invention and is able to form a complex containing iron and a compound of the invention, for example using the methods described herein. Unless stated otherwise, the term “NGAL”, as used herein, refers to all such mammalian NGAL proteins, homologs, variants, derivatives, fragments, or mutants thereof. In preferred embodiments the NGAL protein is a human NGAL protein.

Neutrophil Gelatinase Associated Lipocalin or “NGAL” is also referred to in the art as human neutrophil lipocalin, siderocalin, a-micropglobulin related protein, Scn-NGAL, lipocalin 2, 24p3, superinducible protein 24 (SIP24), uterocalin, and neu-related lipocalin.

In certain embodiments, the NGAL protein used according to the present invention has an amino acid sequence as defined by one of the following GenBank accession numbers, NP_{—}005555, CAA67574, P80188, AAB26529, P11672, P30152, AAI132070, AAI132072, AAH33089, AAB72255, and CAA58127, or is a homolog, variant, derivative, fragment, or mutant thereof that has the ability to bind to a compound of the invention (for example by virtue of being structurally conserved in the region of “binding pocket 1”), and/or has at least 80% sequence identity, e.g., 85%, 90%, 95%, 98% or 99% sequence identity, with one of the above sequences.

For example, in the NGAL protein may have one of the following sequences:

Sequence Table
SEQ ID NO. 1 (Human NGAL; AAB26529)

| | |
|---|---|
| 1 | edstsdlipa pplskvplqq nfqdnqfqgk wyvvglagna ilredkdpqk myatiyelke |
| 61 | dksynvtsvl frkkkcdywi rtfvpgcqpg eftlgniksy pgltsylvrv vstnynqham |
| 121 | vffkkvsqnr eyfkitlygr tkeltselke nfirfskslg lpenhivfpv pidqcidg |

SEQ ID NO. 2 (Human NGAL C87S)

| | |
|---|---|
| 1 | edstsdlipa pplskvplqq nfqdnqfqgk wyvvglagna ilredkdpqk myatiyelke |
| 61 | dksynvtsvl frkkkcdywi rtfvpgsqpg eftlgniksy pgltsylvrv vstnynqham |
| 121 | vffkkvsqnr eyfkitlygr tkeltselke nfirfskslg lpenhivfpv pidqcidg |

SEQ ID NO. 3 (Human NGAL precursor; NP_005555)

| | |
|---|---|
| 1 | mplgllwlgl allgalhaqa qdstsdlipa pplskvplqq nfqdnqfqgk wyvvglagna |
| 61 | ilredkdpqk myatiyelke dksynvtsvl frkkkcdywi rtfvpgcqpg eftlgniksy |
| 121 | pgltsylvrv vstnynqham vffkkvsqnr eyfkitlygr tkeltselke nfirfskslg |
| 181 | lpenhivfpv pidqcidg |

SEQ ID NO. 4 (Human NGAL precursor C127S)

| | |
|---|---|
| 1 | mplgllwlgl allgalhaqa qdstsdlipa pplskvplqq nfqdnqfqgk wyvvglagna |
| 61 | ilredkdpqk myatiyelke dksynvtsvl frkkkcdywi rtfvpgsqpg eftlgniksy |
| 121 | pgltsylvrv vstnynqham vffkkvsqnr eyfkitlygr tkeltselke nfirfskslg |
| 181 | lpenhivfpv pidqcidg |

In some embodiments, the NGAL protein has the sequence of SEQ ID NO. 1, 2, 3, or 4. In preferred embodiments, the NGAL protein has the sequence of SEQ ID NO. 1 or 2, or is a homolog, variant, derivative, fragment, or mutant thereof that has the ability to bind to a compound of the invention (for example by virtue of being structurally conserved in the region of “binding pocket 1”), and/or has at least 80% sequence identity, e.g., 85%, 90%, 95%, 98% or 99% sequence identity, with one of the above sequences.

In other preferred embodiments, the NGAL protein has the sequence SEQ ID NO. 2, which differs from SEQ ID NO. 1 in that amino acid residue 87 is a serine as opposed to a cysteine, or is a homolog, variant, derivative, fragment, or mutant thereof that has the ability to bind to a compound of the invention (for example by virtue of being structurally conserved in the region of “binding pocket 1”), and/or has at least 80% sequence identity, e.g., 85%, 90%, 95%, 98% or 99% sequence identity, with one of the above sequences.

In certain embodiments, the NGAL protein used according to the present invention has an amino acid sequence as defined by of SEQ ID NO.s 3, or 4, which are NGAL precursors, or is a homolog, variant, derivative, fragment, or mutant thereof that, when processed to the mature form, has the ability to bind to a compound of the invention (for example by virtue of being structurally conserved in the region of “binding pocket 1”), and/or has at least 80% sequence identity, e.g., 85%, 90%, 95%, 98% or 99% sequence identity, with one of the above sequences.

The lipocalins described herein, such as NGAL, can be obtained from any suitable source or produced by any suitable method known in the art. For example, in preferred embodiments, the lipocalins, such as NGAL, are recombinantly produced. Methods for the recombinant production of proteins are well known in the art. For example, a nucleotide sequence encoding the desired lipocalin protein, such as NGAL, may be included in an expression vector containg expression control sequences and expressed in, and purified from any suitable cell type, such as bacterial cells or mammalian cells. In preferred embodiments, NGAL proteins are recombinantly produced in and purified from bacterial cells as described in Yang et al., (2002) and/or Goetz et al. (2000) and/or Goetz et al. (2002) and/or Mori et al. (2005), the contents of which are hereby incorporated by reference.

It is known in the art that Expression of the NGAL is massively induced during cell damage. Levels of this NGAL rise 100-1000 fold (to >2 μg/ml or 0.4 μM) during hypoxia/ischemia, cytotoxicity, and sepsis (Barasch, Annals of Internal Medicine, 2008). The protein is found in the blood and in the urine and its source is, by and large, the epithelial organs of the body, with a more minor contribution from neutrophils. Cloning and expression of NGAL recombinantly in bacteria revealed that the protein could sequester a bacterial molecule called enterochelin, a bacterial iron-binding siderophore. Sequestation of the siderophore by NGAL prevented iron's use by bacteria (Strong, Mol Cell, 2002; Nature, 2005). But since NGAL was also massively expressed in the absence of microbial infection, its function in aseptic states has been uncertain. In addition, given that crystal structures were generated by cloning and expression of the protein in bacteria, the question arose as to the existence and/or identity of a natural ligand that binds to mammalian/circulating NGAL.

Some of the ligands for NGAL were identified when the recombinant NGAL protein was subjected to x-ray crystallography which identified the source of the protein's reddish color. Its ligands were organic molecules synthesized by bacteria for the purpose of chelating and acquiring iron. NGAL ligands included bacterial enterochelin (Ent), synthesized by Gram-negative organisms; bacillibactin (BB), from Gram-positive organisms; and carboxymycobactins (Cmb) from mycobacteria (Goetz, D. H., Holmes, M. A., Borregaard, N., Bluhm, M. E., Raymond, K. N., Strong, R. K. (2002). The Neutrophil Lipocalin NGAL Is a Bacteriostatic Agent that Interferes with Siderophore-Mediated Iron Acquisition. Mol. Cell 10, 1033-1043; Holmes, M. A., Paulsene, W., Jide, X., Ratledge, C., Strong, R. K. (2005). Siderocalin (Lcn 2) also binds carboxymycobactins, potentially defending against mycobacterial infections through iron sequestration. Structure 13, 29-41; Abergel, R. J., Wilson, M. K., Arceneaux, J. E. L., Hoette, T. M., Strong, R. K., Byers, B. R., and Raymond, K. N. (2006). “The Anthrax Pathogen Evades the Mammalian Immune System Through Stealth Siderophore Production”. Proc. Natl. Acad. Sci. USA 103, 18499-18503). These siderophores bound to NGAL with affinities high enough to block iron traffic to bacteria. While these data demonstrated a mechanism of iron sequestration by a mammalian NGAL protein, they reflected mechanisms specific to infections (Goetz et al., 2002; Flo, T. H., Smith, K. D., Sato, S., Rodriguez, D. J., Holmes, M. A., Strong, R. K., Akira, S., Aderem, A. (2004). Lipocalin 2 mediates an innate immune response to bacterial infection by sequestrating iron. Nature 432, 917-921).

In the present invention biochemical screens were used to identify a family of catechols that bind iron in the calyx of NGAL. The site of molecular recognition was determined by x-ray crystallography and was found to mimic the sites of interaction of NGAL with bacterial siderophores. Most importantly, while the affinity for catechol was quite low, the presence of iron enhanced this affinity nearly 10⁵ fold, and the color of the iron-complex changed from blue to red. In short, NGAL itself recruited catechol monomers to form L₃Fe ligands, generating a hexadentate iron chelate.

As stated above, NGAL is normally expressed at low levels in different compartments of the body, but a number of stimuli can raise its concentration by orders of magnitude. Activation of Toll-Like Receptors (TLR2 or TLR4) by bacterial ligands such as Lipopolysaccharide (LPS) induce a 1000 fold increase in NGAL message in liver and spleen, and a 10-100 fold increase in serum NGAL protein (Flo et al., 2004), consistent with the finding that NGAL chelates bacterial siderophores. But in addition, non-bacterial stimuli can also induce NGAL expression in different organs. This has been best studied in the kidney, where in the nominal absence of infection, stimuli such as ischemia, urinary obstruction and cytotoxic agents raise NGAL expression in different segments of the nephron by up to 1000 fold in proportion to the stimulus. This occurs not only in rodents, but also in human neonates (J. Barasch, unpublished), children (Mishra J, Dent C, Tarabishi R, Mitsnefes M M, Ma Q, Kelly C, Ruff S M, Zahedi K, Shao M, Bean J, Mori K, Barasch J, Devarajan P. (2005). Neutrophil gelatinase-associated lipocalin (NGAL) as a biomarker for acute renal injury after cardiac surgery. Lancet. 365, 1231-8) and adults (Nickolas, T. L., O'Rourke, M. J., Yang, J., Sise, M. E., Canetta, P. A., Barasch, N., Buchen, C., Khan, F., Mori, K., Giglio, J., Devarajan, P., Barasch, J. (2008). Sensitivity and specificity of a single emergency department measurement of urinary neutrophil gelatinase-associated lipocalin for diagnosing acute kidney injury. Annals Internal Medicine. 148, 810-819).

Given that both bacterial (Flo et al., 2004; Nelson A. L., Barasch, J. M., Bunte, R. M., Weiser, J. N. (2005). Bacterial colonization of nasal mucosa induces expression of siderocalin, an iron-sequestering component of innate immunity. Cell Microbiol. 7, 1404-1417) and non-bacterial stimuli induce NGAL expression, NGAL may serve as a prophylactic to reduce the possibility of bacterial infection in damaged epithelia, before bacterial siderophores are expected to be present. Alternatively, NGAL may serve other physiological functions by binding additional ligands. This hypothesis is supported by structural analyses of NGAL. First, it was found that bacterial enterochelin (Ent) failed to fill the NGAL ligand binding site, or calyx, implicating additional ligands. Second, two proven ligands, Ent and carboxymycobactin were found to be structurally dissimilar, implying that a variety of molecules may occupy the protein's calyx (Holmes et al., 2005). Third, it was found that a related member of the lipocalin superfamily, lipocalinl, could accommodate a variety of siderophores (Fluckinger, M., Haas, H., Merschak, P., Glasgow, B. J., Redl, B. (2004). Human tear lipocalin exhibits antimicrobial activity by scavenging microbial siderophores. Antimicrob Agents Chemother. 48, 3367-3372). Lastly, while the bacterial siderophores are not synthesized by mammalian cells, they are composites of well known functional groups such as hydroxybenzoates and hydroxybenzenes which are found in a variety of compounds in mammalian serum and urine. However, prior to the present invention, no other NGAL ligands had been identified in mice or humans.

Given that NGAL is abundant throughout the urinary system, mouse and human aseptic urine was screened as a source of biomolecules to identify endogenous NGAL ligands that could also serve as iron chelators and/or donors. Initial studies revealed the possibility of a catecholate ligand. The invention provides a family of such catecholate compounds that bind NGAL and chelate iron within the NGAL calyx with nanomolar and/or subnanomolar affinity.

### Compositions & Complexes

In preferred embodiments, the present invention provides compositions, such as pharmaceutical composition that comprise one or more of the compounds of the invention together with one or more lipocalins, or more preferably still, one or more compounds of the invention together with iron and one or more lipocalins. Combination pharmaceutical compositions and/or complexes that are contemplated by the present invention include, but are not limited to lipocalin:siderophore compositions that comprise Lipocalin:compound of Formula I, lipocalin:compound of Formula I(a), lipocalin:compound of Formula I(b), lipocalin:compound of Formula II, lipocalin:compound of Formula III, lipocalin:3-methylcatechol, lipocalin:4-methylcatechol, lipocalin:rosmarinic acid, lipocalin:myricetin, lipocalin:epigallocatechin gallate, lipocalin:pyrogallol, NGAL:2,3-dihydroxybenzoic acid, lipocalin:3,4-dihydroxybenzoic acid or lipocalin:ellagic acid. In preferred embodiments, the lipocalin component of the above listed lipocalin:siderophore compositions is NGAL. In further preferred embodiments, the above compositions also comprise iron, which is bound to the siderophore component of the lipocalin:siderophore composition. In some embodiments, the above recited components of the lipocalin:siderophore compositions are bound by covalent or non-covalent bonds, or both. As used herein the phrase “compositions of the invention” comprises all of the combination compositions/complexes described above, and all combination compositions/complexes comprising a catecholate compound of the invention and a lipocalin.

The compounds of the invention are siderophores and accordingly have an intrinsic ability to bind to iron. In some preferred embodiments the pharmaceutical compositions of the invention comprise a compound of the invention having iron bound thereto which is in turn bound to a lipocalin, such as NGAL. Such complexes can serve as iron donors by releasing their bound iron. In other embodiments, the compositions of the invention comprise a compound of the invention not having iron bound thereto which is bound to a lipocalin, such as NGAL. These complexes may serve as iron chelators. The pharmaceutical compositions of the invention may also comprise any of the above components in unbound form, and the components may bind either in the composition and/or in the body after administration of the compositions, for example upon exposure to favorable conditions, such as pH. The components of the compositions of the invention may also be provided individually in separation pharmaceutical compositions, wherein the components combine later, for example in the body, for example following co-administration of a pharmaceutical composition comprising a compound of the invention and a pharmaceutical composition comprising a lipocalin, and, optionally, a pharmaceutical composition comprising iron. Thus, in one embodiment each of the compound of the invention, the lipocalin, and optionally the iron components may be administered separately in separate pharmaceutical compositions, such as by co-administration or sequential administration. In another embodiment, the compound of the invention and iron are administered together in the same pharmaceutical composition (preferably in bound form—i.e. where the iron is bound to the compound) and the lipocalin is administered separately in a separate pharmaceutical compositions, such as by co-administration or sequential administration. In a preferred embodiment, all three components, i.e. the compound of the invention, iron (optionally), and the lipocalin are present in the same pharmaceutical composition and can thus be administered together by administering a single pharmaceutical composition. Preferably the pharmaceutical composition comprises the above components in bound form. For example, in preferred embodiments, if iron is present in the pharmaceutical composition, it is bound to the compound of the invention. Similarly, in preferred embodiments, the compound of the invention is preferably bound to the lipocalin in the pharmaceuctial composition. However, it is not essential that the components are bound to each other in the pharmaceutical composition—for example, they may bind upon exposure to favorable binding conditions, such as pH, for example following administration, or following admixture with another composition. Furthermore, the compositions, of the invention may comprise any of the above components in unbound form, and the components may bind either in the composition and/or in the body after administration of the compositions, for example upon exposure to favorable conditions, such as pH. Furthermore, compositions in which a proportion of the compound:lipocalin complexes have iron bound thereto may serve as either iron donors or iron chelators, depending on factors such as the amount of free iron, the pH, the location of the complex, and the like.

The binding of a compound of the invention (with or without associated iron) to a lipocalin can be measured using any suitable technique, such as those described in Goetz et al. (2002), the contents of which are hereby incorporated by reference, by using a surface plasmon resonance assay, or by any other suitable assay.

The lipocalin NGAL has a conserved structure which comprises a broad, shallow calyx lined with polar and positively charged residues, as described by Coles et al. 1999, Goetz et al., 2000, Goetz et al 2002, and Holmes et al. 2005, the contents of which are hereby incorporated by reference. In preferred embodiments, the compounds of the invention bind to lipocalins such as NGAL via electrostatic and/or cation-π interactions, as described in Goetz et al. 2002 and Holmes et al. 2005, the contents of which are hereby incorporated by reference. In further preferred embodiments, the compounds of the invention bind to to “pocket 1” of the trolobate calyx of NGAL, as described by Goetz et al., 2002 and Holmes et al. 2005. In further preferred embodiments, the compounds of the invention interact with NGAL via the side chains of the two lysine residues of NGAL (amino acid residues K125 and K134 of SEQ ID NO. 1 and 2, or corresponding residues) that are located in the area of “pocket 1” of the trolobate calyx of NGAL, as described in Goetz et al., (2002) and Holmes et al. (2005), the contents of which are hereby incorporated by reference. In further preferred embodiments, in the presence of iron, the compounds of the invention bind to a lipocalin, such as NGAL, with nanomolar or subnanomolar affinity. In one embodiment, in the presence of iron, the compounds of the invention bind to a lipocalin, such as NGAL, with an affinity in the range of about 0.01 to about 100 nanomolar, or in the range of about 0.1 nanomolar to about 10 nanomolar, or in the range of about 0.5 nanomolar to about 5 nanomolar.

### Crystals

In certain embodiments, the present invention provides crystals (co-crystals) comprising a compound of the invention, optionally bound to iron, and NGAL. For example, the present invention provides a crystal comprising catechol, iron, and NGAL, a crystal comprising 4-methylcatechol, iron, and NGAL, a crystal comprising 3-methylcatechol, iron, and NGAL, a crystal comprising pyrogallol, iron, and NGAL, a crystal comprising caffeic acid, iron, and NGAL, and a crystal comprising rosmarinic acid, iron, and NGAL. In preferred embodiments, the crystals comprise NGAL having the amino acid sequence of SEQ ID NO. 1 or 2. Specific examples of such crystals are provided in the Examples section of this application. In each of such crystals, the NGAL protein has the amino acid sequence of SEQ ID 2 (Human NGAL C87S) which was expressed and purified as previously described in Goetz et al. (2000), Goetz et al. (2002), and Holmes et al., (2005), the contents of which are hereby incorporated by reference.

Thus, in one embodiment, the present invention provides a crystal comprising NGAL in association with Fe-catechol, wherein the NGAL protein has the amino acid sequence of SEQ ID NO. 2, and wherein said crystal forms in space group P4₁2₁2 with unit cell dimensions a=b=115.4 and c=188.8. Further parameters of such a crystal are provided in the Examples.

In another embodiment, the present invention provides a crystal comprising NGAL in association with Fe-4-methyl-catechol, wherein the NGAL protein has the amino acid sequence of SEQ ID NO. 2, and wherein said crystal forms in space group P4₁2₁2 with unit cell dimensions a=b=114.9 and c=119.6. Further parameters of such a crystal are provided in the Examples.

In another embodiment, the present invention provides a crystal comprising NGAL in association with Fe-3-methyl-catechol, wherein the NGAL protein has the amino acid sequence of SEQ ID NO. 2, and wherein said crystal forms in space group P4₁2₁2 with unit cell dimensions a=b=115.1 and c=118.6. Further parameters of such a crystal are provided in the Examples.

In another embodiment, the present invention provides a crystal comprising NGAL in association with Fe-pyrogallol, wherein the NGAL protein has the amino acid sequence of SEQ ID NO. 2, and wherein said crystal forms in space group P4₁2₁2 with unit cell dimensions a=b=116.3 and c=120.8. Further parameters of such a crystal are provided in the Examples.

In another embodiment, the present invention provides a crystal comprising NGAL in association with Fe-caffeic acid, wherein the NGAL protein has the amino acid sequence of SEQ ID NO. 2, and wherein said crystal forms in space group P4₁2₁2 with unit cell dimensions a=b=114.42 and c=119.15. Further parameters of such a crystal are provided in the Examples.

In another embodiment, the present invention provides a crystal comprising NGAL in association with Fe-rosmarinic acid, wherein the NGAL protein has the amino acid sequence of SEQ ID NO. 2, and wherein said crystal forms in space group P4₁2₁2 with unit cell dimensions a=b=114.81 and c=118.70. Further parameters of such a crystal are provided in the Examples.

The present invention also provides methods of use of such crystals, for example in studying and/or modellling the interaction of NGAL with catecholate compounds and/or iron and for rational design of drugs that affect the interaction of NGAL with catecholate compounds and/or iron.

### Methods of Treatment and Other Uses

The present invention is based, in part, on the discovery of a family of catechol-related iron-binding compounds that bind with high affinity to lipocalin proteins, such as neutrophil gelatinase-associated lipocalin (“NGAL”), and the discovery that complexes comprising these catecholate compounds and a lipocalin are able to bind to, transport, and release iron in vivo. Thus, the catechol-related compounds of the invention, and combination compositions/complexes containing such catechol-related compounds and a lipocalin, may be used as iron chelators and/or iron donors and may be useful in the treatment of various conditions, diseases and disorders associated with excessive iron levels and/or iron deficiency.

In one embodiment, the compositions of the invention, may be used to treat any condition, disease or disorder associated with excessive iron levels or iron overload and/or iron deficiency, including each of the conditions diseases and disorders described herein.

In other embodiments, other siderophore:lipocalin and/or siderophore:lipocalin:iron complexes, such as lipocalin:enterochelin and NGAL:enterochelin complexes, may be used in conjuction with the methods of treatment described herein, which include methods of delivering iron, e.g. to treat conditions associated with iron deficiency. Any of the lipocalins and siderophores described herein may be used in such methods.

Large amounts of free iron in the bloodstream can lead to cell damage, especially in the liver, heart and endocrine glands. The causes of excess iron may be genetic, for example the iron excess may be caused by a genetic condition such as hemochromatosis type 1 (classical hemochromatosis), hemochromatosis type 2A or 2B (juvenile hemochromatosis), hemochromatosis type 3, hemochromatosis type 4 (African iron overload), neonatal hemochromatosis, aceruloplasminemia, or congenital atransferrinemia. Examples of non-genetic causes of iron excess include dietary iron overload, transfusional iron overload (due to a blood transfusion given to patients with thalassaemia or other congenital hematological disorders), hemodialysis, chronic liver disease (such as hepatitis C, cirrhosis, non-alcoholic steatohepatitis), porphyria cutanea tarda, post-portacaval shunting, dysmetabolic overload syndrome, iron tablet overdose (such as that caused by consumption by children of iron tablets intended for adults), or any other cause of acute or chronic iron overload.

The two common iron-chelating agents available for the treatment of iron overload are deferoxamine (DFO) and deferiprone (oral DFO). Due to its high cost and need for parenteral administration, the standard iron chelator deferoxamine is not used in many individuals with acute and/or chronic iron poisoning. Deferoxamine must be administered parenterally, usually as a continuous subcutaneous infusion over a 12-hour period, from three to seven times a week. Treatment is time consuming and can be painful. As a result compliance is often poor. Side-effects include local skin reactions, hearing loss, nephrotoxicity, pulmonary toxicity, growth retardation and infection Deferiprone is the only orally active iron-chelating drug to be used therapeutically in conditions of transfusional iron overload. It is indicated as a second-line treatment in patients with thalassaemia major, for whom deferoxamine therapy is contraindicated, or in patients with serious toxicity to deferoxamine therapy. Deferiprone is an oral iron-chelating agent which removes iron from the heart, the target organ of iron toxicity and mortality in iron-loaded thalassaemia patients. However, although deferiprone offers the advantage of oral administration, it is associated with significant toxicity and there are questions about its long-term safety and efficacy. It is recommended to be used in patients who are unable to use desferrioxamine because of adverse effects, allergy, or lack of effectiveness. Deferiprone is associated with serious safety issues include genotoxicity, neutropenia and agranulocytosis. Weekly monitoring of neutrophils is recommended. Gastrointestinal and joint problems can occur and liver toxicity has been reported. Therefore, there is clearly a need for alternative convenient, safe, and effective iron chelation therapies, such as those provided by the present invention.

Iron deficiency is the most common nutritional deficiency in humans. As an iron delivery agent, the complexes provided by the invention may be used as therapy for iron deficiency anemia, for example, for patients treated with chronic hemodialysis and/or peritoneal dialysis, such as those with End Stage Renal Disease (ESRD). The complexes provided by the invention may also be used as, or as part of, an iron supplementation regimen, for example in the treatment of anemia. Chronic kidney disease (CKD) patients are generally provided with limited access to anemia therapy until immediately prior to the initiation of dialysis, although multiple studies and medical organizations have pointed to the importance of early treatment of anemia in improving CKD outcomes. With improved safety and bioavailability, the complexes provided by the invention may be used to provide better and earlier care. Other non-limiting examples of diseases associated with iron deficiency/anemia include cancer, HIV/AIDS, hepatitis, autoimmune diseases, and cardiovascular disease.

The invention provides a novel reversible mechanism of iron chelation/iron delivery to cells. The compounds and complexes provided by the invention, represent potential therapeutic agents for delivering soluble bioavailable iron that does not undergo cell damaging redox chemical reactions.

Non-limiting examples of causes of iron deficiency include loss of iron due to loss of blood, chronic bleeding (for example from gastrointestinal disease, laryngological bleeding, bleeding from the respiratory tract, or bleeding of the gastric mucosa caused by anti-inflammatory drugs), inadequate iron intake, pregnancy or any other condition that increases the body's demand for iron, substances (e.g., in diet or drugs) that interfere with iron absorption, nutritional deficiency (e.g., due to failure to eat iron-containing foods, or eating a diet heavy in food that reduces the absorption of iron, or both), malabsorption syndromes, inability to absorb iron because of damage to or loss of the intestinal lining surface area (e.g., surgery involving the duodenum, Crohn's disease, or celiac sprue), fever to control bacterial infection, hemosiderinuria, pulmonary siderosis, or inflammation leading tohepcidin-induced restriction on iron release from enterocytes.

The compounds and compositions/complexes described herein may be used to chelate free iron and clear the excess iron from the body via the kidneys, for example to reduce toxic circulating levels of iron to below toxic levels. The compounds and compositions/complexes described herein may be used to deliver and/or donate iron. The invention also provides methods, pharmaceuctical formulations, kits, and medical devices that comprise the compounds and/or compositions/complexes described herein and which may be useful to treat an iron overload disorder and/or clear excess iron and/or to deliver/donate iron. Pharmaceutical formulations include those suitable for oral or parenteral (including intramuscular, subcutaneous and intravenous) administration. Examples of medical devices provided by the invention include, but are not limited to, beads, filters, shunts, stents, and extracorporeal loops which are coated with or otherwise contain a compound or composition/complex as described herein, such that the device is implanted in or otherwise administered to a subject in a manner which permits the composition/complex to chelate or absorb excess iron in the subject and/or to deliver/donate iron.

In one embodiment, the compounds, compositions and complexes described herein can be used to reduce a toxic amount of iron present in the kidneys upon administration to a subject in need thereof. Modes of administration are described herein and known in the art, including intravenous administration. Disruption of iron transport through or mislocalization of iron in the kidney may contribute to oxidative injury in the proximal tubule, for example the onset of acute tubular necrosis (ATN). ATN is a common cause of renal failure in hospitalized patients. ATN can be caused by ischemia of the kidneys (lack of oxygen to the tissues), or by exposure to materials, such as medications, that are toxic to the kidney (nephrotoxic agents). The compounds, compositions and complexes described herein may be useful for the treatment of such kident conditions.

As described in the Examples, lipocalin:catecholate:iron complexes can form in vivo, traffic iron in the blood and then clear this iron at the luminal face of the kidney's proximal tubule. In addition, these complexes may chelate iron in a form that abolishes its involvement in the undesirable Fenton reaction, thus making the bound iron redox inactive. As described in the Example, the pH sensitivity of the lipocalin:catecholate:iron complexes of the invention correlate with endosomal pH. Below pH 6.5 the complexes of the invention are typically dissociated, and thus release iron. This dissociation may be reversible allow re-binding. This pH dependent mechanism allows the compositions/complexes of the invention not only to traffic iron in the blood, but also to recycle it to cells. Based on these findings, the invention provides high affinity iron trafficking compositions/complexes that form when iron, a compound of the invention, and a lipocalin, are present together. The invention further provides that the ability of the compounds, compositions and complexes of the invention to function as iron donors or iron chelators depends on the pH of the environment surrounding the compounds compositions and complexes.

Administration of a therapeutically effective amount of the compounds and compositions of the invention can be accomplished via any mode of administration suitable for therapeutic agents. One of skill in the art can readily select mode of of administration without undue experimentation. Suitable modes may include systemic or local administration such as oral, nasal, parenteral, transdermal, subcutaneous, vaginal, buccal, rectal, topical, intravenous (both bolus and infusion), intraperitoneal, or intramuscular administration modes. In preferred embodiments, oral of intravenous administration is used. In other preferred embodiments, the compositions of the invention are administered directly to the desired site of action, such as for example, the kidney, for example by local injection or local infusion or by use of (e.g. conjugation to) agents useful for targeting proteins or pharmaceuticals to specific tissues, such as antibodies etc.

Depending on the intended mode of administration, the compounds and compositions of the invention, in a therapeutically effective amount, may be in solid, semi-solid or liquid dosage form, such as, for example, injectables, tablets, suppositories, pills, time-release capsules, elixirs, tinctures, emulsions, syrups, powders, liquids, suspensions, or the like. In one embodiment the compounds and compositions of the invention may be formulated in unit dosageb forms, consistent with conventional pharmaceutical practices. Liquid, particularly injectable, compositions can, for example, be prepared by dissolution or dispersion. For example, a compound or composition of the invention can be admixed with a pharmaceutically acceptable solvent such as, for example, water, saline, aqueous dextrose, glycerol, ethanol, and the like, to thereby form an injectable isotonic solution or suspension.

Parental injectable administration can be used for subcutaneous, intramuscular or intravenous injections and infusions. Injectables can be prepared in conventional forms, either as liquid solutions or suspensions or solid forms suitable for dissolving in liquid prior to injection. One embodiment, for parenteral administration, employs the implantation of a slow-release or sustained-released system, according to U.S. Pat. No. 3,710,795, incorporated herein by reference.

The compounds and compositions of the invention can be sterilized and may contain any suitable adjuvants, preservatives, stabilizers, wetting agents, emulsifying agents, solution promoters, salts (e.g. for regulating the osmotic pressure), pH buffering agents, and/or other pharmaceutically acceptable substances, including, but not limited to, sodium acetate or triethanolamine oleate. In addition, the compositions of the invention may also contain other therapeutically useful substances, such as, for example, other iron chelators or iron donors or other agents useful in the treatment of of iron deficieny or iron overload, or other agents useful in the treatment of any of the conditions described herein.

Compositions can be prepared according to conventional mixing, granulating or coating methods, respectively, and the present pharmaceutical compositions can contain from about 0.1% to about 99%, preferably from about 1% to about 70% of the compound or composition of the invention by weight or volume.

The dose and dosage regimen to be used can be determine in accordance with a variety of factors including the species, age, weight, sex and medical condition of the subject; the severity of the condition; the route of administration; the renal or hepatic function of the subject; and the particular compound or composition employed. A person skilled in the art can readily determine and/or prescribe an effective amount of a compound or composition of the invention useful for treating or preventing a condition, for example, taking into account the factors described above. Dosage strategies are also provided in L. S. Goodman, et al., The Pharmacological Basis of Therapeutics, 201-26 (5th ed.1975), which is herein incorporated by reference in its entirety. In one embodiment, compositions of the invention are administered such that the lipocalin component is administered at a dose range of about 1 to about 100 mg/kg body weight, and typically at a dosage of about 1 to about 10 mg/kg body weight or is administered at a dose that results in a concentration in the range of about 0.1 ng/ml to about 100 ng/ml, e.g., in the range of about 1.0 ng/ml to about 20 ng/ml, in the blood. The amount of the siderophore compounds will be chosen accordingly, such that the desired stoichiometry, e.g. 1:1 binding with NGAL is achieved.

In addition to the above methods of treatment, the compounds and compositions of the invention may be useful to chelate and/or remove iron from samples, wherein the sample are not in a subject's body. Thus, in one embodiment, the present invention provides a method for removing iron from a fluid, the method comprising admixing the fluid with a composition a compound of the invention and a lipocalin, such as NGAL, wherein the composition or compound either does not contain iron or contains a small amount of iron (i.e. an amount such that the composition or compound is not saturated with iron and can chelate more iron from the sample), for a period of time sufficient for iron in the sample to bind to the by the compound or composition. In one embodiment, the compound or composition having iron bound thereto may then be removed the composition from the sample.

In preferred embodiments, the sample is a biological fluid, such as blood, serum, plasma, or urine. In certain embodiments the compounds or compositions of the invention are admixed with the sample outside the body, e.g. in an extracorporeal device, and the sample is then delivered to or returned to the body of a subject after chelation and/or removal of the iron. For example, such methods can be used to chelate and/or remove excess iron in blood samples for transfusion, or in a dialysis procedure. For example, blood or another bodily fluid from a subject may be removed from the body, treated with a compound or composition of the invention to chelate or remove excess iron, and then returned to the subject.

### Diagnostic and Detection Methods

The present invention also provides methods for detecting the presence of a lipocalin, such as NGAL in a sample. In one embodiment, such methods comprise (a) contacting with iron a compound capable of binding the lipocalin (e.g. NGAL) and iron (e.g. a compound of the invention), thereby forming a complex between the compound and the iron; (b) contacting the sample with the complex of step (a); and (c) determining the presence of the lipocalin in the sample of step (b) as compared to a sample that does not contain the lipocalin.

In one embodiment, the sample is a biological sample. In a preferred embodiment, the biological sample is a bodily fluid, such as urine, saliva, a vaginal secretion, or blood.

In one aspect of the methods provided by the invention, the compound used in step (a) comprises a bacterial siderophore. In another aspect, the compound comprises a mammalian siderophore. In yet another aspect, the compound comprises a compound of the invention. In one embodiment of the methods of the invention, the compound is conjugated to a detectable label. In another embodiment, the detectable label is a chromophore or a fluorophore.

In one embodiment of the invention, the “determining” performed in step (c) comprises measuring the pH stability of the complex of step (a). In another embodiment, the determining comprises measuring the redox stability of the complex of step (a). In a further embodiment, the determining comprises measuring absorbance of a chromophore or a fluorophore.

In preferred embodiments of the invention, the lipocalin to be detected is NGAL.

Other methods useful for the detection of a lipocalin, such as NGAL, in a sample are also within the scope of the present invention. Such methods are are based on the use of siderophores to detect a lipocalin, such as NGAL, in a sample. In one embodiment, a siderophore is used to capture or bind a urinary lipocalin, such as NGAL. In one embodiment, the siderophore is conjugated, immobilized, or both. The siderophore may be conjugated to a protein, such as bovine serum albumin (BSA): gold; or an affinity matrix, such as a bead or resin. The detection method may be carried out in any format where the direct or indirect contact of the lipocalin, such as NGAL, with the siderophore results in a signal that is detectable or quantifiable. The method can be carried out to diagnose a disease or disorder, such as acute kidney injury. One embodiment of a diagnostic method is described in Example 9.

In another embodiment, the present invention provides a method for determining whether a subject is suffering from a condition, or may be at risk for a conditions, the method comprising: (a) obtaining a sample of body fluid from a subject; (b) contacting an amount of the sample with a siderophore, such as one of the catecholate compounds of the invention, in order to allow binding of a lipocalin (such as NGAL) in the sample to the siderophore; (c) determining whether the amount of lipocalin bound is above or below an amount of lipocalin bound by fluid from a subject without condition, wherein a greater or lesser amount of lipocalin bound from the sample indicates that the subject is suffering from a condition or may be at risk for a condition.

Such methods may be useful for determining whether a subject is suffering from or at risk for any condition that may be associated with altered levels or activity of a lipocalin, such as bladder cancer, a kidney disease, a urinary track disease or disorder, a brain disease or disorder, a liver disease, kidney failure, a kidney cancer, diabetes, a viral infection, a brain cancer, and/or a bacterial infection.

In another embodiment, the present invention provides a method for detecting a lipocalin, such as NGAL, in urine, the method comprising:(a) obtaining or generating a chromatographic stationary phase, wherein the stationary phase comprises: (1) a capture line comprising NGAL immobilized on the stationary phase; or (2) a capture line comprising an antibody or fragment thereof that binds NGAL immobilized on the stationary phase; or (3) a first capture line comprising NGAL immobilized on the stationary phase and a second capture line comprising an antibody or fragment thereof that binds NGAL immobilized on the stationary phase; and (4) a conjugate matrix comprising a siderophore conjugated to gold, wherein the conjugate matrix is attached to a surface of the stationary phase; and (b) applying a mobile phase to the stationary phase, wherein the mobile phase comprises urine; and (c) determining the presence of the lipocalin, such as NGAL, in the mobile phase by detecting a detectable signal. In preferred embodiments, the stationary phase comprises nitrocellulose paper. In other preferred embodiments, the conjugate matrix comprises glass fiber. In other preferred embodiments, the siderophore comprises TRENCAM, MECAM, a myo-inositol-derived enterobactin, or a compound of the invention. In further preferred embodiments, the siderophore is further conjugated to bovine serum albumin.

In preferred embodiments of all of the diagnostic/detection methods described herein, the sample used is a biological sample, such as urine, saliva, a vaginal secretion, or blood. In preferred embodiments, the biological sample is urine.

The following Examples further illustrate certain embodiments of the present invention. These Examples are set forth to aid in the understanding of the invention, and should not be construed to limit in any way the scope of the invention as defined herein.

### EXAMPLES

### Example 1

### Identification of the NGAL:Catechol:Iron Complex

As described in the below Examples, mouse and human urine samples were screened to identify “endogenous” NGAL ligands capable of binding iron (i.e., ligands having siderophore activity). Catechol and related molecules (collectively referred to herein as “catechols”) are metabolites of amino acids and plant polyphenols.

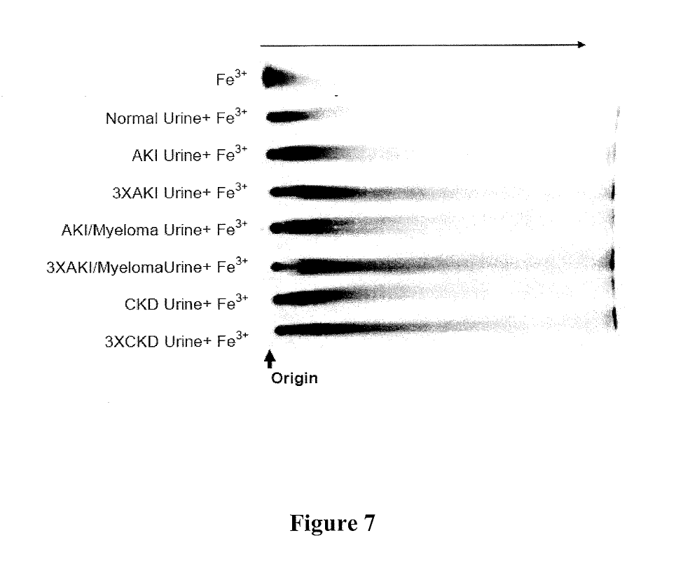

Using paper chromatography, protein-free filtrates (<3 KDa) of urine were found to be able to solubilize iron, implying the presence of low molecular weight iron chelating molecules (FIG. 7). It was then found that these filtrates could retain iron when mixed with empty, ligand-free NGAL (Mori et al., 2005) even after the protein-urine complex was washed repetitively on a 10 KDa cutoff filter or rapidly by gel filtration, implying that the urine compounds had bound the protein as well as iron. Indeed, after induction of NGAL with Lipopolysaccharide in vivo, low molecular weight urine filtrates (<3 KDa) from wild type mice had lower iron retaining activity than urine filtrates from NGAL deleted littermates, implying that urinary iron chelators had been sequestered from the urine by the induced NGAL protein.

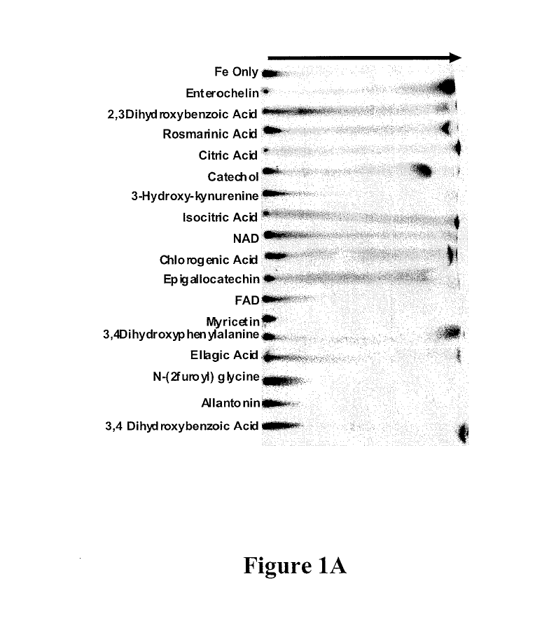

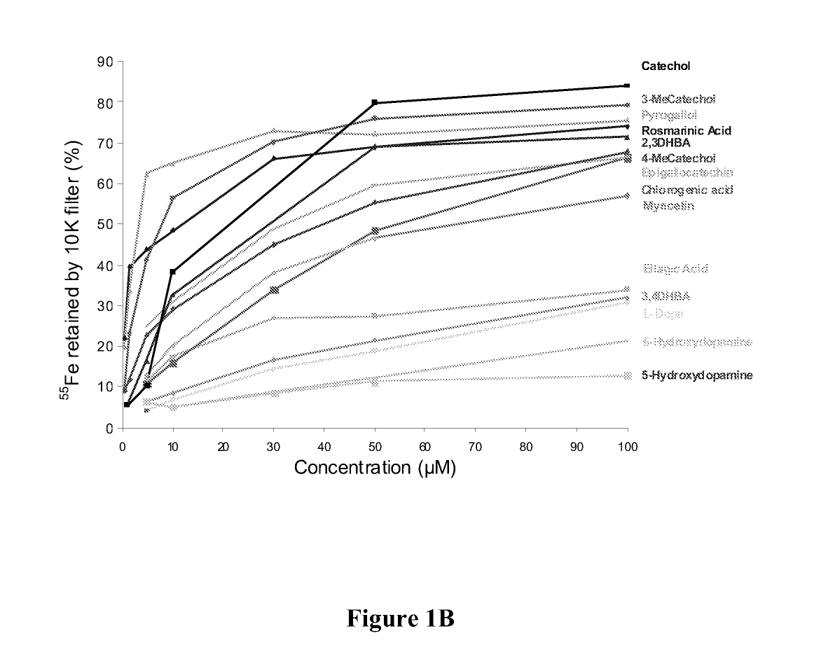

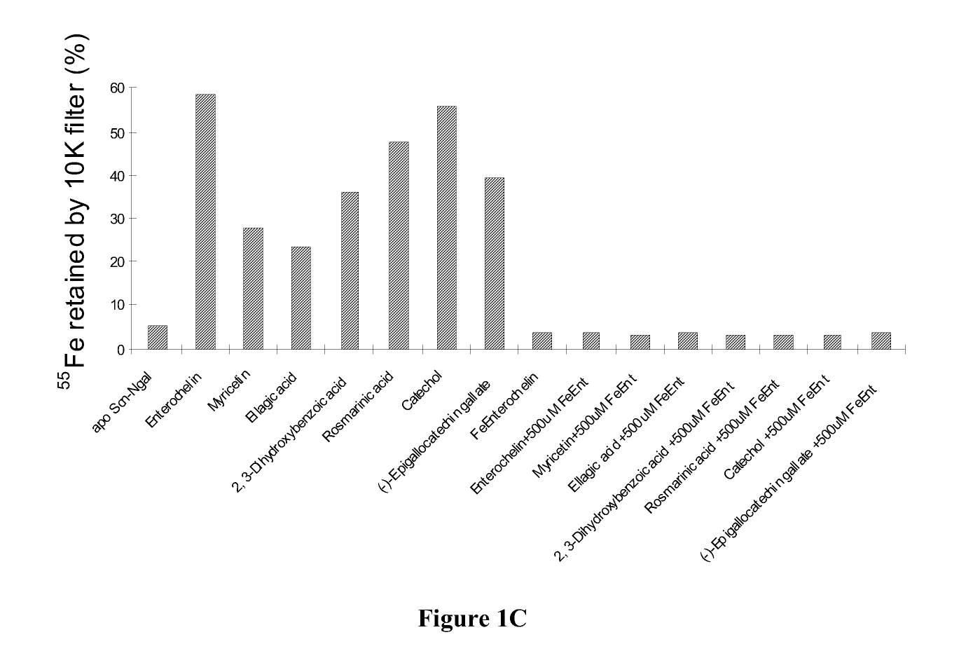

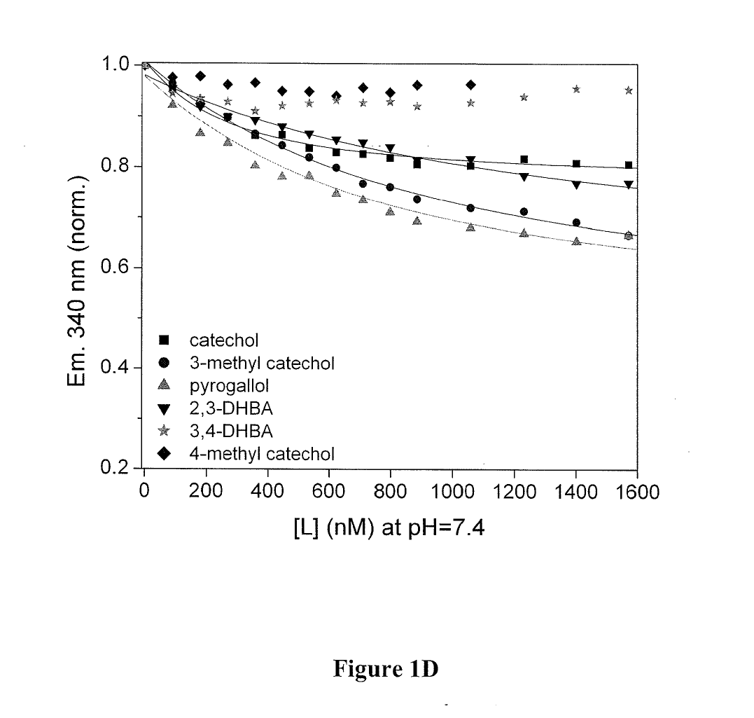

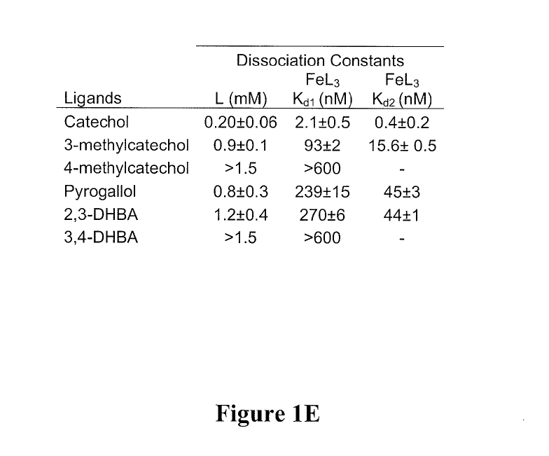

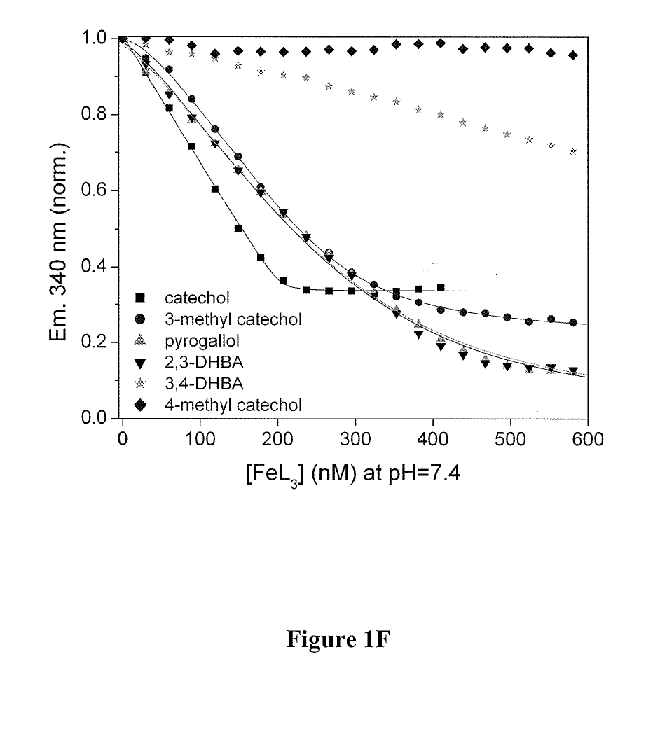

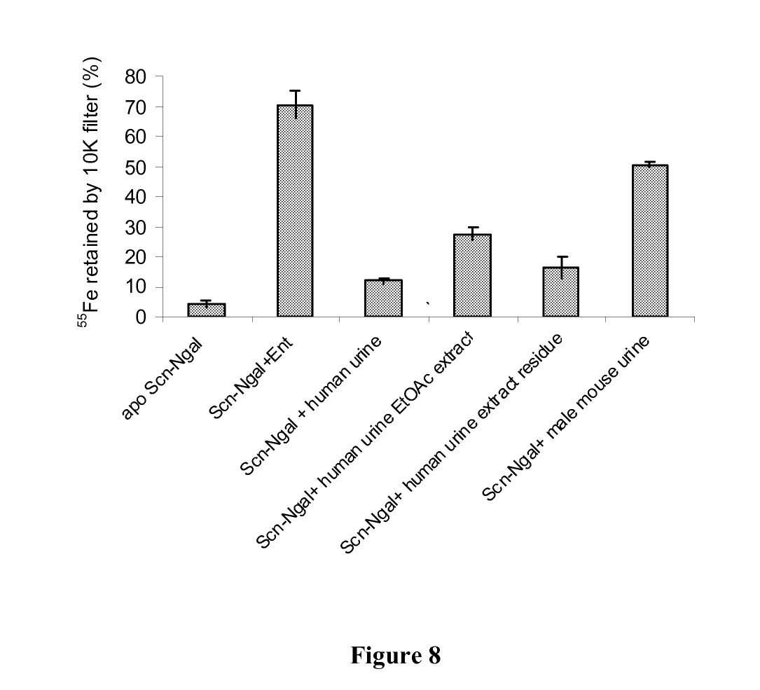

The activity found in urine filtrates (<3 KDa) was partially extractable with ethylacetate, demonstrating that it included organic molecules (FIG. 8). Subsequently, a screen of urinary organic compounds (Wishart et al., (2007). HMDB: the Human Metabolome Database. Nucleic Acids Res. 35, D521-526) identified 18 that solubilized iron (some of which are shown in FIG. 1A; Supplemental Table 1), nine of which resulted in iron retention by NGAL (Table A; FIG. 1B, C).

Table A below shows the structures of nine catechols that were found to bind NGAL and chelate iron within the NGAL calyx with very high affinity (e.g., subnanomolar affinity). Table B shows the structures of all of the compounds identified in urine that bind to NGAL.

TABLE A
High-affinity NGAL binding catechols

| Compound | Structure |
|---|---|
| Catechol | 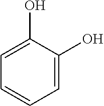 |
| 3-methylcatechol | 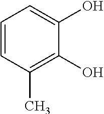 |
| 4-methylcatechol | 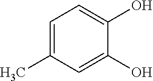 |
| Rosmarinic acid | 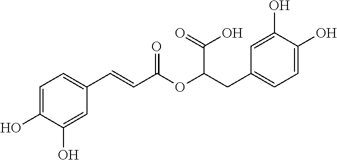 |
| Myricetin | 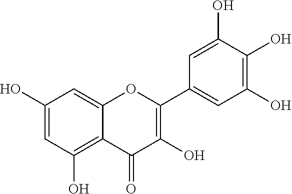 |
| (−) Epigallo- catechin gallate | 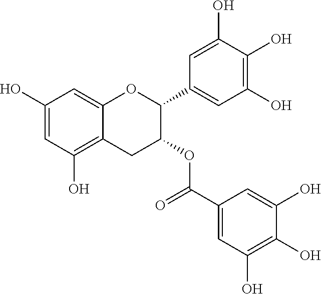 |
| Benzene 1,2,3 Trial (Pyrogallol) | 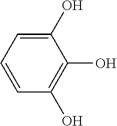 |
| 2,3-Dihydroxy- benzoic acid | 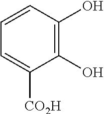 |
| Ellagic acid | 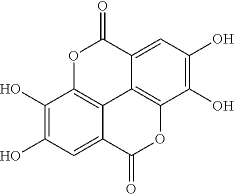 |

Note that all of the compounds described in Table A above are commercially available, for example from Sigma Aldrich.

Among these compounds, catechol, 3-methylcatechol, 4-methylcatechol and pyrogallol (3-hydroxycatechol) demonstrated the highest activities and even compounds with more limited activity were related catecholate type molecules. Hence, an unbiased screen of urinary compounds revealed that a group of active molecules all contained the catechol functional group. The interaction of these compounds with iron was specific in that iron binding activity was lost upon O-methylation or O-sulfonation of the catechol hydroxyls (Table B), consistent with the dihydroxy moieties providing essential binding sites.

TABLE B
Urinary Compounds

| | | Fe: | NGAL: Chelator: |
|---|---|---|---|
| Compound | Structure | Chromatography | Fe % Retention |
| Catechol | 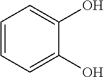 | Yes | 56 |
| Guaiacol | 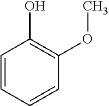 | No | 4 |
| 1,2- Dimethoxybenzene | 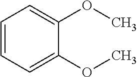 | No | 3 |
| Catechol cyclic sulfonate | 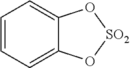 | No | 6 |
| Catechol sulfonate sodium | 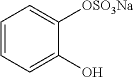 | No | 4 |
| 3-methylcatechol | 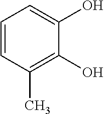 | Yes | 56 |
| 4-methylcatechol | 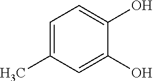 | Yes | 29 |
| 3,4-Dihydroxy-DL- phenylalanine | 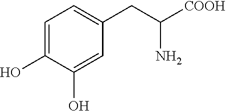 | Yes | 12 |
| Dihydroxyphenyl alanine (L-DOPA) | 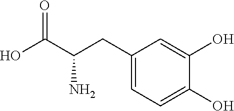 | Yes | 10 |
| DL- Norepinephrine•HCl | 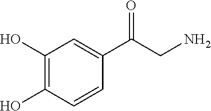 | No | 5 |
| Caffeic acid | 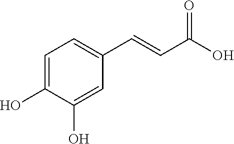 | No | 7 |
| Ferulic acid | 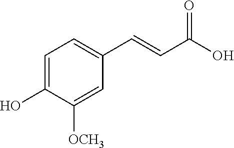 | No | 3 |
| Caffeic acid phenethyl ester | 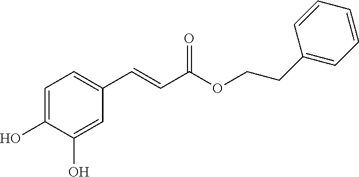 | No | 8 |
| Rosmarinic acid | 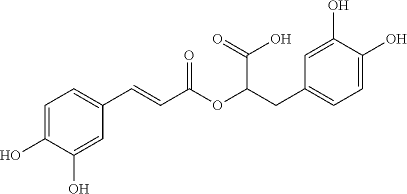 | Yes | 50 |
| Chlorogenic acid | 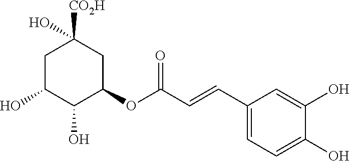 | Yes | 6 |
| 5- Hydroxydopamine | 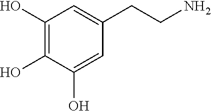 | No | 10 |
| 6- Hydroxydopamine | 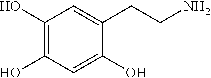 | No | 4 |
| Myricetin | 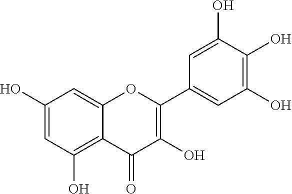 | No | 28 |
| (−) Epigallocatechin gallate | 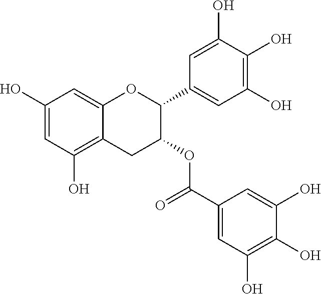 | Yes | 45 |
| Benzene 1,2,3 Trial (Pyrogallol) | 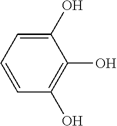 | Yes | 73 |
| 2,3- Dihydroxybenzoic acid | 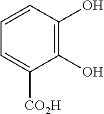 | Yes | 35 |
| 2,3 Dimethoxybenzoic acid | 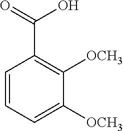 | No | 6 |
| 3- Hydroxyanthranilic acid | 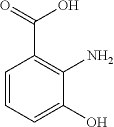 | No | 10 |
| 3,4- Dihydroxybenzoic Acid | 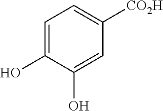 | Yes | 5 |
| Salicylic acid | 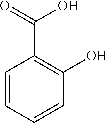 | No | 15 |
| Ellagic acid | 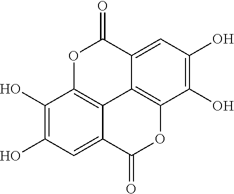 | Yes | 24 |
| Homogentisic acid | 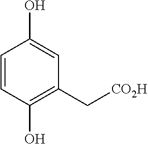 | No | 7 |
| Gentistic Acid | 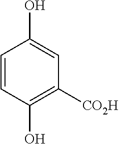 | No | 6 |
| 3-Hydroxy-DL- kynurenine | 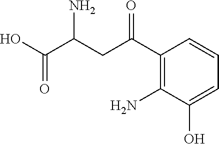 | Yes | 3 |
| L-Phenylalanine | 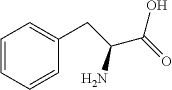 | No | 8 |
| N-Acetyl-DL-Â- phenylalanine | 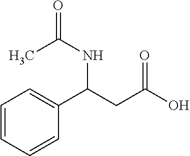 | No | 5 |
| L-Tryptophan | 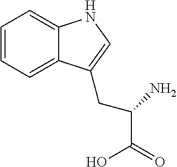 | No | 5 |
| 5- Hydroxytryptophan | 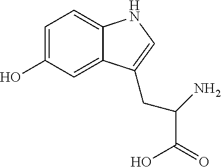 | No | 4 |
| 5-Hydroxy- indoleacetic acid | 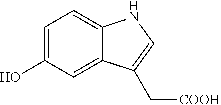 | No | 7 |
| Uracil | 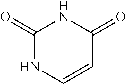 | No | 7 |
| Orotic acid | 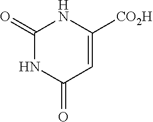 | No | 13 |
| DL- Dihdroorotic acid | 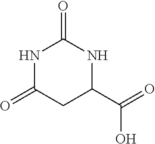 | No | 7 |
| Nicotinic acid | 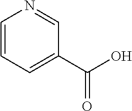 | No | 3 |
| 2,3- Pyridine- dicarboxylic acid | 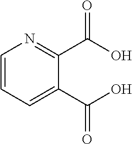 | No | 6 |
| Pyridoxal | 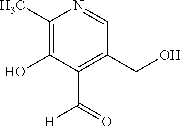 | No | 1 |
| 4-Pyridoxic Acid | 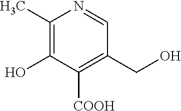 | No | 12 |
| 2-Furoylglycine | 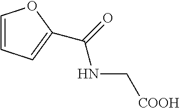 | No | 6 |
| Porphobilinogen | 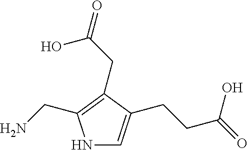 | No | 3 |
| Glycyl-L-proline | 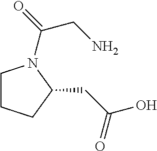 | No | 4 |
| Allantoin | 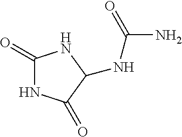 | No | 7 |
| Bilirubin | 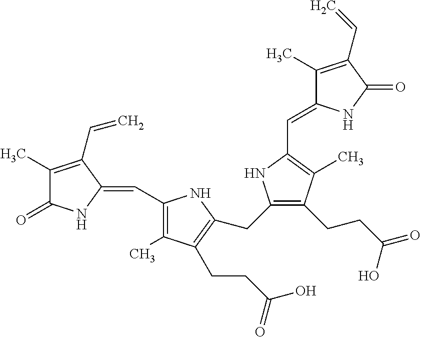 | No | 3 |
| Biliverdin HCl | 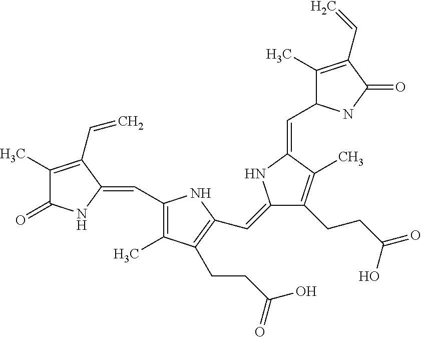 | No | 9 |
| Urobilin HCl | 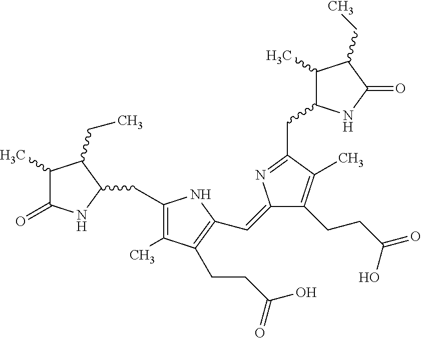 | No | 3 |
| Protoporphyrin IX | 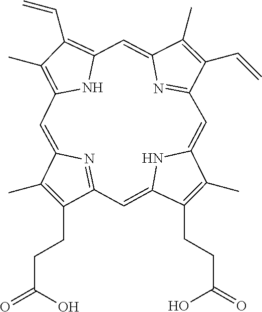 | No | 3 |
| FAD | 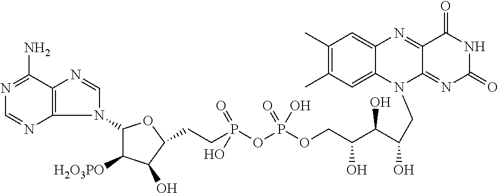 | Yes | 6 |
| NADP | 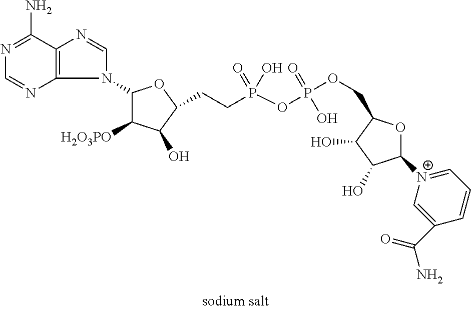 | Yes | 7 |
| NADPH | 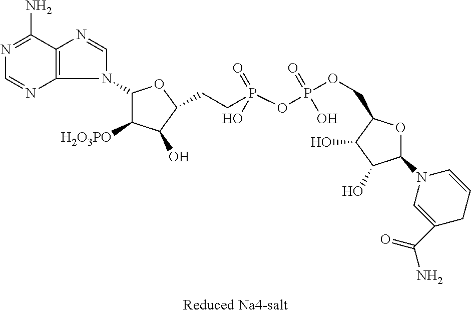 | Yes | 3 |
| NAD | 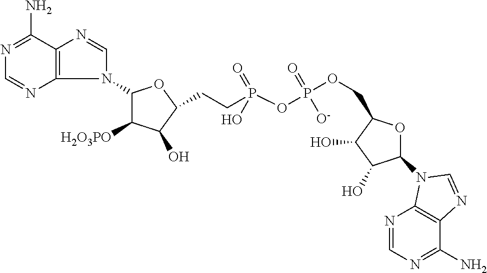 | No | 12 |
| Folic acid | 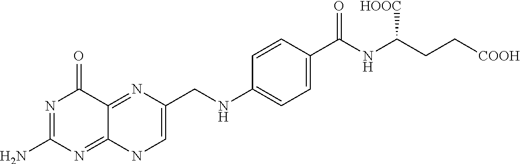 | No | 3 |
| Maleic acid | 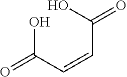 | No | 4 |
| Citric acid sodium | 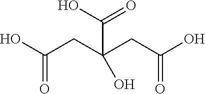 | Yes | 6 |
| Succinic acid | 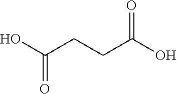 | No | 11 |
| 5-Aminolevulinic acid | 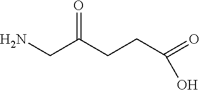 | No | 3 |
| Cis-Aconitic acid | 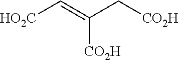 | No | 11 |
| Isocitric acid | 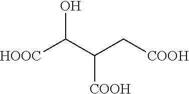 | Yes | 6 |

These findings are reminiscent of the interaction of NGAL with enterobacterial siderophores which, while structurally diverse, also contained catechol:iron units. In this light, the endogenous ligands mimicked the microbial siderophores by utilizing similar functional groups. NGAL recognizes the catechol groups of the bacterial siderophore enterochelin (or “Ent”) by interacting with the aromatic electron density of the catechols using cationic amino acids within the NGAL calyx, forming the so called cation-π bond (Hoette, T. M., Abergel, R. J., Xu, J.; Strong, R. K., Raymond, K. N. (2008). “The role of electrostatics in siderophore recognition by the immunoprotein Siderocalin”. J. Am. Chem. Soc. 130, 17584-17592). To determine whether NGAL recognized endogenous catechols by a similar type of interaction, the component of binding attributable to the cation-π bond was calculated (as described by Goetz et al., (2002), and Holmes et al., (2005), the contents of which are hereby incorporated by reference). The quadrupole moment of each bidentate ligand, as well as that of the corresponding quinone oxidation product was first established (Table B). Then the cation (Na⁺)-binding ability of the aromatic unit was calculated. These data indicated that the catechols (catechol>3-methylcatechol>pyrogallol) should have high affinity for NGAL based on optimized cation-π interactions when bound in the calyx.

TABLE C
Binding Energies
Theoretical quadrupole moments (Θ_{ZZ}, optimization and
calculation at the RHF/6-311G** level of theory) and cation
binding energies (p-Na⁺, calculated at the MP2/6-311++G**
and corrected for BSSE) for aromatic units studied.

| | Θzz | π-Na⁺ BE (kcal/mol) |
|---|---|---|
| Catechol | −9.643 | −30.88618469 |
| 3-methyl catechol | −10.220 | −21.21278474 |
| 4-methyl catechol | −9.690 | −21.04280054 |
| Pyrogallol | −9.951 | −19.63531703 |
| 2,3-DHBA | −6.463 | −16.18369249 |
| 3,4-DHBA | −8.435 | −15.92352296 |
| Quinone | 0.591 | −1.054721068 |

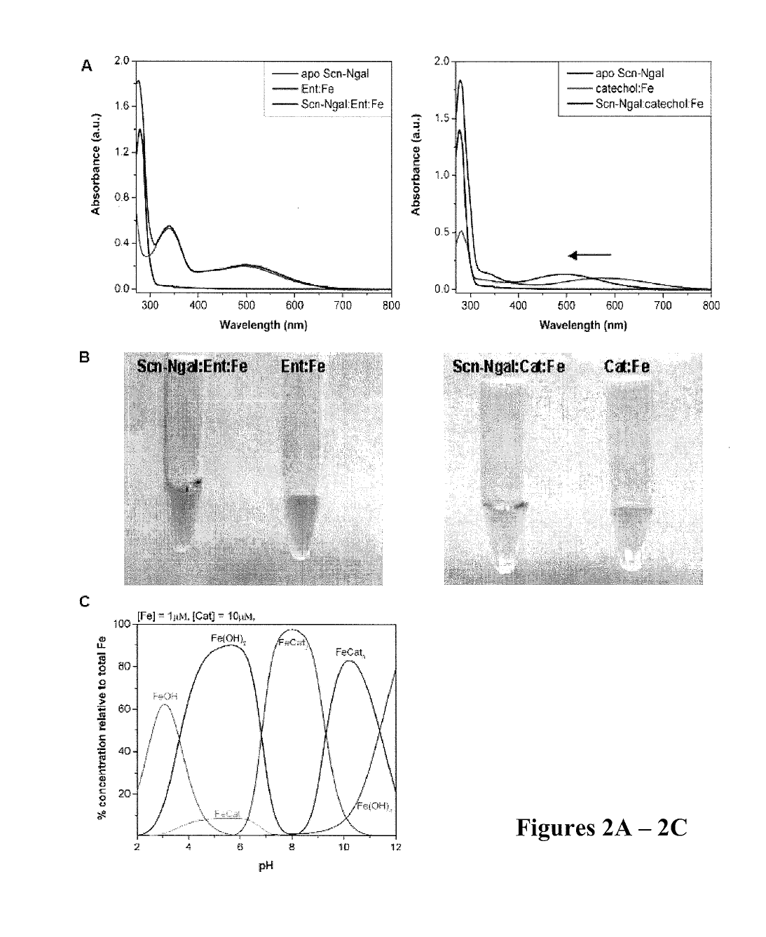

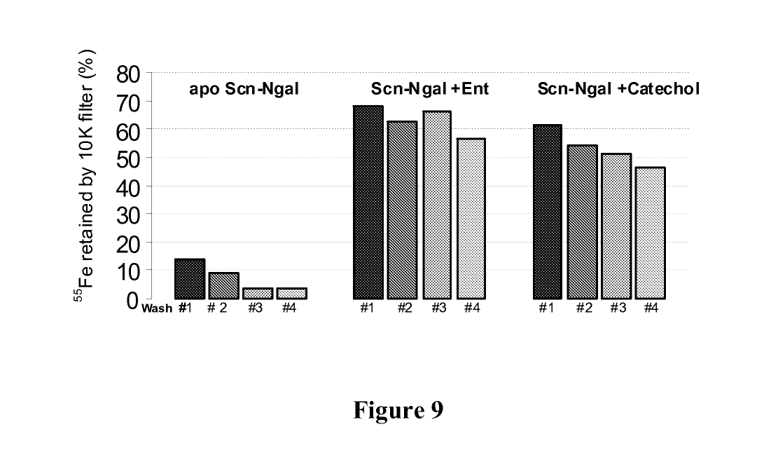

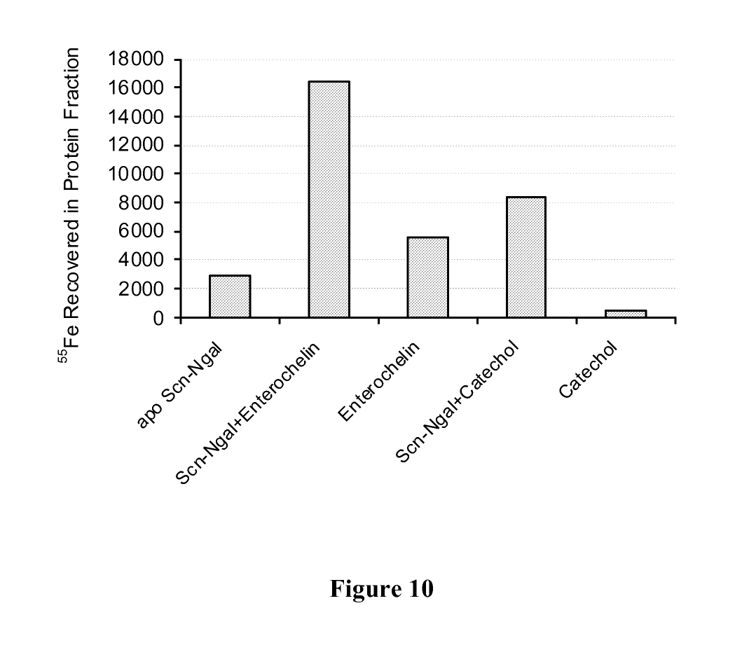

The association of iron with NGAL was examined next. Increasing concentrations of catechol family members resulted in increased and saturable iron binding (FIG. 1B, C). Remarkably, while catechol itself bound with poor affinity (K_{d}=0.20±0.06 mM), nanomolar interactions were detected in the presence of iron (a 10⁵ increase in binding affinity). Binding was best described by two dissociation constants (K_{d1}=2.1±0.5 nM; K_{d2}=0.4±0.2 nM; FIG. 1D, E, F), suggesting a stepwise addition of ligands. Given the predicted stoichiometry of complexes of catechols with iron at pH 7.4 (FIG. 2; Sanchez et al., 2005), a di-catechol:iron (FeL₂) complex was probably recruited first, followed by an additional catechol to generate the optimized hexadentate coordination of iron (FeL₃) within the calyx. The recruitment of the catechols caused a visible spectral shift from blue (FeL₂:λₘₐₓ=575 nm) to red (FeL₃:λₘₐₓ=498 nm), resulting from a decrease in the wavelength of ligand-metal charge-transfer (FIG. 2). This occurs because strong-field catechol ligands (Fe^{III}Lₙ+L=>Fe^{III}Lₙ₊₁) destabilize iron t_{2g} orbitals, increasing the energy-gap between the ligand and metal orbitals involved in the charge transfer, which accounts for the spectral shift to higher energy (Karpishin, R. B., Gebhard, M. S., Solomon, E. I, Raymond, K. N. (1991). J. Am. Chem. Soc. 113, 2977). Hence, the striking increase in affinity for the catechols upon the addition of iron was likely due to the formation of the hexadentate coordination structure (FeL₃) which enhanced both cation-π interactions (FeL₃ contains three catechol groups) as well as Coulombic (FeL₃ is trianionic) interactions in the NGAL calyx. Indeed, the NGAL:catechol:iron complex could survive repetitive washes and gel-filtration chromatography (pH7.0), just as well as Ent or 2,3-dihydroxybenzoate complexes, reflecting roughly similar stabilities (FIGS. 9, 10). Hence, NGAL creates a variety of high affinity catechol:iron complexes, by using electrostatic and cation-π interactions to recruit components of these complexes into the calyx.

### Example 2

### Structural Studies

To define the specific binding site for the catechols, NGAL and catechol:iron were co-crystallized using nearly identical conditions as those used for NGAL:Ent:iron (pH 4.5). The NGAL protein used was that of SEQ ID NO. 2, which was expressed and purified as described in Goetz 2000 and Goetz 2002, the contents of which are hereby incorporated by reference. Structures (dₘᵢₙ=2.3 Å) were determined by direct phasing from a prior structure (PDB accession code 1L6M) and refined to acceptable statistics (Tables 1A-B). While diffraction data were collected from a number of complexes, difference Fourier syntheses showed clear, unambiguous ligand density only for catechol:iron and 4-methylcatechol:iron. The binding of 4-methylcatechol was observed in spite of its low affinity for NGAL, likely due to the high concentration of ligand in these crystallization conditions (FIG. 1D, E, F). None of these ligands significantly affected the overall structure of NGAL when compared with previous structures (PDB accession codes 1DFV, 1QQS, 1L6M, 1X71, 1X89, 1X8U; Goetz, D. H., et al. (2000) Ligand preference inferred from the structure of neutrophil gelatinase associated lipocalin, Biochemistry 39, 1935-1941; Goetz, D. H., et al. (2002) The Neutrophil Lipocalin NGAL Is a Bacteriostatic Agent that Interferes with Siderophore-Mediated Mediated Iron Acquisition, Mol. Cell 10, 1033-1043; Holmes, M. A., et al. (2005) Siderocalin (Lcn 2) also binds carboxymycobactins, potentially defending against mycobacterial infections through iron sequestration, Structure 13, 29-41). For example, pairwise superposition RMSDs between the catechol:iron or 4-methylcatechol:iron complex structures and the NGAL:Ent:iron complex structure (1L6M) were 0.25 Å (catechol) and 0.25 Å (4-methylcatechol) between molecules A, 0.73 Å (catechol) and 0.44 Å (4-methylcatechol) between molecules B, and 0.24 Å (catechol) and 0.23 Å (4-methylcatechol) between molecules C (calculated on all common Cas) in the asymmetric units. Molecule B showed higher disorder, reflected in poorer quality electron density and higher B-factors (Table 1), accounting for the greater disparity among these molecules. Structural conservation extended to residues making direct contact with ligands in the calyx except for residues W79 and R81 which adopted alternate rotamers from those seen in the Ent:iron complex (1L6M).

TABLE D
Crystallographic Statistics

| Data Collection | | | | |
|---|---|---|---|---|
| Ligand | Fe-Catechol | | Fe-4-methyl-Catechol | |
| Space group | P4₁2₁2 | | P4₁2₁2 | |
| Unit cell (Å) | a = b = 115.4, c = 188.8 | | a = b = 114.9, c = 119.6 | |
| Resolution (Å) | 50-2.3 | (2.38-2.3) | 50-2.3 | (2.38-2.3) |
| Rmerge (%) | 0.055 | (0.39) | 0.048 | (0.40) |
| I/σI | 31.5 | (5.09) | 25.0 | (5.39) |
| Redundancy | 7.9 | (6.8) | 8.0 | (7.8) |
| Completeness (%) | 99.9 | (100) | 99.9 | (100) |
| Unique Reflections | 36375 | (3562) | 35984 | (3519) |

| | | |
|---|---|---|
| Refinement | | |
| Statistics | | |
| Rwork (%) | 25.6 | 24.4 |
| Rfree ^{b} (%) | 28.3 | 29.0 |
| Number of atoms | 4057 | 4339 |
| Protein | 3704 | 3973 |
| Ligand | 19 | 32 |
| Water | 243 | 270 |
| Est. Coor. | 0.168 | 0.170 |
| Error (Å) ^{c} | | |
| Geometry | | |
| RMSD Bonds Å | 0.005 | 0.005 |
| RMSD angles (°) | 0.925 | 0.941 |
| RMSD chiral (Å³) | 0.063 | 0.061 |
| Average B (Å²) | 50.4 | 45.5 |
| Protein monomer | 41.5, 88.5, 32.9 | 38.3, 69.0, 32.7 |
| B factors | | |
| (A, B, C; Å²) | | |
| Siderophore B | 56.5, 94.8, 48.0 | 57.6, 86.2, 42.8 |
| factors | | |
| (A, B, C; Å²) | | |
| Water B factors Å² | 47.8 | 47.5 |
| Ramachandran ^{d} | | |
| Most favored (%) | 84.7 | 89.5 |
| Additionally | 12.4 | 8.9 |
| allowed (%) | | |
| Generously | 1.2 | 0.2 |
| allowed (%) | | |
| Disallowed (%) ^{e} | 1.7 | 1.4 |
| PDB Accession | 3FW4 | 3FW5 |
| Code | | |

^{a} Numbers in parentheses correspond to the highest resolution shells
^{b} Calculated on 10% of the data (Kleywegt, G. J., Brugner, A. T. (1996). “Checking your imagination: applications of the free R value”, Structure 15, 897-904) and matched between the original structure PDB code 1L6M
^{c} Based on maximum likelihood in refmac ([0044] Murshudov, G. N., Vagin, A. A., Dodson, E. J. (1997). Refinement of Macromolecular Structures by the Maximum-Likelihood Method. Acta Cryst. D53, 240-255).
^{d} Calculated with PROCHECK ([0037] Laskowski, R. A., MacArthur, M. W., Moss, D. S, Thornton, J. M. (1993). PROCHECK: a program to check the stereochemical quality of protein structures. J. Appl. Cryst., 26, 283-291).
^{e} The Ramachandran outliers generally occur in the same two residues (Y115 and C175). These same residues are observed outliers in other NGAL structures at higher resolution, arguing that these residues are not poorly modeled but truly adopt unfavorable conformations.

TABLE E
Crystallographic Statistics

| Data Collection | | | | | | | | |
|---|---|---|---|---|---|---|---|---|
| Ligand | Fe-3-methyl- | | Fe-Pyrogallol | | Fe-Caffeic Acid | | Fe-Rosmarinic | |
| | Catechol | | | | | | Acid | |
| Space group | P4₁2₁2 | | P4₁2₁2 | | P4₁2₁2 | | P4₁2₁2 | |
| Unit cell (Å) | a = b = 115.1, | | a = b = 116.3, | | a = b = 114.42, | | a = b = 114.81, | |
| | c = 118.6 | | c = 120.8 | | c = 119.15 | | c = 118.70 | |
| Resolution (Å) | 50-3.25 | (3.37-3.25) | 50-2.7 | (2.8-2.7) | 50-2.43 | (2.52-2.43) | 35-2.4 | (2.53-2.44) |
| Rmerge (%) | 0.135 | (0.39) | 0.063 | (0.35) | 0.051 | (0.28) | 0.057 | (0.27) |
| I/σI | 7.28 | (2.96) | 23.9 | (6.69) | 15.2 | (7.5) | 21.4 | (5) |
| Redundancy | 3.5 | (3.4) | 5.3 | (5.1) | 14.1 | (11.4) | 14.4 | (14.2) |
| Completeness (%) | 98.1 | (100) | 95.5 | (99.5) | 99.5 | (95) | 99.9 | (98.9) |
| Unique Reflections | 12861 | (1284) | 22259 | (2249) | 30330 | (2824) | 30337 | (2937) |

| | | | | |
|---|---|---|---|---|
| Refinement | | | | |
| Statistics | | | | |
| Rwork (%) | — | — | — | — |
| Rfree ^{b} (%) | — | — | — | — |
| Number of atoms | — | — | — | — |
| Protein | — | — | — | — |
| Ligand | — | — | — | — |
| Water | — | — | — | — |
| Est. Coor. | — | — | — | — |
| Error (Å) ^{c} | | | | |
| Geometry | | | | |
| RMSD Bonds Å | — | — | — | — |
| RMSD angles (°) | — | — | — | — |
| RMSD chiral (Å³) | — | — | — | — |
| Average B (Å²) | — | — | — | — |
| Protein monomer | — | — | — | — |
| B factors | | | | |
| (A, B, C; Å²) | | | | |
| Siderophore B | — | — | — | — |
| factors | | | | |
| (A, B, C; Å²) | | | | |
| Water B factors Å² | — | — | — | — |
| Ramachandran ^{d} | | | | |
| Most favored (%) | — | — | — | — |
| Additionally | — | — | — | — |
| allowed (%) | | | | |
| Generously | — | — | — | — |
| allowed (%) | | | | |
| Disallowed (%) ^{e} | — | — | — | — |
| PDB Accession | — | — | — | — |
| Code | | | | |

^{a} Numbers in parentheses correspond to the highest resolution shells
^{b} Calculated on 10% of the data (Kleywegt and Brugner, 1996) and matched between the original structure PDB code 1L6M
^{c} Based on maximum likelihood in refmac ([Murshudov, G. N., Vagin, A. A., Dodson, E. J. (1997). Refinement of Macromolecular Structures by the Maximum-Likelihood Method. Acta Cryst. D53, 240-255).
^{d} Calculated with PROCHECK (Laskowski et al., 1993).
^{e} The Ramachandran outliers generally occur in the same two residues (Y115 and C175). These same residues are observed outliers in other NGAL structures at higher resolution, arguing that these residues are not poorly modeled but truly adopt unfavorable conformations.

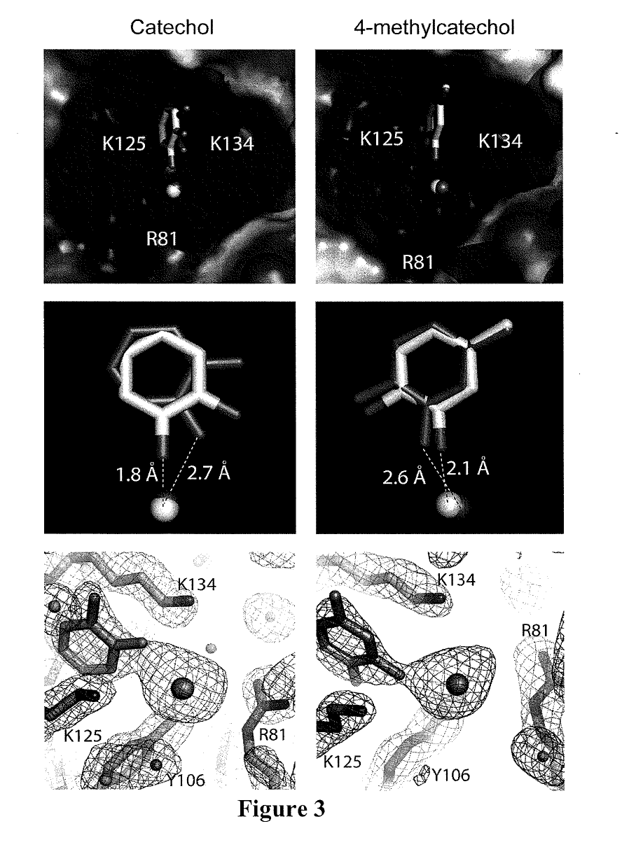

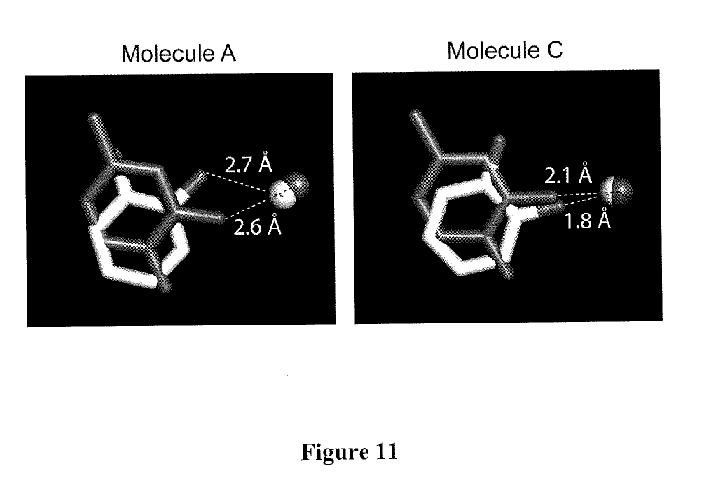

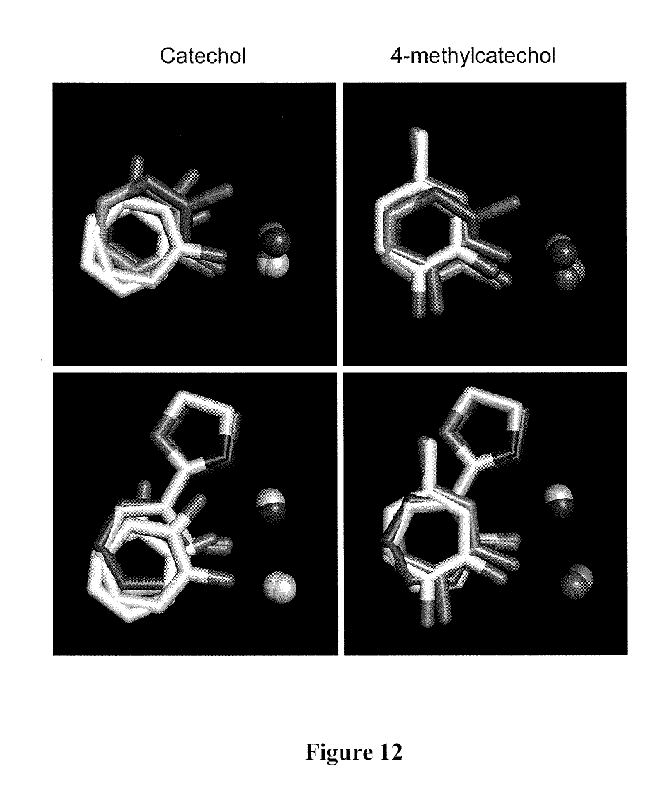

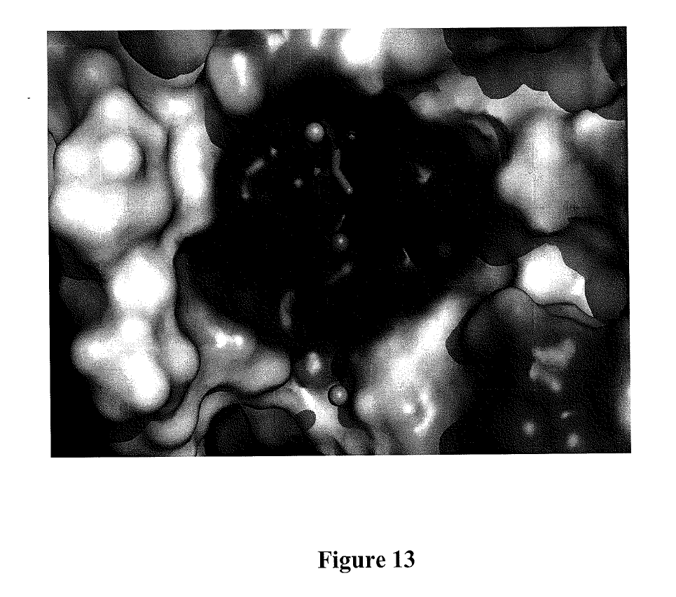

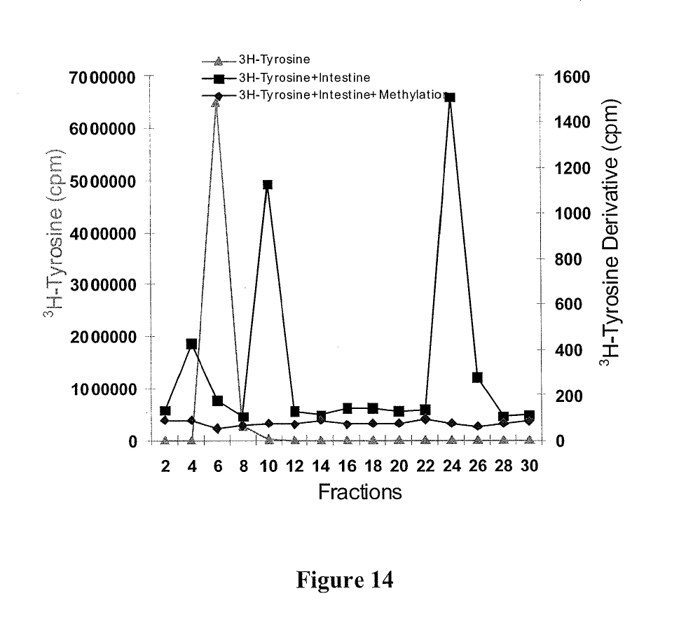

A single catechol or 4-methylcatechol occupied pocket #1 of NGAL (Goetz et al., 2000; Goetz et al., 2002) between the side-chains of residues K125 and K134 (FIG. 3C). Compared with catechol, 4-methylcatechol was rotated so that the hydroxyl groups faced down into the calyx and the ligand was shifted upwards (˜1 Å) out of the calyx; this shift accommodated the methyl substitution and relieved steric clashes in pocket #1 (FIG. 3, FIG. 11). With the exception of the rotation of the catechols around the axis perpendicular to the rings, they superimposed with the phenyl groups of Ent and Cmb in corresponding NGAL complex structures (FIG. 12).

Both catechol and 4-methylcatechol were found to coordinate iron, but as a result of the crystallization at pH 4.5 (see below), or alternatively as a result of the oxidation of catechol to semiquinone, iron was coordinated by only one catechol hydroxyl group (FIG. 3), the second facing out of the calyx, resulting in partial occupation of iron sites. The lack of full hexacoordination of iron also created a net positive charge, which was compensated by the variable binding of chloride atoms in the calyx (FIG. 13). These data showed that pocket #1 determined ligand specificity by contributing the highest affinity for polarized aryl groups found in catechol:iron complexes.

### Example 3

### Traffic of NGAL:Catechol:Iron

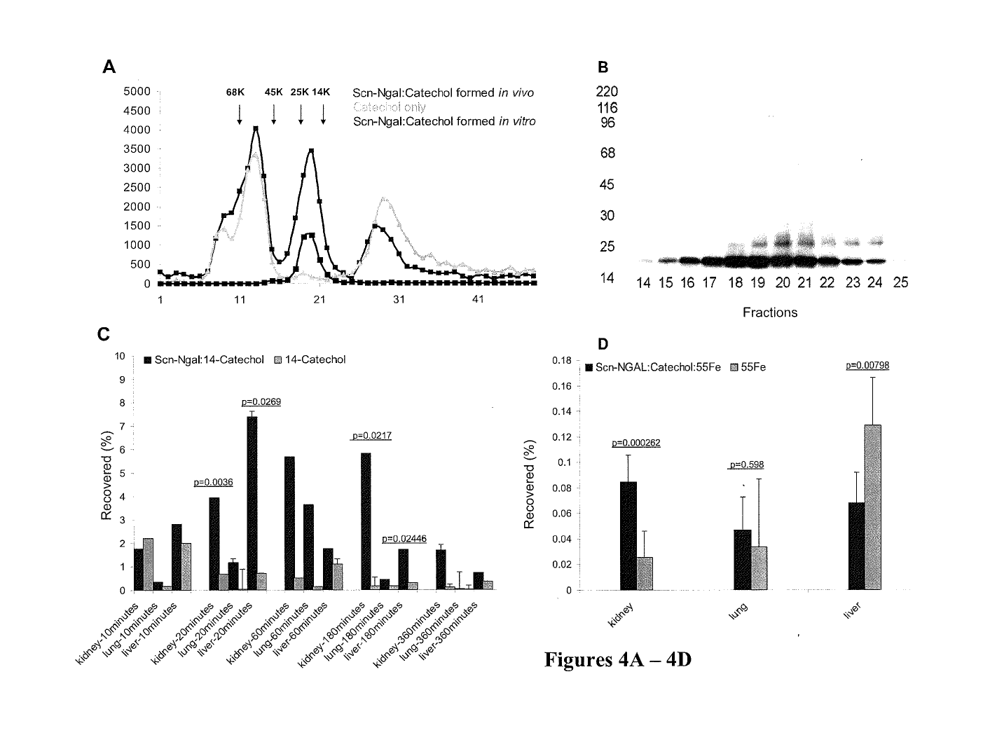

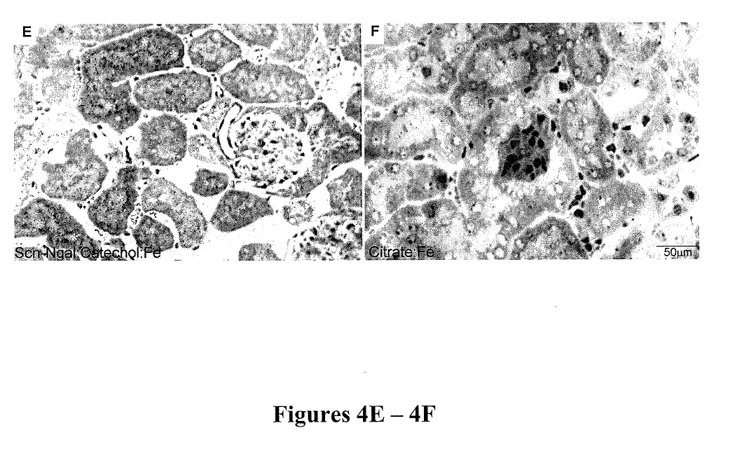

To test whether NGAL:catechol solubilized iron and permitted its transport in vivo, initial experiments demonstrated that when introduced separately in a mouse, NGAL and catechol formed a complex in the plasma (FIG. 4A, B). The complex then distributed to a number of organs, but particularly to the kidney (kidney>liver, p<0.05 at 180 min.), where it enhanced the delivery of catechol (in kidney, NGAL:catechol>catechol, p<0.01 and p<0.05 at 20 and 180 min., respectively; FIG. 4C) as well as iron (in kidney, NGAL:catechol:iron>iron, p<0.001; FIG. 4D). The iron accumulated in the kidney and could be visualized at the apical membrane of the proximal tubule by radioautography (FIG. 4E, F). As a control, citrate-bound iron, which is not bound by NGAL, was used. Citrate:iron targeted the liver instead of the kidney (in liver, citrate:iron>NGAL:catechol:iron, p<0.01). Hence, the NGAL:catechol complex can transport iron in vivo.

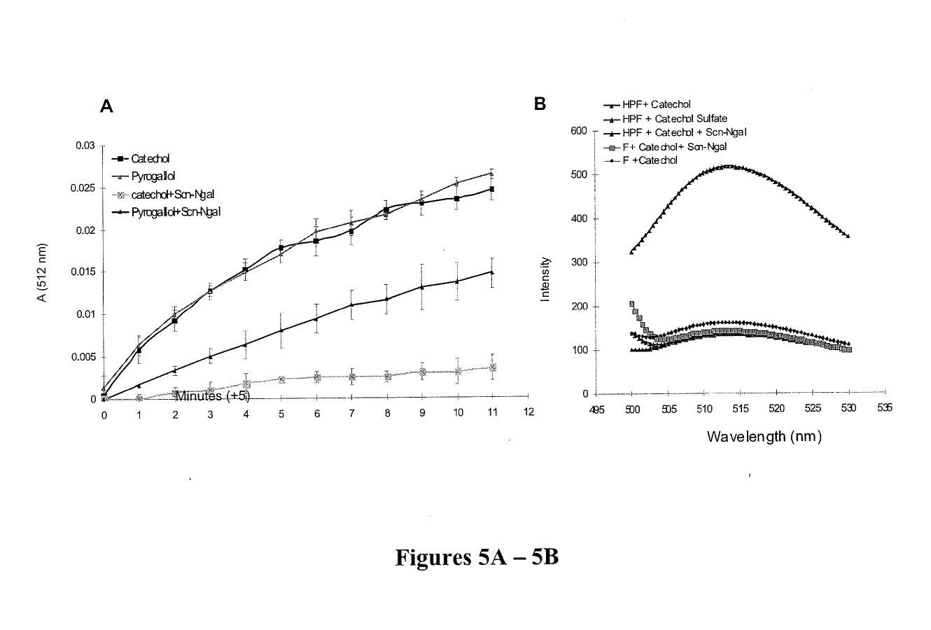

Effective chelation of iron should not only result in its solubilization, but also in limiting its reactivity. Catechols activate the Fenton reaction (Rodriguez, J., Parra, C., Contreras, F. J., Baeza, J. (2001). Dihydroxybenzenes: driven Fenton reactions. Water Sci Technol. 44, 251-256; Iwahashi, H., Morishita, H., Ishii, T., Sugata, R., Kido, R. (1989). Enhancement by catechols of hydroxyl-radical formation in the presence of ferric ions and hydrogen peroxide. J. Biochem. 105, 429-434) by reducing iron (Fe³⁺=>Fe²⁺) and thereby accelerating hydroxyl-radical formation. These data were confirmed using catechol, pyrogallol, 3-methylcatechol or 4-methylcatechol. However, the addition of stoichiometric quantities of NGAL (NGAL:catechol 1:3, respectively) inhibited the detection of phenanthroline reactive Fe^{2|} (catechol: p<0.001; pyrogallol: p<0.01; FIG. 5A). NGAL also blocked the conversion of the 3′-(p-hydroxyphenyl) fluorescein (HPF) to fluorescein (p<0.001), which was induced by catechol:Fe in the presence of H₂O₂ (FIG. 5B), but the protein did not directly affect the fluorescence of a flourescein control molecule (Setsukinai, K., Urano, Y., Kakinuma, K., Majima, H. J., Nagano, T. (2003). Development of Novel Fluorescence Probes That Can Reliably Detect Reactive Oxygen Species and Distinguish Specific Species. J Biol Chem. 278, 170-3175). Blocking the catechol hydroxyl groups by O-sulfonation or O-methylation prevented the reduction of iron as well as the generation of radicals, demonstrating the specificity of these assays. These data demonstrate that by sequestering catechol:iron complexes, NGAL limits iron reactivity in Fenton type reactions.

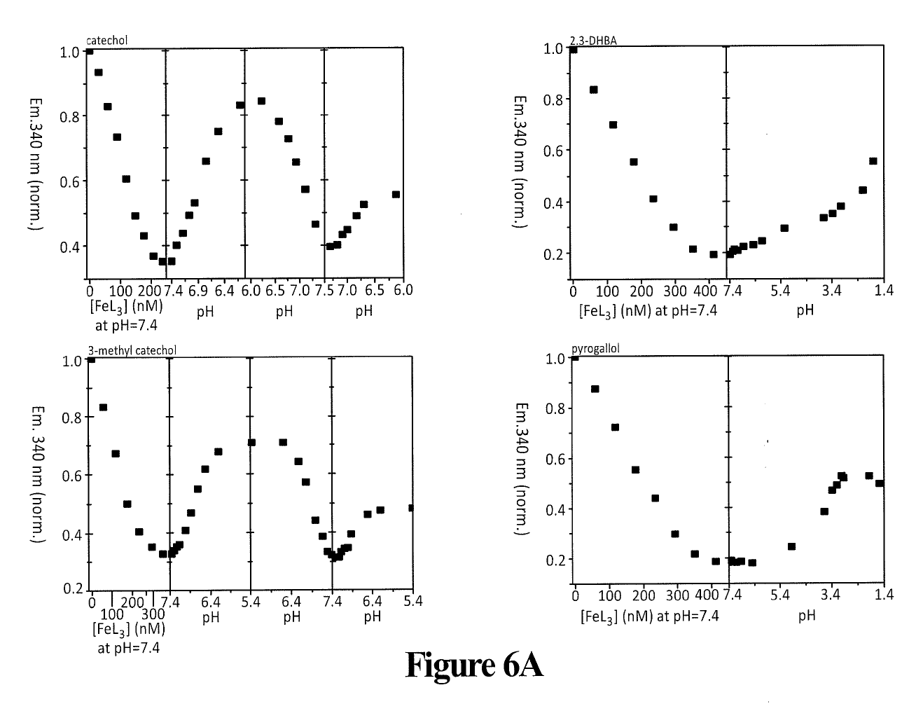

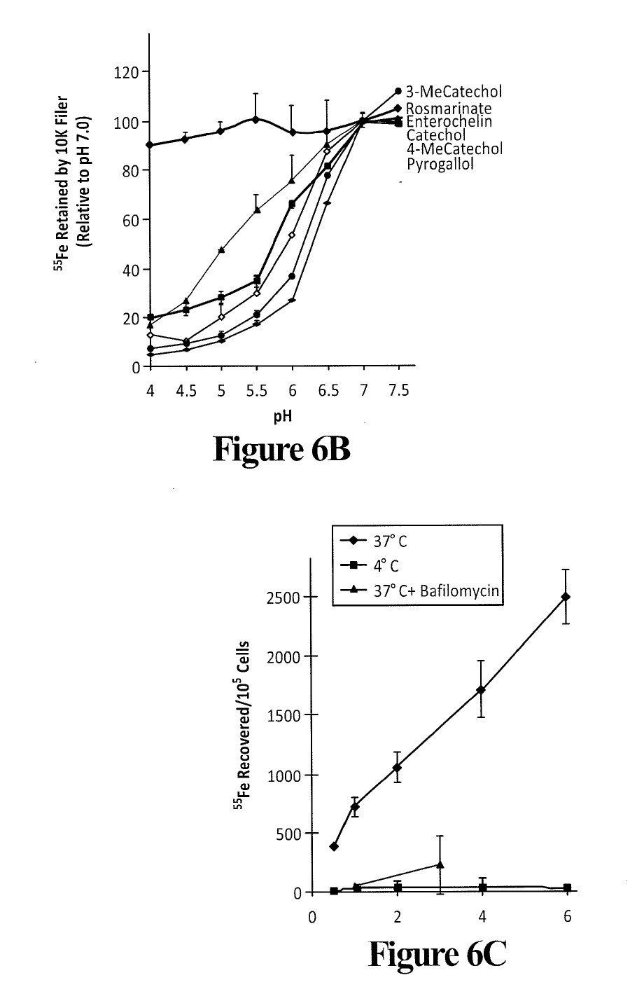

To transport iron from plasma to cells there has to be a mechanism for releasing iron from the complex. Intracellular delivery of iron by transferring is known to require passage through acidified endosomes where iron is released. Experiments were designed to test whether a similar mechanism existed for NGAL:catechol:iron complexes. While the complex was stable at neutral pH, acidification below pH 7.0 progressively reversed ligand-dependent fluorescence quenching of NGAL and released iron (FIG. 6A, B). The catechol and 3-methylcatechol complexes were nearly completely dissociated by pH 5.5, while pyrogallol and 2,3-dihydroxybenzoate complexes did not dissociate until below pH 4. Acid dependent dissociation was likely due to protonation of the catechol hydroxyls or alternatively amino acids within the calyx, since NGAL remained correctly folded upon acidification (Abergel, R. J., Clifton, M. C., Pizarro, J. C., Warner, J. A., Shuh, D. K., Strong, R. K., Raymond, K. N. (2008). The Siderocalin/Enterobactin Interaction: A Link between Mammalian Immunity and Bacterial Iron Transport. J. Am. Chem. Soc. 130, 11524-11534). These data are consistent with pH-mediated dissociation of FeL₃ to FeL (Sanchez, P., Gálvez, N., Colacio, E., Miñones, E., Domínguez-Vera, J. M. (2005). Catechol releases iron (III) from ferritin by direct chelation without iron (II) production. Dalton Trans. 4:811-813) and the stages of binding that were apparent from the multiphasic fluorescence quenching data. The pH sensitivity also explained the presence of a single catechol in the pH 4.5 crystal, implying that the crystal structures represented the final stages of pH-mediated ligand release.

To test the relevance of the pH sensitivity of the NGAL:catechol:iron complex in a biologically relevant assay, the radiolabeled complex was added to cells, and demonstrated iron donation. This process was then blocked when the vacuolar H⁺ ATPase of endocytic vesicles was inhibited with bafilomycin, or when endocytosis was blocked by incubation at 4° C., indicating that iron donation required trafficking of the complex into intracellular acidified compartments (FIG. 6C). In fact, the catechol complex delivered 3 times more iron than the Ent:iron complex which required non-physiologic acidity or reduction of its iron for complete dissociation (Mori et al., 2005; Abergel et al., 2008). Hence, unlike Ent, the binding properties of the catechols ideally matched the physiological requirements for the transport and delivery of iron to cells. This same mechanism may apply to the proximal tubule in the kidney, where megalin dependent endocytosis removed the NGAL complex from the filtrate and directed the complex to acidified endosomes (Mori et al., 2005, FIG. 4E).

### Example 4

Source Of Catechols

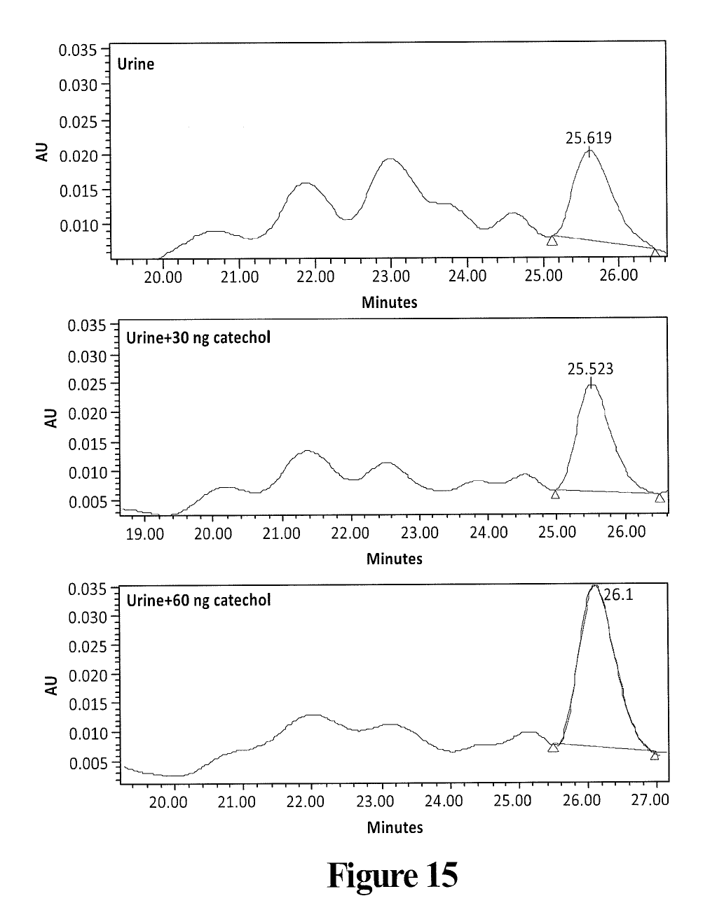

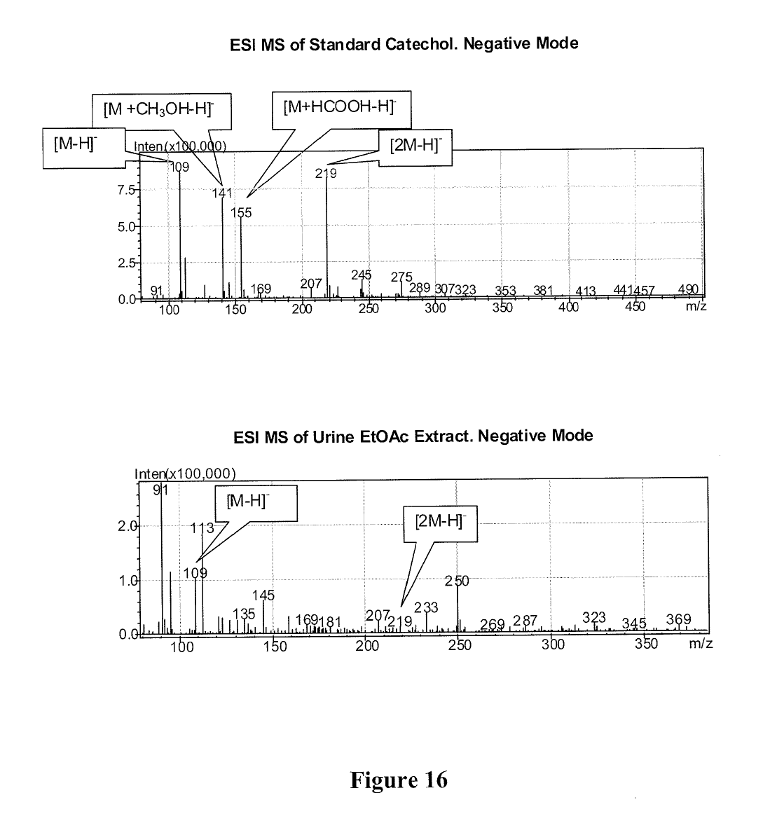

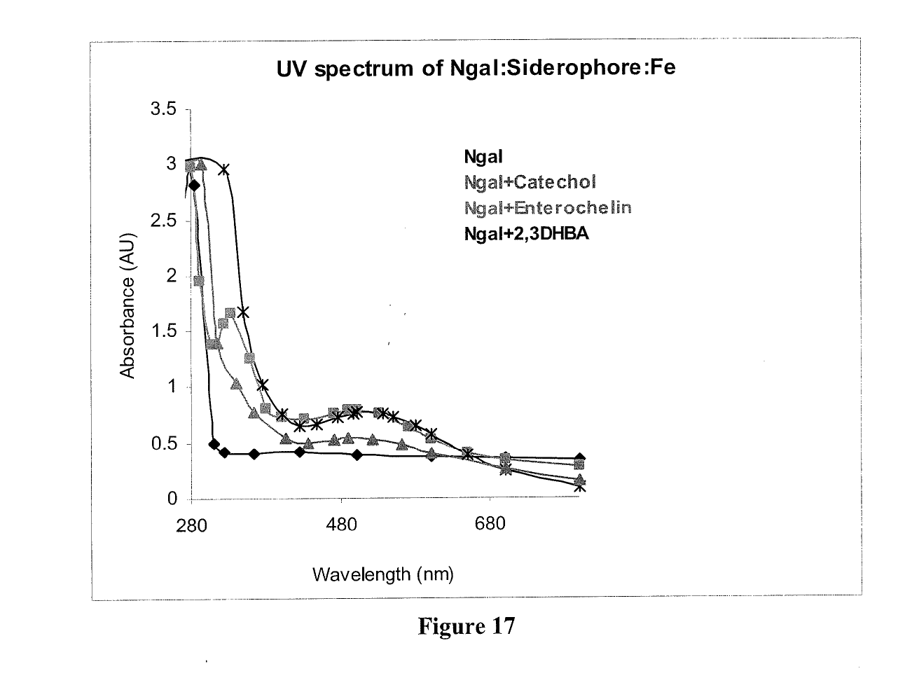

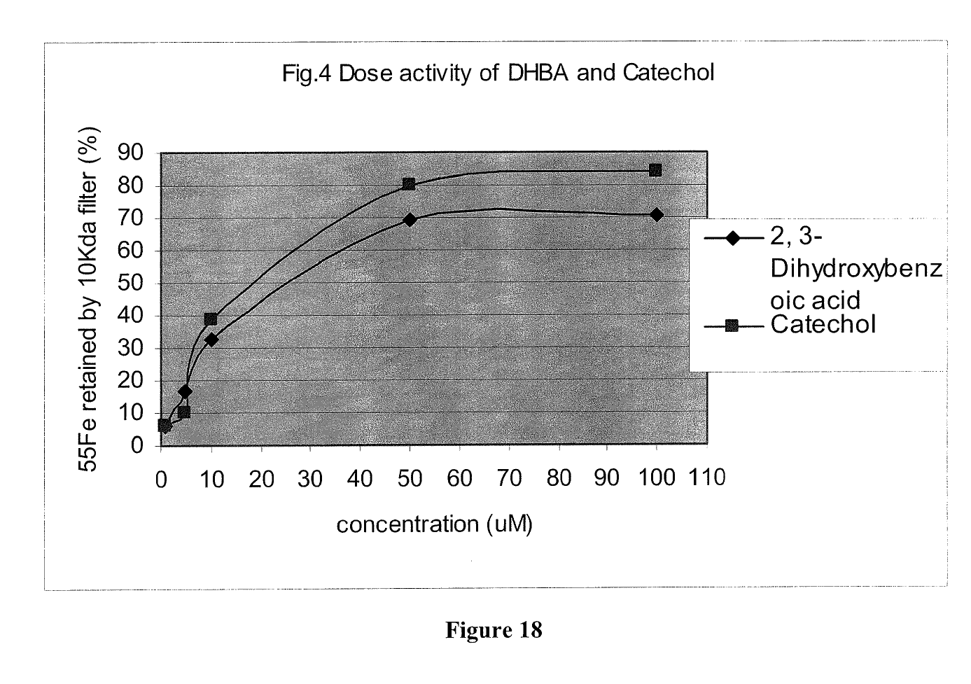

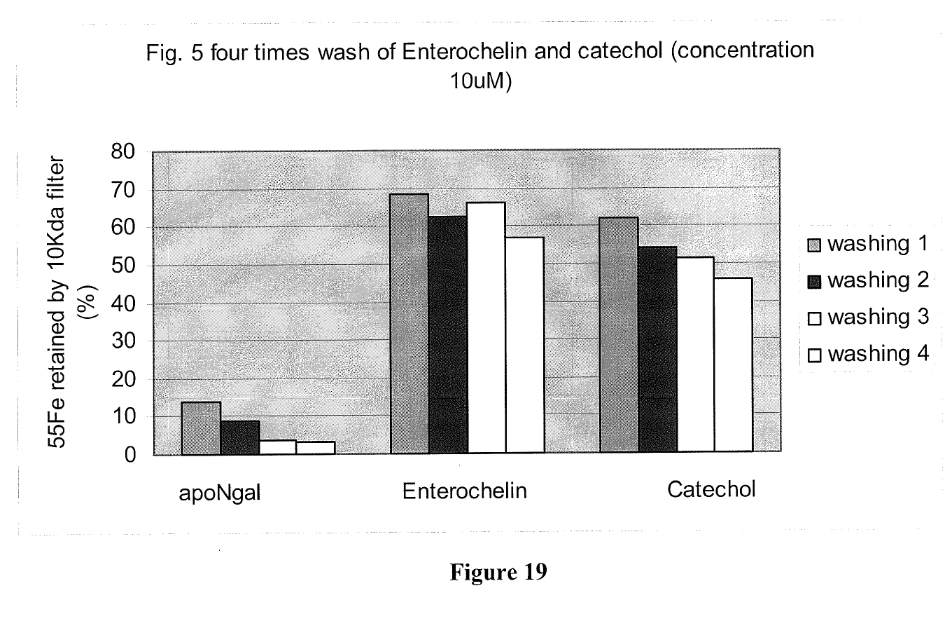

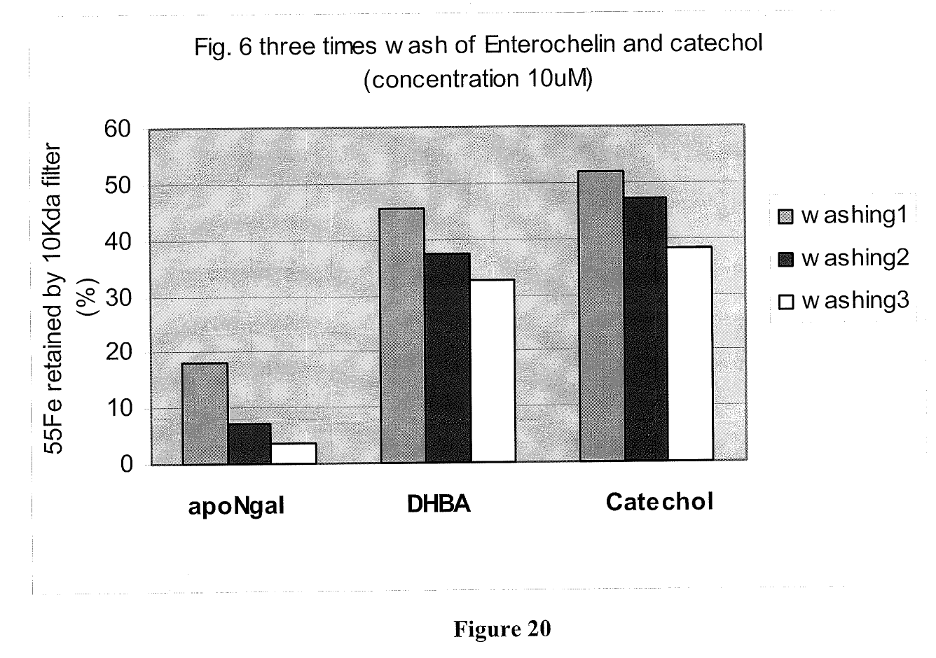

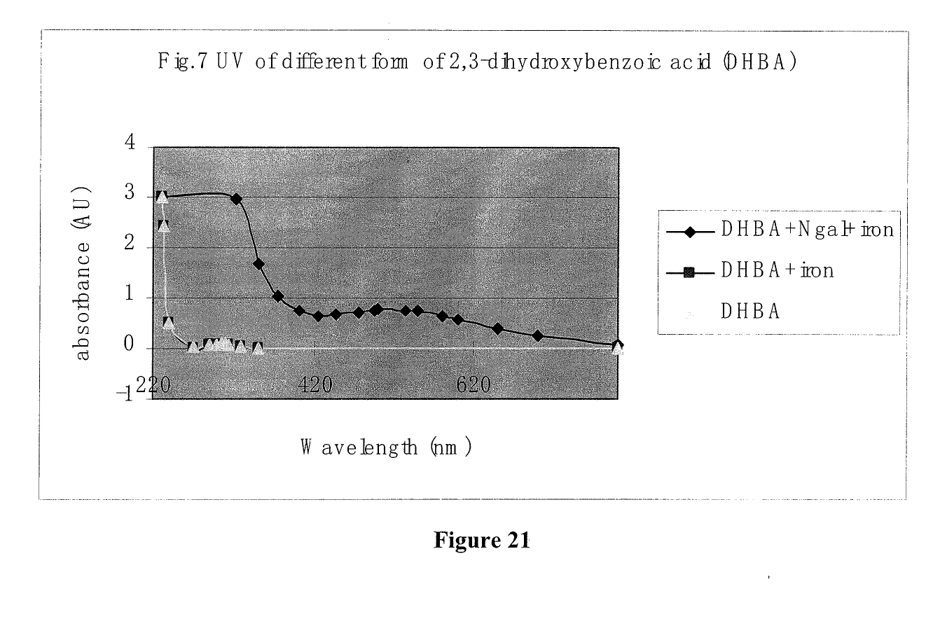

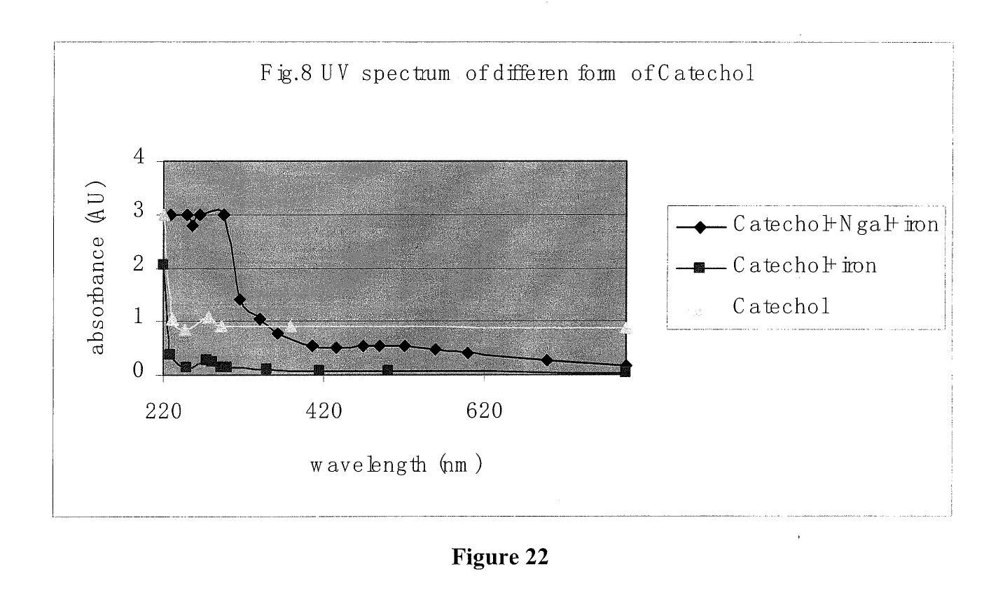

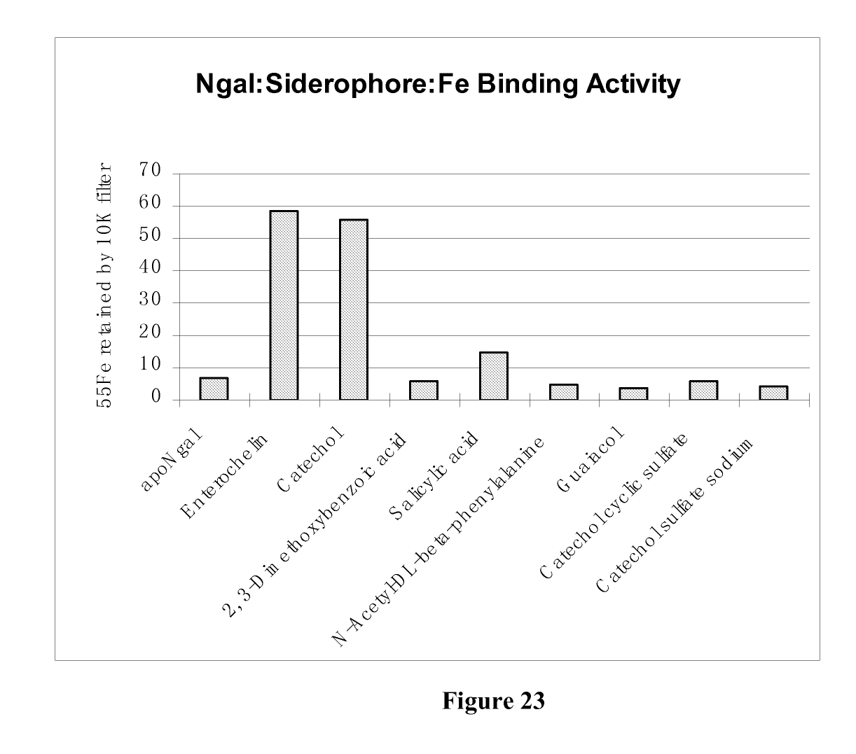

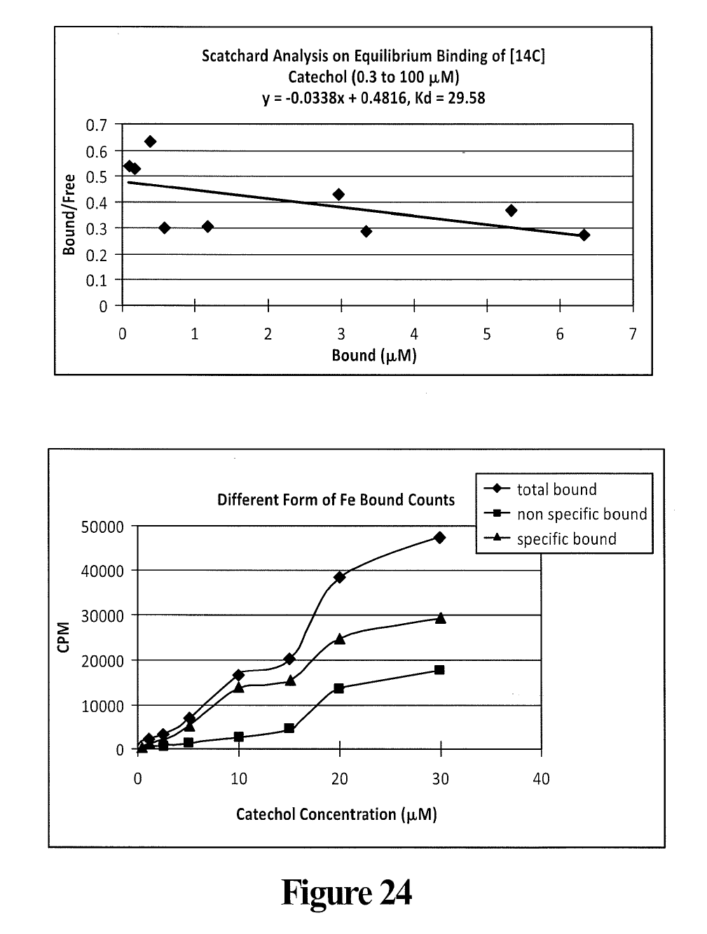

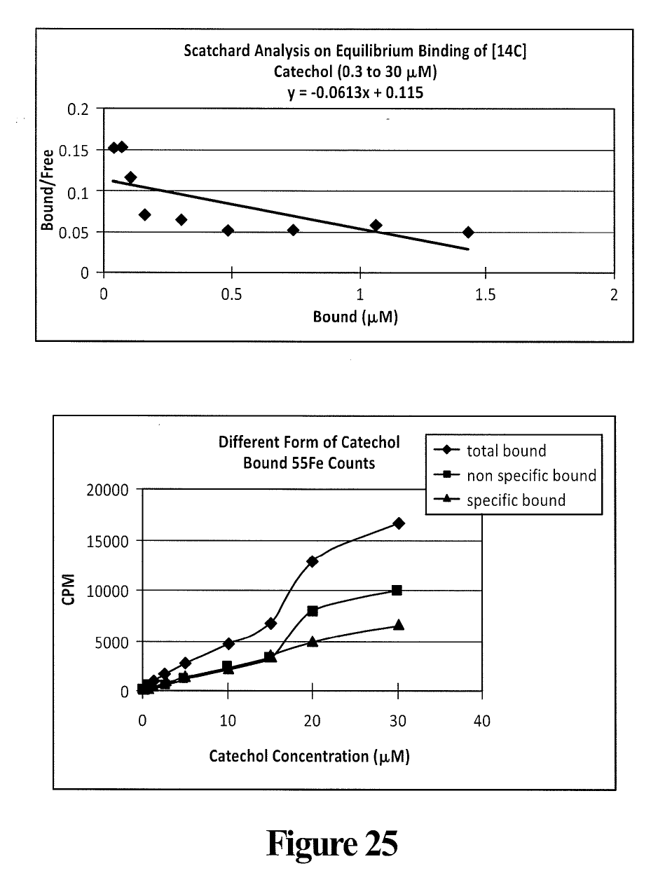

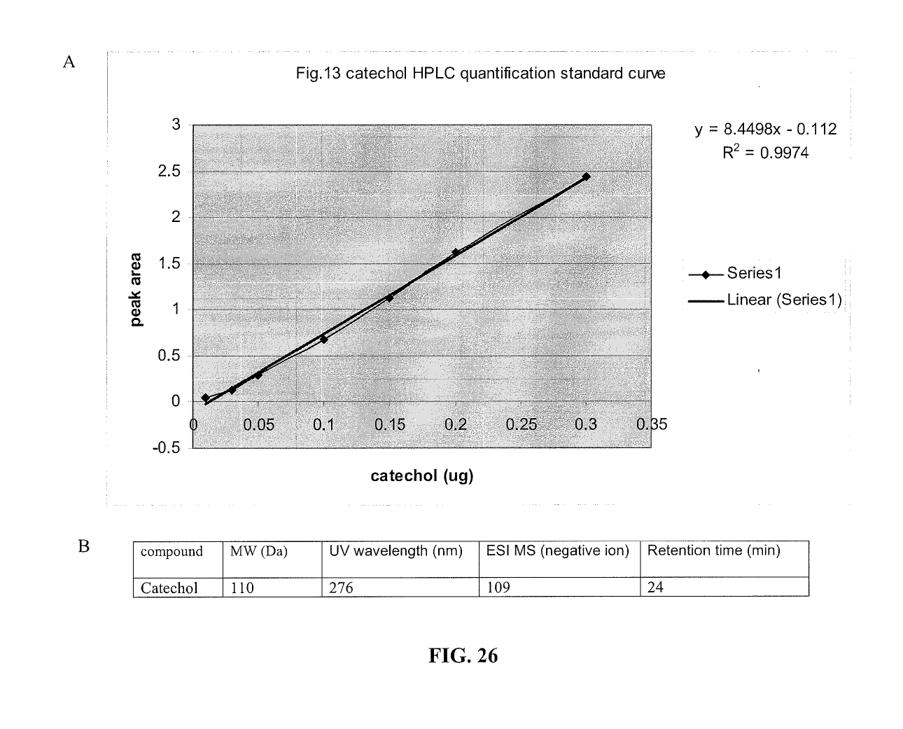

Catechols are abundant metabolites in mammals, where they are derived from polyphenols (˜50%; quinic and shikimic acids; Booth, A. N., Robbins, D. J., Masri, M. S., DeEds, F. (1960). Excretion of catechol after ingestion of quinic and shikimic acids. Nature 187, 691; Martin, A. K. (1982). The origin of urinary aromatic compounds excreted by ruminants. 3. The metabolism of phenolic compounds to simple phenols. Br. J. Nutr. 48, 497-507; Lang, R., Mueller, C., Hofmann, T. (2006). Development of a stable isotope dilution analysis with liquid chromatography-tandem mass spectrometry detection for the quantitative analysis of di- and trihydroxybenzenes in foods and model systems. J. Agric Food Chem. 54, 5755-5762; Carmella, S. G., La, V. E. J., Hecht, S. S. (1982). Quantitative analysis of catechol and 4-methylcatechol in human urine. Food Chem Toxicol. 20, 587-590) and aromatic amino acids (˜50%; Martin et al., 1982; Carmella et al., 1982; Bakke, O. M. (1969). Urinary simple phenols in rats fed purified and nonpurified diets. J. Nutr. 98, 209-216). The existence of the latter pathway was confirmed by mixing ³H-tyrosine with lung and particularly the intestine (but not spleen, liver or heart; FIG. 7) which resulted in the generation of ³H-catechol. The precise source of circulating mammalian catechols, or catechols found in other mammalian fluids (such as urine) or cells, is not known, but it might result from a combination of bacterial and mammalian metabolism, since oral antibiotics suppressed the level of urine catechol in our studies (˜50%; Martin et al., 1982; Smith, A. A., (1961). Origin of urinary pyrocatechol. Nature 190, 167). Hepatic enzymes have also been shown to be involved in the production of the catechols (Sawahata, T., Neal, R. A. (1983). Biotransformation of phenol to hydroquinone and catechol by rat liver microsomes. Mol. Pharmacol. 23, 453-460). Eventually, the catechols are excreted in the urine in large quantities [catechol (20-30 μM) FIG. 15; 4-methylcatechol (30 μM); pyrogallol (500 μM); Martin et al., 1982; Lang et al., 2006; Carmella et al., 1982; Bakke et al., 1969; Kim, S., Vermeulen, R., Waidyanatha, S., Johnson, B. A., Lan, Q., Rothman, N., Smith, M. T., Zhang, L., Li, G., Shen, M., Yin, S., Rappaport, S. M. (2006). Using urinary biomarkers to elucidate dose-related patterns of human benzene metabolism. Carcinogenesis 27, 772-781), whereupon they are sulfonated (Rennick, B., Quebbemann, A. (1970). Site of excretion of catechol and catecholamines: renal metabolism of catechol. Am. J. Physiol. 218, 1307-1312). Sulfonation blocked the catechol:iron interaction (Table B), but nonetheless, urinary free catechol (1-5% of the total; Booth et al., 1960; Martin et al., 1982) remained in a concentration ten-fold that of peak levels of NGAL. In addition, free catechol was stable in solution (as monitored for 20 hrs by NMR; D₂O: 6.9 ppm, ²H; 6.7 ppm, ²H) and readily detectable by ESI mass spectroscopy in the urine (109 m/z peak; negative mode) which was authenticated by methylation using mass detection (FIG. 16) as well as TLC. In sum, the catechols are abundant metabolic products in mammals that derive from components of food in part by the metabolic actions of microorganisms.

### Example 5

### Experimental Methods

Chemicals. Urinary compounds (Table B; http://www.hmdb.ca/) were obtained commercially (all of the compounds illustrated in Table A are commercially available from Sigma-Aldrich; HPLC grade solvents (Fisher); Catechol-¹⁴C (100 mCi/mmol; Sigma); L-[5-³H]Tryptophan (32.0 Ci/mmol), L-[4-³H]Phenylalanine (27.0 Ci/mmol), and L-[3,5-³H]Tyrosine (54.0 Ci/mmol) GE Healthcare; ⁵⁵FeCl₃ (PerkinElmer); Enterochelin (Ent; EMC Microcollections); TLC plates and chromatography paper (Whatman). NGAL was expressed in BL-21 bacteria (Yang, J., Goetz, D., Li, J. Y., Wang, W., Mori, K., Setlik, D., Du, T., Erdjument-Bromage, H., Tempst, P., Strong, R., Barasch, J. (2002). An iron delivery pathway mediated by a lipocalin. Mol Cell 10, 1045-1056). Human urine was pooled from healthy medical school students and patients with IRB approval. Mouse urine was obtained from CD1 mice and from CD1 mice treated with oral Vancomycin and Neomycin for 1 week prior to urine collection (Bioreclamation).

Instruments. Ultrospec 3300 pro UV/Visible Spectrophotometer (Amersham Biosciences); ESI Mass (Shimadzu 2010 LC-MS); ¹H-NMR spectra (Varian-300 mHz instrument in CD₃OD). Fluorescence quenching (Cary Eclipse fluorescence spectrophotometer).

HPLC Analysis. Urine was filtered (0.22 μm), extracted 3 times with ethyl acetate and the residue taken up in methanol and analyzed by HPLC and ESI-MS (in negative mode). In some cases, the sample was hydrolyzed (HCl×90 min, 100° C.). Analytical work (Waters 996) utilized a 2.1 mm×150 mm, i.d., 3.5 μm beads, C18 SunFire Column with eluant A (0.5% acetic acid in methanol) and eluant B (0.5% acetic acid in water) at a flow rate of 0.5 mL/min. Eluant A was increased linearly to 8% within 5 min, 15% within 45 min, and then 100% within 5 min, followed by 100% for 10 min. Authentic catechol standardized the results.

To quantify the metabolites of L-[5-³H]Tryptophan, L-[4-³H]Phenylalanine, and L[3,5-³H]Tyrosine, samples were spiked with catechol (10 μg), extracted with EtOAc, and analyzed on a Waters 600 HPLC, Perkin-Elmer LC-95 UV/Visible spectrophotometer detector and a 4.6 mm×150 mm, i.d., 3.5 μm beads, C-18 SunFire Column with the same eluants as above. The catechol peak was collected and quantified by HPLC-UV (catechol (x) vs Area Under Curve (y): y=5.4206x-0.0141; R²=0.998; limit of detection 0.1 μg/ml).

Fluorescence quenching binding assay. Excitation λₑₓₑ=281 nm (5 nm slit band pass) and emission λₑₘ=340 nm (10 nm slit band pass) data were collected from 100 nM protein solutions (with 32 μg/mL ubiquitin and 5% DMSO) exposed to ligands. To prepare FeL₃, catechol (12 mM, 25 μL in DMSO) and ferric chloride (0.33 eq.) were combined and then diluted to form the metal complex 18 μM FeL₂ (in iron) in aqueous buffer (pH 7.4; TBS) and 5% DMSO. Apo-catechol (“apo” refers to an iron-free molecule) solutions were prepared analogously. The pH was adjusted until the fluorescence signal stopped changing, while fluorescence values were corrected for dilution. Data were analyzed by a nonlinear regression analysis using a one-site binding model (Kuzmic, P. (1996). Program DYNAFIT for the analysis of enzyme kinetic data: application to HIV proteinase. Anal. Biochem. 237, 260-273). Control experiments were performed to ensure the stability of the protein at experimental conditions, including the dilution and the addition of DMSO and ubiquitin.

Computational Methods. Computational studies were conducted at the Molecular Graphics and Computation Facility, College of Chemistry, University of California, Berkeley. To determine the quadrupole moments, Θ_{zz} the aromatic structures were geometry optimized and characterized via frequency calculations at the RHF/6-311G** level of theory in the Gaussian 03 package (Frisch, M. J. T. et al. (2004). In Gaussian 03, Revision C.02; Gaussian, Inc.: Wallingford Conn.). To determine the aromatic-cation interaction energies the components were characterized via a frequency calculation at the MP2/6-311++G** level of theory and the aromatic-cation interaction energies corrected for basis set superposition error (BSSE) with the counterpoise method in the Gaussian 03 package. In the aromatic-cation calculations the sodium ion was fixed at a distance of 2.47 Å above the centroid of the aromatic unit.

Crystallization. Recombinant C87S human NGAL was expressed and purified as previously described (Goetz et al., 2002; Holmes et al., 2005). Protein (10 mg/ml) was mixed with 10 mM catechol or 4-methylcatechol and then with 5 mM FeCl₃, using extensive washes (YM-10, Millipore) with PNE (25 mM PIPES, 150 mM NaCl, and 1 mM EDTA). Co-crystals of ligand bound human NGAL were grown by vapor diffusion at 18° C. over reservoirs of 1.0-1.4 M NH₄SO₄, 100 mM NaCl, 50 mM LiSO₄, 100 mM Na acetate (pH 4.5). Crystals typically grew in 5-10 days and were cryo-protected using the mother liquor plus 15% glycerol prior to flash cooling in LiqN₂. Diffraction data were collected using synchrotron radiation at the Advanced Light Source (Berkeley, Calif.) beamline 5.0.1 (wavelength 1.0λ) and then processed with HKL2000 software (Otwinowski, Z., Minor, W. (1997). “Processing of X-ray Diffraction Data Collected in Oscillation Mode”, Methods in Enzymology, Volume 276: Macromolecular Crystallography, part A, C. W. Carter, Jr. & R. M. Sweet, Eds. (Academic Press New York), pp. 307-326) and the Collaborative Computational Project 4 suite of programs (1999). Initial difference Fourier phases were calculated directly from the NGAL:Ent:iron complex (1L6M) and refined using Refmac (Murshudov, G. N., Vagin, A. A., Dodson, E. J. (1997). Refinement of Macromolecular Structures by the Maximum-Likelihood Method. Acta Cryst. D53, 240-255; Laskowski, R. A., MacArthur, M. W., Moss, D. S, Thornton, J. M. (1993). PROCHECK: a program to check the stereochemical quality of protein structures. J. Appl. Cryst., 26, 283-291) reflections used to calculate R_{free} (Kleywegt, G. J., Brugner, A. T. (1996). “Checking your imagination: applications of the free R value”, Structure 15, 897-904). Models were rebuilt using Coot (Emsley, P., Cowtan, K. (2004). Coot: Model-Building Tools for Molecular Graphics, Acta Cryst. D60, 2126-2132). Relevant statistics are shown in Table 1 and coordinates have been deposited in the Protein Data Bank: www.rcsb.org (Accession Codes: Catechol=3FW4; 4-methylcatechol=3FW5).

Siderophore:Iron Binding Assay. To test whether candidates bound iron directly, candidates (1 nmole) were mixed with ⁵⁵Fe (1 pmole), and then separated bound from free ⁵⁵Fe using paper chromatography developed in water.

NGAL:Siderophore:Iron Binding Assays. Compound-dependent iron binding to apoNGAL (10 μM) was assayed using 150 mM NaCl, 20 mM Tris (pH 7.4), ⁵⁵Fe (1 μM+cold FeCl₃ 9 μM) and candidate compounds (10 μM) at room temperature. After 60 minutes, the mixture was washed 3 times (YM-10) or alternatively gel filtered (PD-10, GE Biosciences). Ent loaded NGAL served as a positive control. Ferric citrate (1 mM) or Ent:iron (500 μM) served as competitors of ⁵⁵Fe binding.

NGAL Blocks Iron Induced Hydroxyl Radicals. Catechol mediated reduction of Fe³⁺=>Fe²⁺ was detected with phenanthroline (Sigma) using citrate as an iron donor (Iwahashi et al., 1989). Hydroxyl radicals were detected by the conversion of HPF to fluorescein (Setsukinai, K., Urano, Y., Kakinuma, K., Majima, H. J., Nagano, T. (2003). Development of Novel Fluorescence Probes That Can Reliably Detect Reactive Oxygen Species and Distinguish Specific Species. J Biol Chem. 278, 170-3175) using as an iron donor.

Capture of NGAL:Siderophore:Iron. Mouse stromal cells (10⁵) were grown in MEM, 10% FCS for 24 hrs and FCS subsequently removed. NGAL:Ent:⁵⁵Fe or NGAL:Catechol:⁵⁵Fe (1:3:1) were added to cells along with bafilomycin (0.15 nmole). Cells were washed 3 times and extracted with 0.2% SDS.

Catechol and Iron Traffic to the Kidney. Liver, kidney, lung were harvested from 1-6 hrs after “C-Catechol, NGAL: “C-Catechol or NGAL:Catechol:⁵⁵Fe (1:3:1) were introduced. Tissues were dissolved in 2%SDS, 0.1N NaOH at 60° C.

Synthesis of Catechol Cyclic Sulfonate. The method of Dubois and Stephenson, 1980, was used (DuBois, G., Stephenson, R. A. (1980). Sulfonylamine-mediated sulfamation of amines. A mild, high yield synthesis of sulfamic acid salts. J. Org. Chem. 45, 5371-5373). Briefly, catechol (1.1 g) in pyridine/hexane (1.6 g/10 ml) was treated with sulfuryl chloride/hexane (1.36 grams/2 ml) at −5° C. overnight, after which the reaction was warmed for 6 hrs. The upper layer was decanted, and the lower layer washed twice with

EtOAc. The combined washes and the upper layer were then washed with 5% Cu(OAC)₂ H₂O, and the absence of catechol demonstrated by TLC (hexane-EtOAc, 3:1) (Rf=0.14). The solution was then dried and recrystallized (colorless needles, mp 35-36° C., lit.s mp 34-35° C.). ¹H NMR (300 MHz, CD₃OD, δ ppm): 7.31 (2H, m), 7.24(2H, m), (catechol: 6.24 and 6.41 ppm). To synthesize catechol sulfonate (Kaiser, E. T., Zaborsky, O. R. (1968). Hydrolysis of esters of sulfur-containing acids in oxygen-18 enriched media. J. Am. Chem. Soc. 90, 4626-4628) catechol cyclic sulfonate (100 mg) was hydrolyzed in acetonitrile, 0.1 N NaOH (1.2 ml/1 ml) for 3 hr, and then extracted with chloroform and dried with ethanol. ¹H NMR (300 MHz, CD₃OD, δ ppm): 7.27 (1H, dd, J=8.1, 1.8 Hz), 7.04 (1H, td, J=7.8, 1.5 Hz), 6.79 (1H, dd, J=8.1, 1.5 Hz), 6.62 (1H, td, J=7.8, 1.5 Hz).

### Example 6

### Catechol Characterization

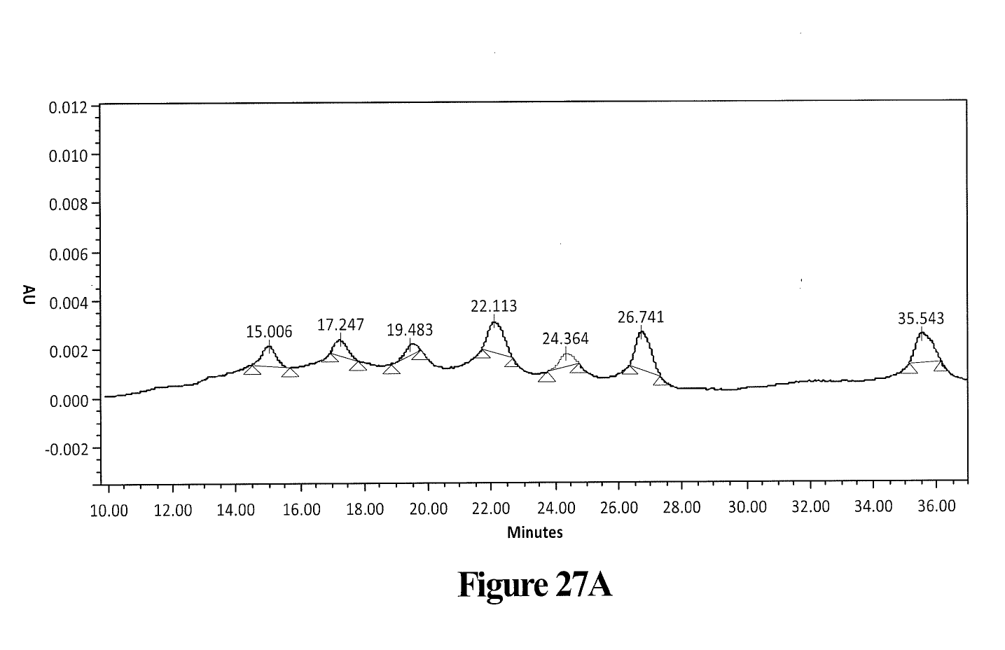

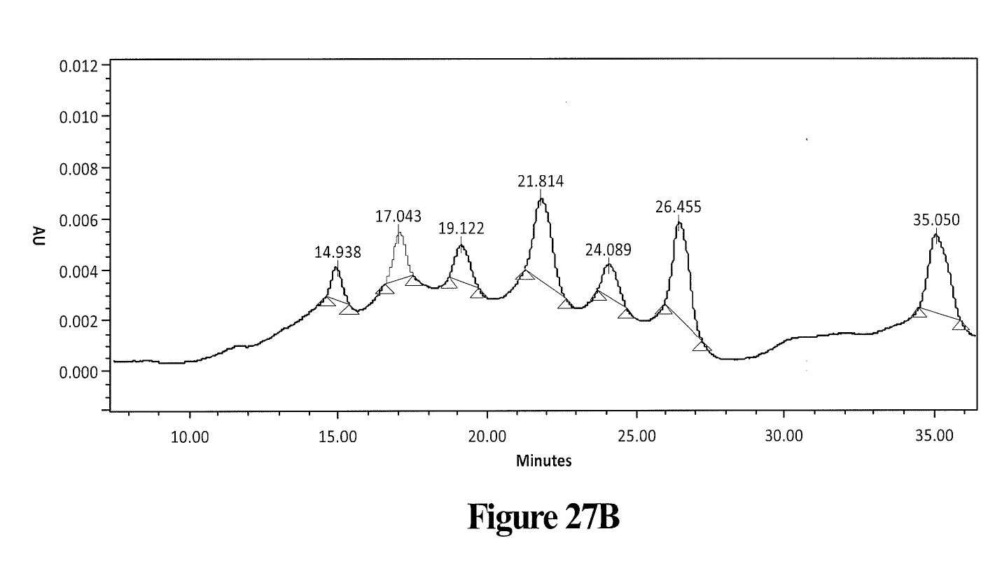

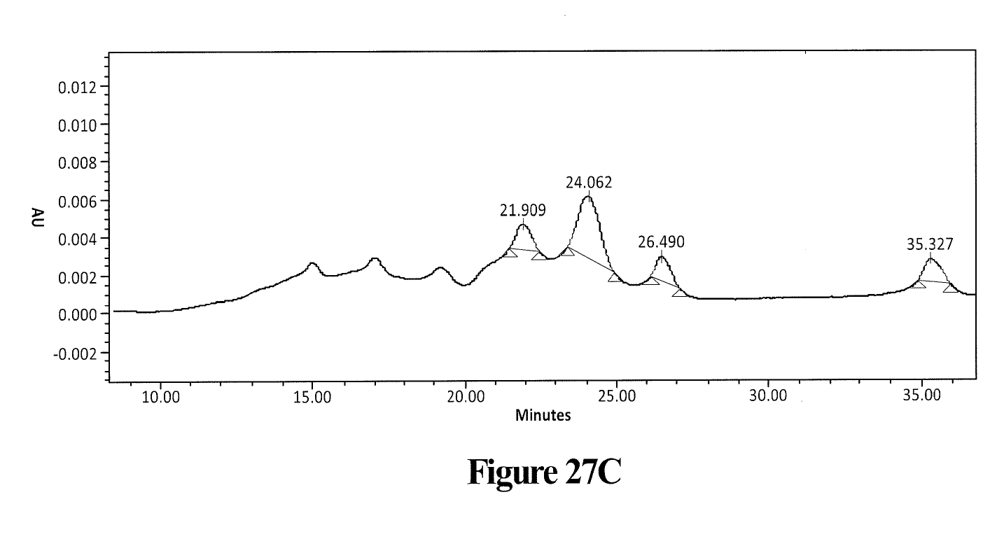

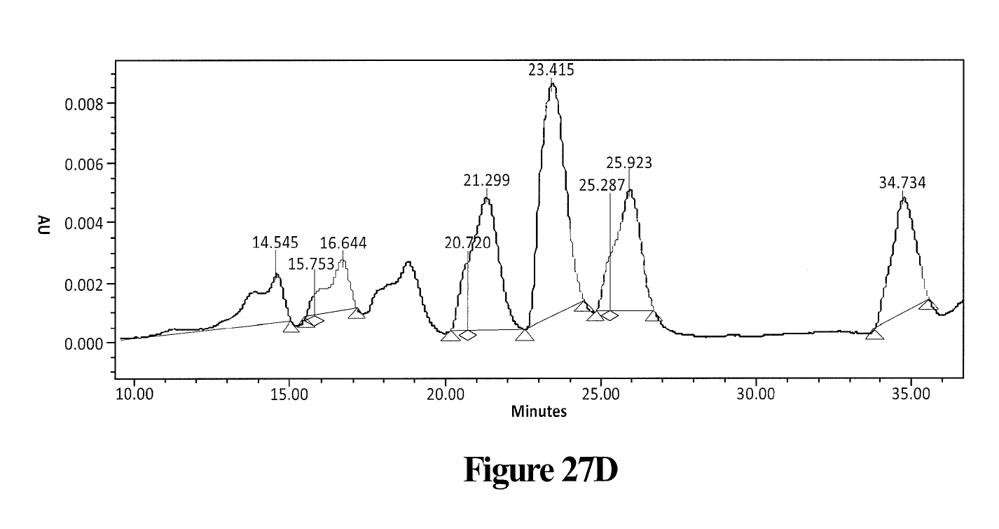

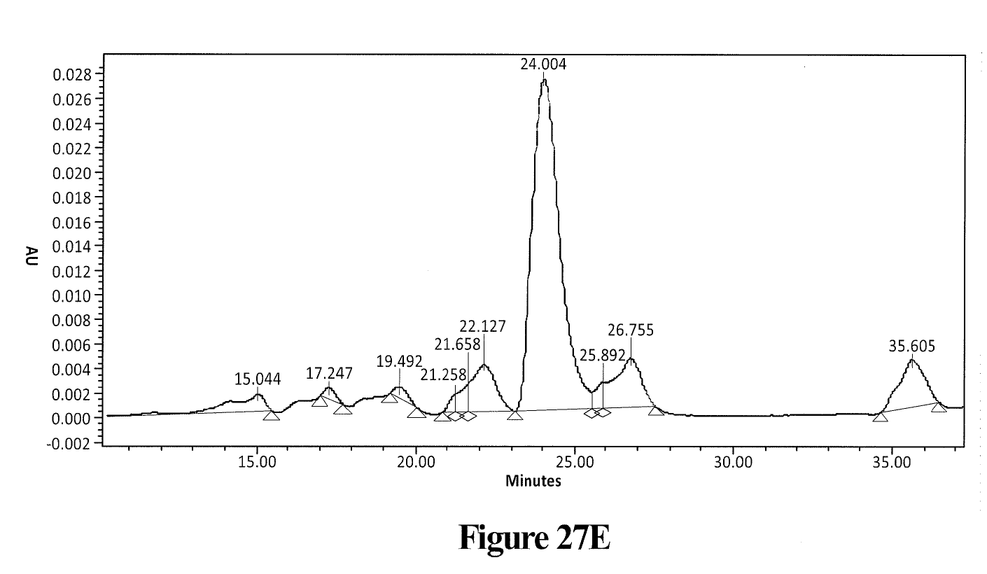

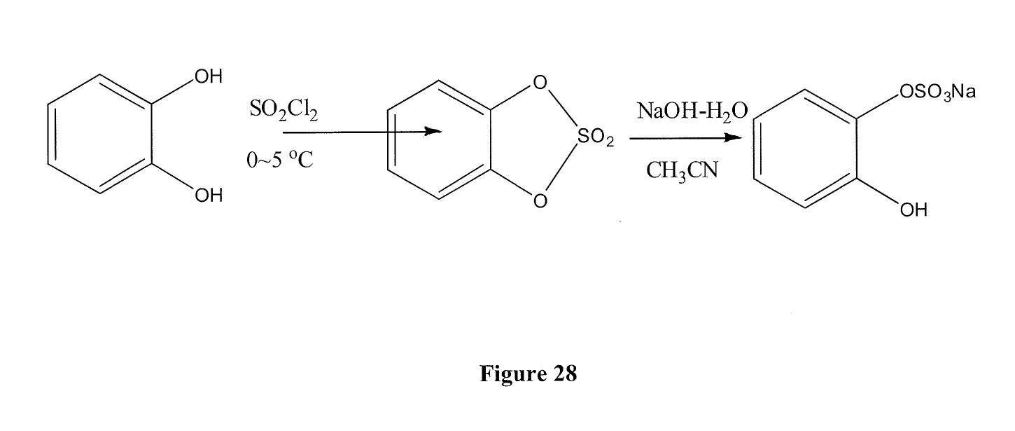

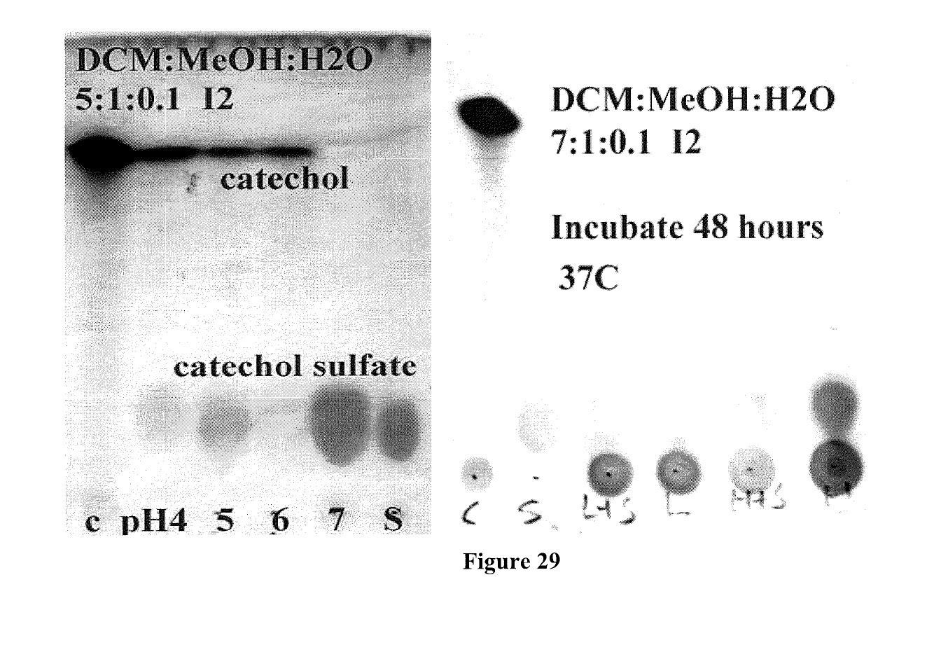

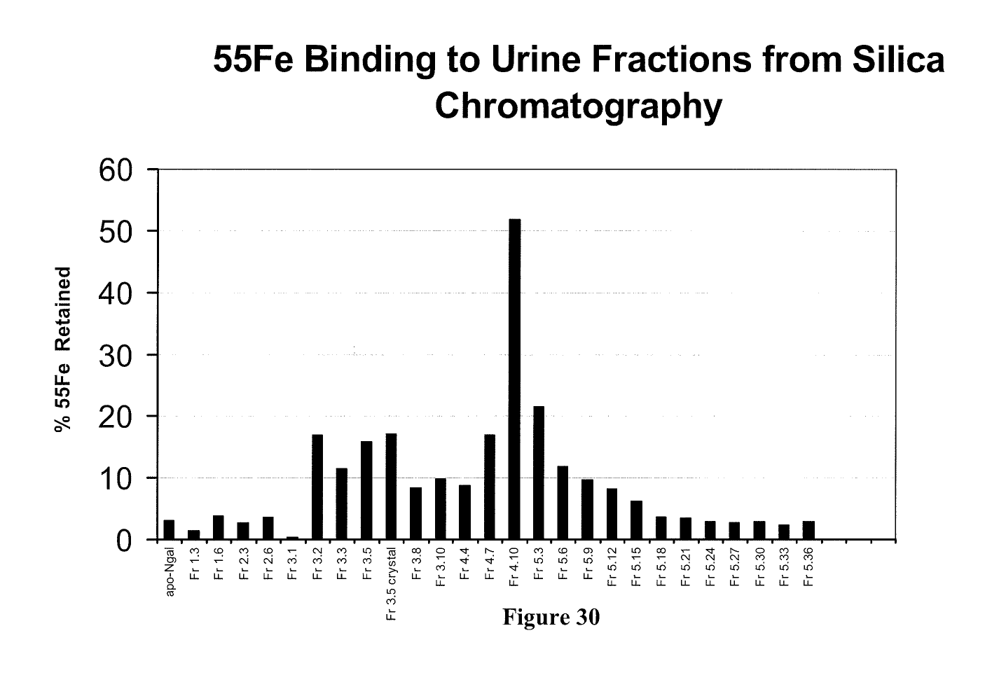

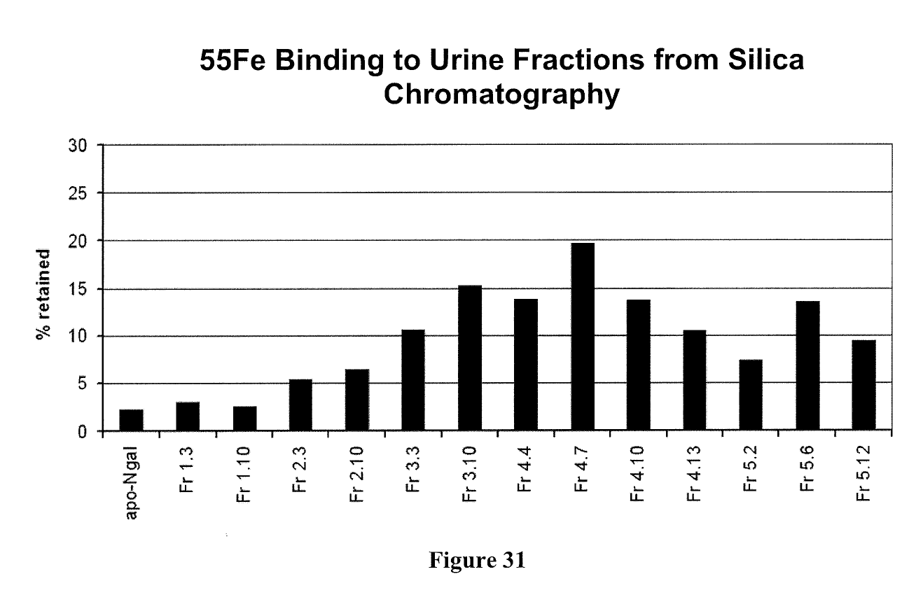

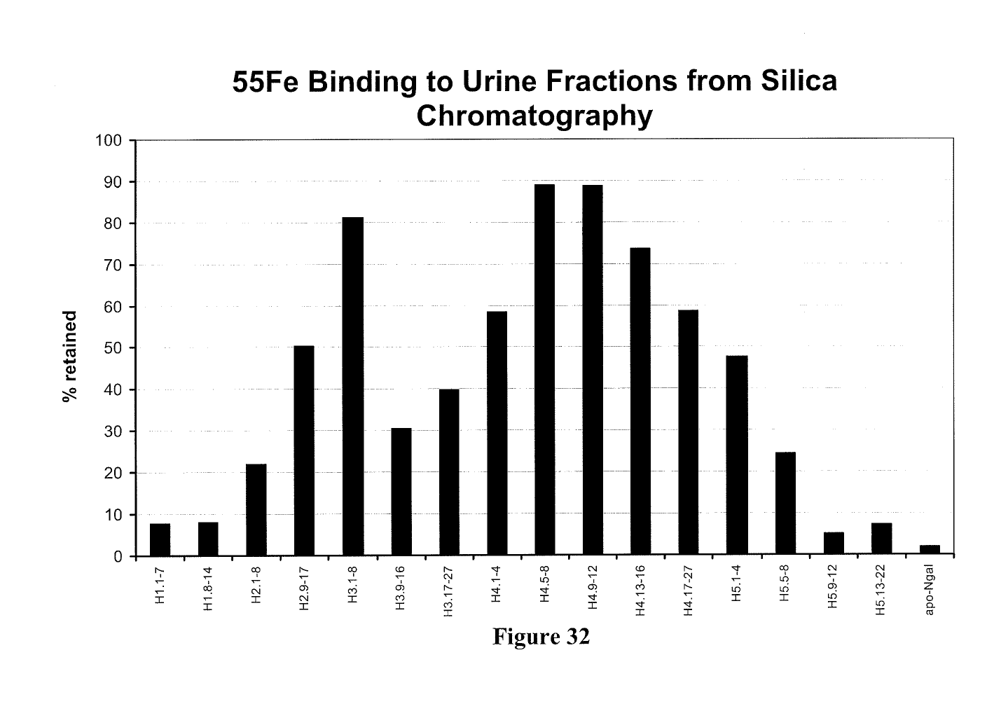

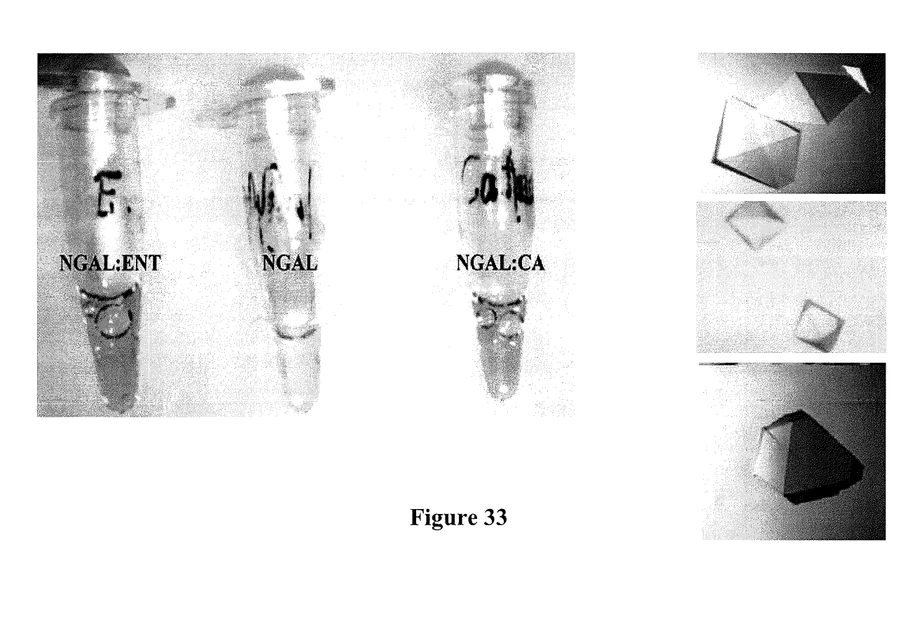

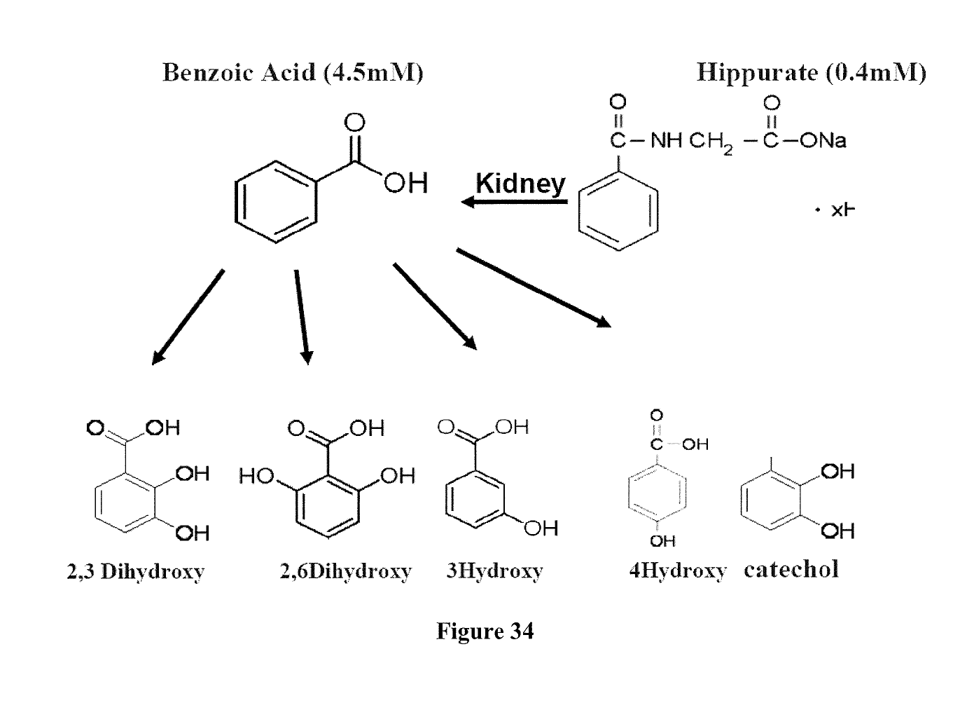

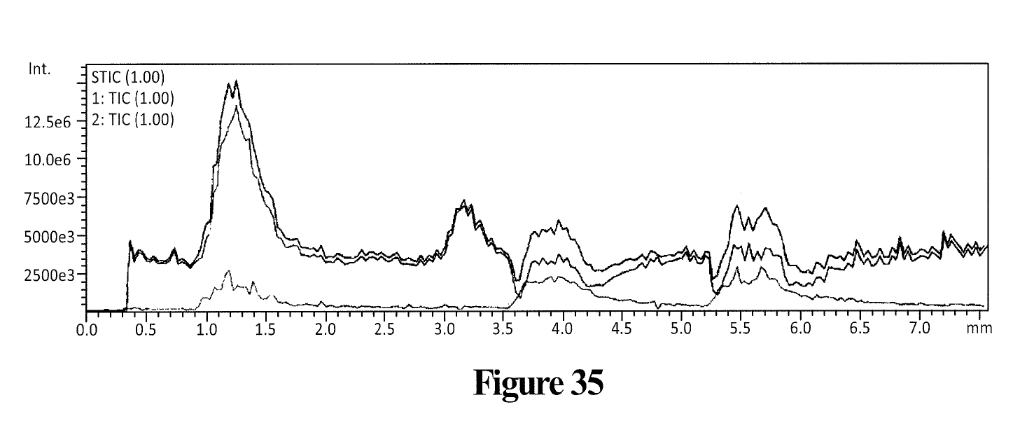

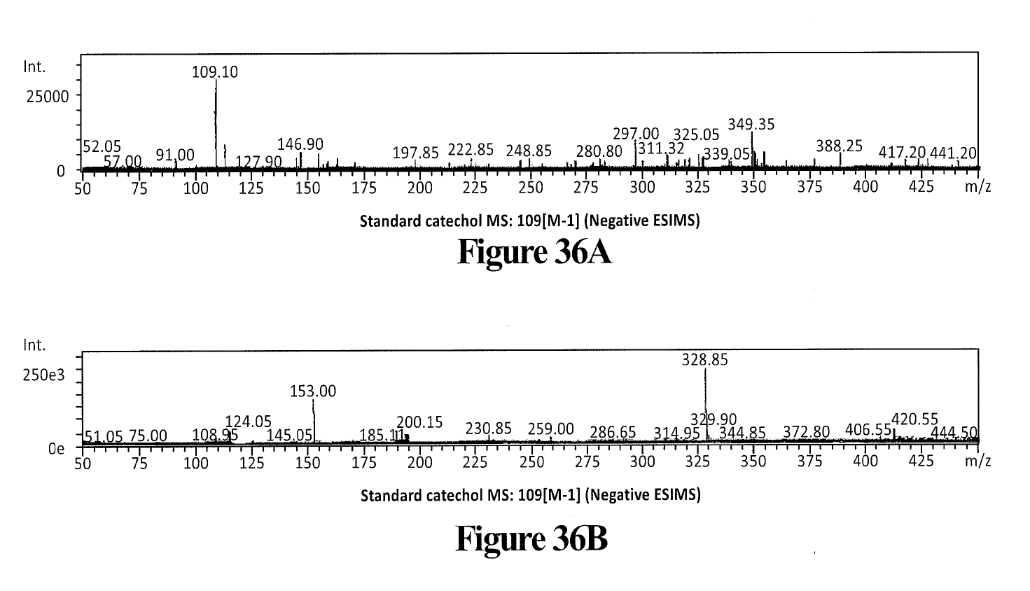

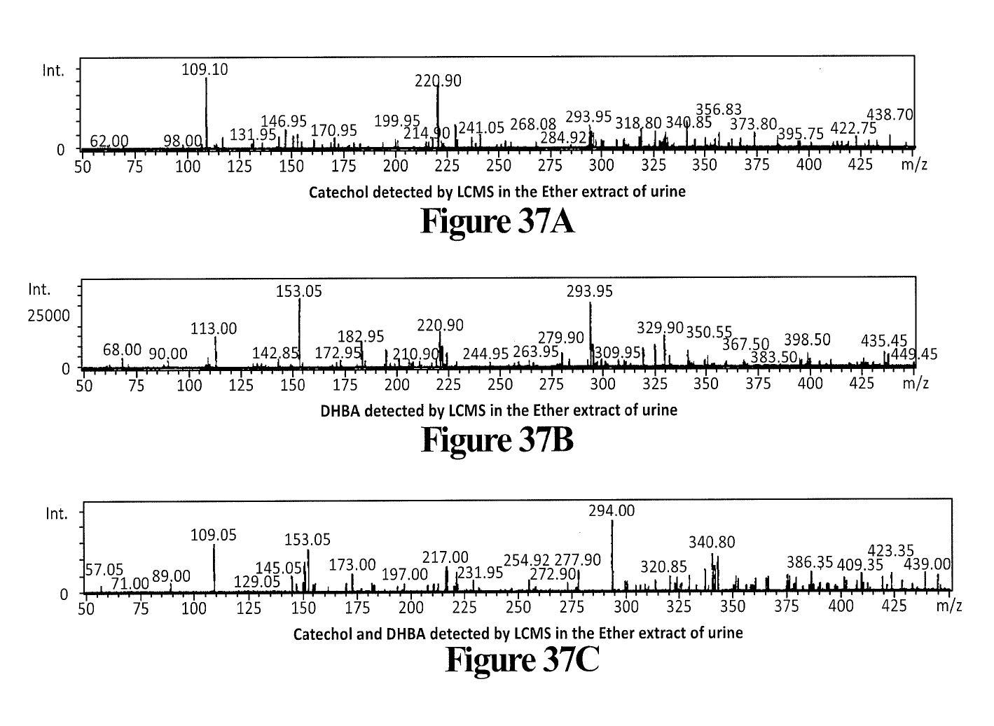

Catechol can be an NGAL siderophore. The amount of catechol in urine by our measurements is 0.6 μg/ml or about 6 04. Catechol itself is the 24 minute peak in the chromatograms depicted in FIGS. 27A-E.

Chemicals and materials. All HMDB (Human Metabolome Database, http://www.hmdb.ca/) Compounds tested (Table 1) were obtained commercially. Methanol, Ethyl acetate, water were of HPLC grade from Fisher, USA. Acetic acid glacial, Tris base, NaCl were from Fisher, USA. DMSO, Ferric Chloride as FeCl3.6H2O, I2, Catchechol-14C (50 mCi/mmol) were from Sigma, USA. Iron-55 as 55FeCl3 PerkinElmer Life and Analytical Sciences, USA, Enterochelin and Ferric Enterochelin were from Biophore Research Products, EMC microcollections GmbH, Germany. Thin layer chromatography plates and chromatography paper were from Whatman. Ngal was isolated by a method published on Mol Cell. 2002 November; 10(5):1045-56. Urine was pooled from volunteered healthy Medical school students, faculty, and patients of Columbia University Medical center.

Use of mice was approved by the Institutional Animal Care and Use Committee of Columbia University.

UV was detected on a Ultrospec 3300 pro UV/visible Spectrophotometer from Amersham Biosciences. ESI Mass was carried on a Shimadzu 2010 LCMS spectrophotometer. 1H-NMR spectra were recorded on a Varian-300 instrument in CD3OD. Radioactive counts were read on a TRI-CARB 2100TR Liquid Scintillation Analyzer from PAKARD.

Iron-binding cofactor: Cofactor-dependent iron binding to Ngal was measured in 150 mM NaCl/20 mM Tris (pH 7.4) buffer (100 μl) with apoNgal (10 μM), 55Fe (1 μM), and HMDB compounds (10 μM). After 60 minutes at room temperature, the mixture was then washed 3 times on a 10-kDa membrane (Amicon YM-10; Millipore Corp.). Ngal loaded with iron-free enterochelin (rather than apo-Ngal) served as a positive control for iron capture. Ferric citrate (1 mM) or iron-loaded enterochelin (siderophore:Fe, 50 10 μM) was used as a competitor of 55Fe binding. (The Journal of Clinical Investigation Volume 115 Number 3 March 2005).

Spectra of various form of Ngal, all at approximately 170 μM, are displayed. (Molecular Cell, Volume 10, Issue 5, Pages 1033-1043)

Paper chromatography: The Fe-Siderophore Binding Assay: The concept underlying the Ngal-siderophore-Fe binding assay is that the urinary molecule provides an iron binding siderophore, and that the Ngal protein provides the carrier which retains the siderophore:Fe on the 10 KDa filter. However, Fe retention does not directly demonstrate that the urine factor itself binds iron, which is a prerequisite for designation as a siderophore. To directly test this idea, we will mix 55Fe with HMDB compounds and then separate bound from free 55Fe by using paper chromatography. Experiments showed that 55Fe is bound by molecule(s) in the urine and rendered mobile on paper chromatography, whereas it is retained at the origin in the absence of the urinary fraction. This assay is highly reproducible and it confirms the existence of a urinary siderophore. The assay also provides an independent means to follow the purification of the urinary siderophore. Using this assay, we can measure the specific activity of the fractions to identify the peak activity, as well as to compare the urinary fraction with enterochelin to establish relative units of activity.

Column chromatography for isolation of Ngal and its siderophore complex: pH7.4 Tris solution as eluent. First equilibrate the column with 5 column volume Tris eluent (2.5 mL*5), then discard the first 2.5 ml eluent, collected the next 3.5 mL eluent, and take 5% of this 3.5 mL to measure on the machine. Mol Cell. 2002 Nov;10(5):1045-56

Sample preparation for quantification of catechol: Urine samples were pooled, filtered through a Whatman paper, and store at −80° C., then filtered the dissolved urine on whatman paper, a 0.22 μm bottle top filter, and 10K membrane filter successively.

Samples preparation for quantification of radioactive catechol in mouse metabolites. Urine samples were pooled, filtered through a Whatman paper, and store at −80° C., then filtered the dissolved urine on whatman paper, a 0.22 μm bottle top filter, and 10K membrane filter successively. Concentrated hydrochloric acid (400 μL) was added to an aliquot (2 mL) of pooled human urine and was then heated in a boiling water bath for 90 min. After the mixture was cooled to room temperature, standard catechol (10 uL 1 mg/mL) and water (5 mL) were added, the solution was extracted with EtOAc (20 mL). The organic layer was freed from solvent, and the residue was taken up in methanol (500 μL) and then analyzed by HPLC.

Quantification of Dihydroxybenzene in urine: extract the prepared urine with Ethyl Acetate for three times. The organic layer was freed from solvent, and the residue was taken up in methanol, and then analyzed by HPLC together with ESIMS (negative mode).

HPLC Analysis: normal analytical work was carried on Waters model 996 with 515 pump. After sample injection (20 μL), chromatographic separation was carried out on a 2.1 mm×150 mm, i.d., 3.5 μm, C-18 Column (Waters, SunFire, made in Ireland) with gradient elution at a flow rate of 0.15 mL/min. Eluent A was 0.5% acetic acid in methanol, and eluent B was 0.5% acetic acid in water. For chromatography, eluent A was increased linearly to 8% within 5 min, then increased linearly to 15% within 45 min, then to 100% within additional 5 min, followed by isocratic elution with 100% for 10 min.

HPLC Analysis of radioactive metabolites of Triptophan-3H: Studies were performed using mouse organs and was carried on Waters 600 HPLC with U6K injector and Perkin-Elmer LC-95 UV/Visible spectrophotometer detector. After sample injection (10 μL), chromatographic separation was carried out on a 4.6 mm×150 mm, i.d., 3.5 μm, C-18 Column (Waters, SunFire, made in Ireland) at a flow rate of 0.5 mL/min. Eluent A was 0.5% acetic acid in methanol, and eluent B was 0.5% acetic acid in water. For chromatography, eluent A was held at 10% within 45 min., then to 100% within additional 5 min, followed by isocratic elution with 100% for 10 min. Collected the peak between 25 min and 29 min and count them.

Calibration: Solutions of the standard catechol were prepared in 7 mass ratios from 0.01 to 0.5 ug, and analysis was performed. Calibration curves were prepared by plotting peak area ratios of analyte to internal standard against concentration ratios of each analyte to the internal standard using linear regression.

Synthesis of catechol cyclic sulfate: Following the method described in the literature (J. Org. Chem 1980; 45: 5371-5373) with slight modification. A 1.1 g (0.01 mol) sample of catechol was dissolved in 1.6 g of pyridine and the mixture stirred vigorously with an stirrer under dry argon in a 100 ml round bottom flask. A 10 mL portion of hexane was then added, after which the reaction mixture was cooled to −5 ° C. in an ice salt bath. A solution of 1.36 g (0.82 mL) of sulfuryl chloride in 2 mL of htexane was then added dropwise over 2 h while the temperature was carefully maintained between −5 and 0 ° C. Stirring at 0 ° C. was continued overnight, after which the reaction mixture was allowed to warm to ambient temperature over 6 h. The upper layer of the two-layer reaction mixture was decanted, after which the lower layer was washed (2×2 mL) with ethyl acetate. The combined washes and upper layer were then washed with 5% CU(OAC)2.H2O with TLC (hexane-ethylacetate, 3:1) indicated the absence of catechol (Rf=0.14). The solution was then dried over magnesium sulfate and concentrated, yielding 1.8 g of an amber liquid. Recrystallization from hexane yielded 0.8 g of long colorless needles, mp 35-36° C. (lit.s mp 34-35° C.). 1H NMR (300 MHz, CD3OD, δ ppm): 7.31 (2H, m), 7.24(2H, m), (catechol: 6.24 and 6.41 ppm, respectively).

Synthesis of catechol sulfate: Following the method described in the literature (JACS 1968; 90(17): 4626-4628) with slight modification. 50 mg of catechol cyclic sulfate in a 20 mL, round bottom flask was added 1.2 mL acetonitrile and 1 mL 0.1 N NaOH solution. The flask was stoppered, shaken vigorously, and allowed to stand at room temperature for 3 h, then extracted by chloroform three times (3 mL, 1 mL, 1 mL respectively). The residue aqueous fraction was dried and washed by absolute ethanol. The total weight of the residue after extraction was 35 mg. 1H NMR (300 MHz, CD3OD, δ ppm): 7.27 (1H, dd, J=8.1, 1.8 Hz), 7.04 (1H, td, J=7.8, 1.5 Hz), 6.79 (1H, dd, J=8.1, 1.5 Hz), 6.62 (1H, td, J=7.8, 1.5 Hz).

Results

Screening Compounds from Human Urine

Human Metabolome Database reported iron binding activity in a series of urinary compounds. Selected 57 compounds were tested, 15 of which showed iron binding activity by paper chromatography and of those 6 showed protein iron binding activity by filter retention assay (Ngal-Siderophore-Fe Binding Assay).

Ngal-Siderophore-Fe Binding Assay result is shown in FIG. 1: Enterochelin; Myricetin (GB1-61-1); Ellagic acid from chestnut bark (GB1-61-3); 2, 3-Dihydroxybenzoic acid (GB1-49-1); Rosmarinic acid (GB1-49-4); Catechol (GB1-56-3); (−)-Epigallocatechin gallate from green tea, >80% (GB1-59-4), among which Catechol (GB1-56-3) is the most active compound. All six active compounds can be inhibited by 50 fold Enterochelin:Fe (FIG. 1).

Iron chelating activity by paper chromatography: compounds 2,3-Dihydroxybenzoic acid (GB1-49-1), Rosmarinic acid (GB1-49-4), Sodium Citrate (GB1-54-4), Catechol (GB1-56-3), 3-Hydroxy-DL-kynurenine (GB1-58-4), β-Nicotinamide adenine (GB1-58-5), DL-Isocitric acid (GB1-59-1), Chlorogenic acid (GB1-59-2), Epigallocatechin gallate from green tea, >80% (GB1-59-4), Flavin adenine dinucleotide disodium salt hydrate (GB1-59-5, FAD), β-Nicotinamide adenine dinucleotide phosphate sodium salt (GB1-59-6, NADP), 3,4-dihydroxy-DL-phenylalanine (GB1-59-10), 3,4-dihydroxy-L-phenylalanine (GB1-61-2), Ellagic acid from chestnut Bark (GB1-61-3), 3, 4-Dihydroxybenzoic acid (GB1-61-6) were found to be active on chelating iron by paper chromatography.

### Example 7

### Small Scale Fractionation of Human Urine and Mice Urine

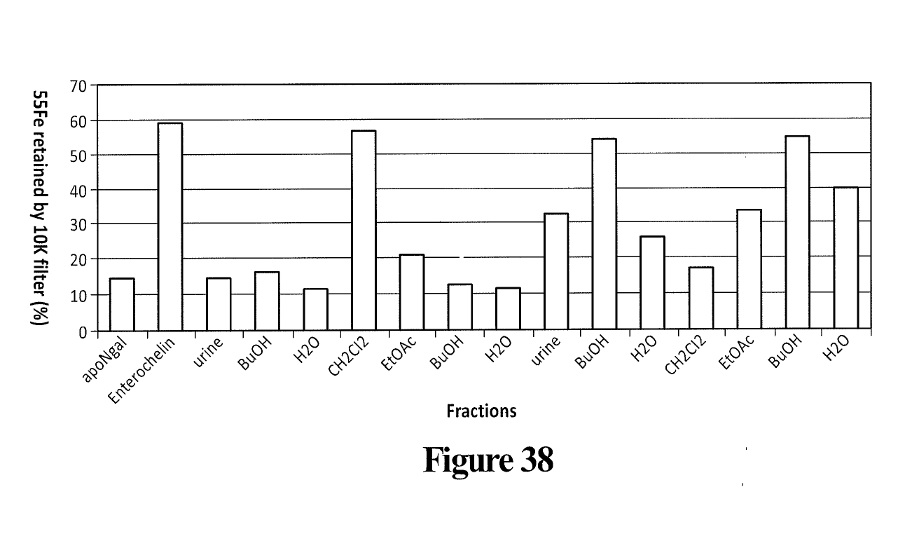

Sixty-six small fractions were prepared and tested with the Ngal siderophore iron binding assay. The assay showed that the dicholoromethane fraction (GB 1-51-4) is the most active fraction fo human urine extract, while n-butanol and water fractions (GB1-52-5, GB1-52-6, GB1-52-7) are the active fractions of mice urine extracts. See FIG. 38 for protein iron binding activity.

### Example 8

### Catechol, a Urinary Ngal Binding Siderophore

NGAL (siderocalin), is a carrier protein that is expressed by neutrophils and by epithelia stimulated by iron, hypoxia and growth factors. Functionally, NGAL binds enterochelin:Fe, a bacterial siderophore, but since NGAL is expressed in sterile forms of renal failure, NGAL may bind additional organic chemicals. To identify these molecules, a candidate molecule approach was used, as well as purification from 400 liters of human urine endogenous NGAL siderophores. It has been established that catechol solubilized iron binds NGAL with a spectral shift similar to enterochelin and retains iron in an NGAL complex, even after days of washing. Crystallographic evidence further demonstrates occupancy of the NGAL calyx. This is the first report of Ngal:mammalian siderophore complex as the principal urinary non-transferring bound iron pool carrier. (Yang J, et al. (2002) Mol. Cell 10: 1045-1056; Mori K, et al. (2005) J. Clin. Invest. 115: 610-621)

### Example 9

### Rapid Diagnostic Test for Urinary Ngal

The power of NGAL in clinical AKI is the result of its rapid expression. Time lost to obtain results from a formal laboratory would defeat its purpose. In this aim we will develop a rapid, inexpensive (<$1), reliable, single step semi-quantitative assay for urinary NGAL which can be unambiguously interpreted and stable in wide varieties of climates, in any country. The objective is to detect an increase in NGAL from the background concentrations of <20 ng/ml (up to 2 μg/ml) against a background of other urinary proteins (normal range up to 80 μg/ml, and pathological range up to 12 gr/l). An immunochromatographic technique, known as the lateral flow rapid test (Zhang C, Zhang Y, Wang S. Development of multianalyte flow-through and lateral-flow assays using gold particles and horseradish peroxidase as tracers for the rapid determination of carbaryl and endosulfan in agricultural products. J Agric Food Chem. 2006 Apr. 5; 54(7):2502-7), can be adapted and formatted as a free standing dipstick. For the affinity capture of NGAL, a siderophore analog may be used, and commercially available (Schleicher&Schuell) kits (which include pre-made lateral flow cassettes) can be used for the development of such an assay.

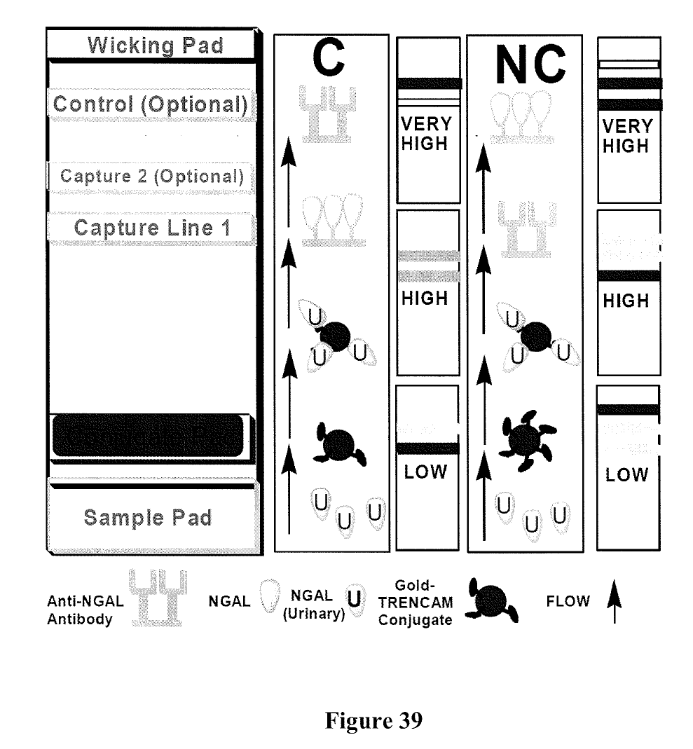

A competitive lateral flow assay can be analyzed based on the following principles (FIG. 39, Left panel and C): On a nitrocellulose strip we will immobilize NGAL (Capture Line 1, with further capture lines optional). A glass fiber pad containing dried gold nanoparticles conjugated with a siderophore analog (e.g. TRENCAM on BSA) can be attached to the strip. The urine sample with NGAL will migrate by capillary diffusion through the conjugate pad, rehydrating the gold-TRENCAM conjugate, and binding to it. Excess gold-TRENCAM conjugate will move onto membrane strip, where it will be captured by immobilized NGAL, producing a signal in the form of a sharp red line. To achieve better quantification, excess gold-TRENCAM conjugates can captured by subsequent strips of NGAL (e.g., capture line 2), thus producing a ladder of lines on the strip. A control capture line (e.g. with an anti-NGAL antibodies) to capture gold-TRENCAM-NGAL complexes conjugates can be added, to further facilitate quantification. In this format, high concentration of NGAL in urine results in strongly red colored control line, but little color in capture line 1.

At least two alternative formats can also be compared: (1) direct (noncompetitive format) using polyclonal antibodies against NGAL in capture lines (FIG. 39, NC); and (2) competitive format with gold-nanoconjugates with NGAL competing with urine NGAL for capture by a TRENCAM-BSA conjugate in a capture layer.

For each assay one can optimize amounts and structures of capture agents and gold-nanoconjugates to achieve optimal sensitivity and minimize false positive results. Initial tests of all reagents can be performed using surface plasmon resonance instrument. Sensitivity of these tests is limited by the ability of the user to visually detect the gold signal on a white membrane, but this can be overcome by using silver enhancement.

For the siderophore conjugates enterobactin is not recommended, because of the instability of the ester bonds. NGAL binds stable analogs TRENCAM and MECAM (Holmes MA, Paulsene W, Jide X, Ratledge C, Strong R K. Siderocalin (Lcn 2) also binds carboxymycobactins, potentially defending against mycobacterial infections through iron sequestration. Structure. 2005 13(1):29-41), and it will likely bind the enterobactin analog derived from myo-inositol (Tse, B and Kishi Y. Chiral Analogs of Enterobactin with hydrophilic or lipophilic properties. J. Am. Chem. Soc. 115: 7892-7893. 1993). The analogs of these three compounds, which are suitable for the conjugation to large proteins and then to gold particles, are readily accessible through chemical synthesis.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways within the scope and spirit of the invention.

1. A pharmaceutical composition comprising a lipocalin and a compound of Formula I 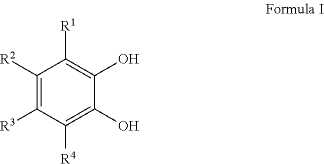
or a pharmaceutically acceptable salt or hydrate thereof, wherein:
R¹ is H, halogen, OR⁵, N(R⁵)₂, NO₂, N₃, CN, CO₂R⁵, —C(═O)N(R⁵)₂, S(R⁵), SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl, or R⁶;
R² is H, halogen, OR⁵, N(R⁵)₂, NO₂, N₃, CN, CO₂R⁵, —C(═O)N(R⁵)₂, S(R⁵), SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl, a carbonyl forming an ester with a hydroxyl at the 3-position of a catechol, or R⁶;
R³ is H, halogen, OR⁵, N(R⁵)₂, NO₂, N₃, CN, CO₂R⁵, —C(═O)N(R⁵)₂, S(R⁵), SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl, catechol-4-yl, 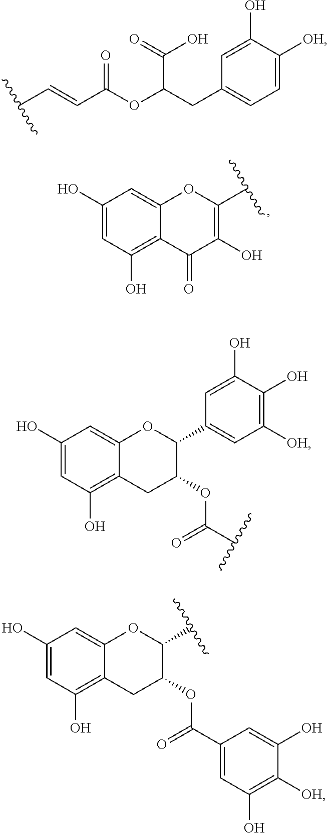
or R⁶, wherein the C₁₋₆ alkyl is optionally substituted with 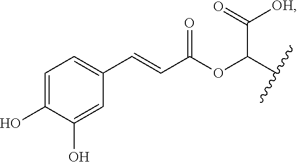
and the catechol-4-yl is optionally substituted with a 5-CO₂R⁵, a 3-OR⁵, or both, or two compounds of formula I are bonded together at the R³ positions, or two compounds of formula I are bonded together at the R³ positions where R² is —CO₂R⁵ and R⁴ is —OR⁵, or two compounds of formula I are bonded together at the R³ positions where R² is —CO₂R⁵ and R⁴ is —OR⁵ and the R² acyl groups form esters with the R⁴ hydroxyl group of the other compound;
R⁴ is H, halogen, OR⁵, N(R⁵)₂, NO₂, N₃, CN, CO₂R⁵, —C(═O)N(R⁵)₂, S(R⁵), SO₃(R⁵), SO₂N(R⁵)₂, C₁₋₆-alkyl, C₁₋₆-alkyl-OR⁵, C₁₋₆-alkyl-N(R⁵)₂, C₁₋₆-alkyl-CO₂R⁵, C₃₋₁₀ aryl, —O—C₃₋₁₀ aryl, —NR⁵—C₃₋₁₀ aryl, —S—C₃₋₁₀ aryl, a hydroxyl forming an ester with a carbonyl at the 5-position of a catechol, or R⁶;
each R⁵ is independently H or C₁₋₆ alkyl;
R⁶ is 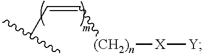
X is —NR⁵—, —O—, —C(═O)O—, or —C(O)NR⁵—;
Y is H, —C(═O)R⁵, C₁₋₆-alkyl, C₃₋₁₀ aryl, C₃₋₁₀ cycloalkyl, or C₁₋₆ heterocyclyl;
m is an integer ranging from 0 to 2; and
n is an integer ranging from 0 to 4, and wherein the compound is not dihydroxybenzoic acid or N-dihydroxybenzoyl-serine.

2. The pharmaceutical composition of claim 1, wherein the compound of Formula I has the Formula Ia 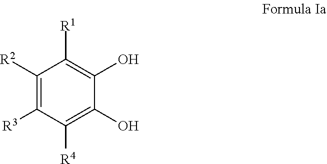
Wherein R¹ is H or OR⁵;
R² is H or carbonyl forming an ester with a hydroxyl at the 3-position of a catechol;
R³ is H, C₁₋₆ alkyl, catechol-4-yl, 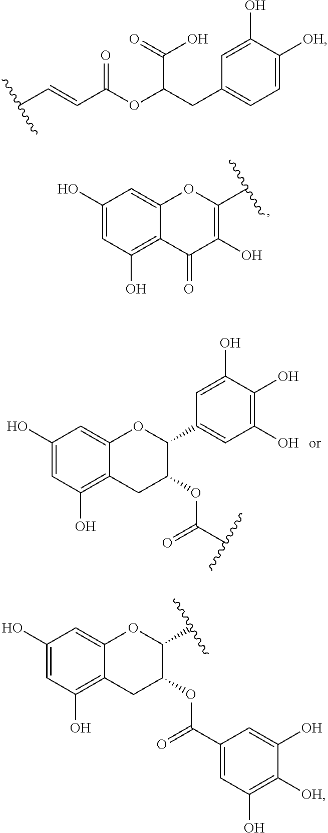
wherein the C₁₋₆ alkyl is optionally substituted with 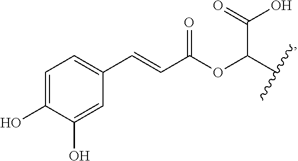
and the catechol-4-yl is optionally substituted with a 5-CO₂R⁵, a 3-OR⁵, or both;
R⁴ is H, C₁₋₆ alkyl, OR⁵, CO₂R⁵, or hydroxyl forming an ester with a carbonyl at the 5-position of a catechol; and
each R⁵ is independently H or C₁₋₆ alkyl,
and wherein the compound is not dihydroxybenzoic acid or N-dihydroxybenzoyl-serine.

3. The pharmaceutical composition of claim 1, wherein the compound of Formula I is selected from the group consisting of: catechol, 3-methylcatechol, 4-methylcatechol, rosmarinic acid, myricetin, epigallocatechin gallate, pyrogallol, and ellagic acid.

4. The pharmaceutical composition of claim 1, wherein the compound of Formula I is bound to the lipocalin.

5. The pharmaceutical composition of claim 1, wherein the lipocalin is NGAL.

6. The pharmaceutical composition of claim 5, wherein the NGAL has the sequence of SEQ ID NO. 1 or SEQ ID NO. 2, or is a variant, homolog, derivative, fragment or mutant thereof that has the ability to bind to the compound of Formula I.

7. The pharmaceutical composition of claim 5, wherein the NGAL has the sequence of SEQ ID NO. 1 or SEQ ID NO. 2.

8. The pharmaceutical composition of claim 1, wherein the composition further comprises iron.

9. The pharmaceutical composition of claim 8, wherein the iron is bound to the composition of Formula I.

10. A method for treating iron deficiency, the method comprising administering to a subject in need thereof a therapeutically effective amount of a pharmaceutical composition of any of claims 1-8.

11. The method of claim 10, wherein the iron deficiency is associated with anemia, chronic hemodialysis, peritoneal dialysis, End Stage Renal Disease (ESRD), chronic kidney disease (CKD), cancer, HIV/AIDS, hepatitis, autoimmune diseases, cardiovascular disease, loss of blood, chronic bleeding, pregnancy, use of drugs that interfere with iron absorption, nutritional iron deficiency, iron malabsorption, Crohn's disease, celiac sprue, fever, hemosiderinuria, pulmonary siderosis, inflammation, or any combination thereof.

12. A method for treating iron overload, the method comprising administering to a subject in need thereof a therapeutically effective amount of a pharmaceutical composition of any of claims 1-8.

13. The method of claim 12, wherein the iron overload is associated with hemochromatosis type 1 (classical hemochromatosis), hemochromatosis type 2A or 2B (juvenile hemochromatosis), hemochromatosis type 3, hemochromatosis type 4 (African iron overload), neonatal hemochromatosis, aceruloplasminemia, congenital atransferrinemia, dietary iron overload, transfusional iron overload, hemodialysis, chronic liver disease, hepatitis C, cirrhosis, non-alcoholic steatohepatitis, porphyria cutanea tarda, post-portacaval shunting, dysmetabolic overload syndrome, or iron tablet overdose, or any combination thereof.

14. The method of claim 12, wherein the iron overload is in the kidneys and wherein the iron overload is associated with oxidative injury in the proximal tubules, acute tubular necrosis (ATN), renal failure, ischemia of the kidneys, or exposure to nephrotoxic agents.

15. A method for treating iron toxicity in a subject in need thereof, the method comprising removing a blood sample from the subject, adding to the blood sample a pharmaceutical composition of any one of claims 4-7, wherein iron in the blood sample binds to and is chelated by the composition, and returning the blood sample to the subject.

16. The method of claim 15, wherein the pharmaceutical composition is removed from the blood sample is removed from the blood sample before it returned to the subject.

17. A method for detecting the presence of a lipocalin in a sample, the method comprising: (a) contacting a siderophore with iron, thereby forming a complex between the siderophore and the iron; (b) contacting the sample with the complex of step (a); and (c) determining whether, after contacting with the sample, the complex contains a lipocalin.

18. The method of claim 17, wherein the siderophore is a compound of Formula I, I(a), I(b), II, or III.

19. The method of claim 17, wherein the lipocalin is NGAL.
